(19) United States
(12) Patent Application Publication
(10) Pub. No.: US 2025/0228858 A1
(43) Pub. Date: Jul. 17, 2025

(54) ANTI-CANCER COMBINATION THERAPY

(71) Applicant: Boehringer Ingelheim International GmbH, Ingelheim am Rhein (DE)

(72) Inventors: Martin AUGSTEN, Vienna (AT); Anke BAUM, Moedling (AT); Krzysztof CHYLINSKI, Vienna (AT); Irene WAIZENEGGER, Vienna (AT)

(21) Appl. No.: 18/985,218

(22) Filed: Dec. 18, 2024

(30) Foreign Application Priority Data
Dec. 20, 2023 (EP) 23218705.4
Jul. 12, 2024 (EP) 24188282.8

(51) Int. Cl. A61K 31/519; A61K 31/337; A61K 31/357; A61K 31/505; A61K 31/513; A61K 39/00; A61K 47/68; A61P 35/00; C07K 16/32

(52) U.S. Cl. CPC A61K 31/519; A61K 31/337; A61K 31/357; A61K 31/505; A61K 31/513; A61K 47/6803; A61K 47/6855; A61P 35/00; C07K 16/32; A61K 2039/505

(57) ABSTRACT

The present invention refers to the combination of the HER2 tyrosine kinase inhibitor zongertinib with an anti-HER2 antibody and/or an anti-HER2 antibody-drug conjugate. Such combination may further comprise an additional anti-cancer medicament. The combination of the invention is useful for the treatment and/or prevention of oncological and/or hyperproliferative diseases, such as cancer. Accordingly, compounds for use, methods of prevention and/or treatment, uses, pharmaceutical compositions and kits related to the combination are herewith provided.

### FIELD OF THE INVENTION

The present invention refers to the combination of the HER2 tyrosine kinase inhibitor zongertinib, or a pharmaceutically acceptable salt thereof, with an anti-HER2 antibody and/or an anti-HER2 antibody-drug conjugate. Such combination may further comprise an additional anti-cancer medicament. The combination of the invention is useful for the treatment and/or prevention of oncological and/or hyperproliferative diseases, in particular cancer.

### BACKGROUND OF THE INVENTION

Human epidermal growth factor receptor 2 (HER2; encoded by ERBB2) belongs to the family of ERBB transmembrane receptor tyrosine kinases.

Aberrant activation of HER2 through different mechanisms plays a crucial role in the development and progression of a variety of cancers. For example, more than 20% of human breast cancers overexpresses HER2 resulting from ERBB2 amplification and manifests a historically poor prognosis (Swain, S. M., Shastry, M. & Hamilton, E. Nat. Rev. Drug Discov. 22, 101-126; 2023). Also in 12 to 20% of metastatic gastric, gastroesophageal junction or esophageal adenocarcinoma cases, HER2 is overexpressed (Jorgensen J T, Hersom M. J Cancer; 3, p. 137-144; 2012; Wu H, et al. Tumori; 103 (3), p. 249-254; 2017).

In addition to overexpression, HER2 can be aberrantly activated by oncogenic mutations in multiple solid cancers. For instance, HER2 mutations occur in 2-4% of non-small cell lung cancers (NSCLC) and have emerged as important oncogenic drivers (Connell, C. M. & Doherty, G. J. Esmo Open 2, e000279 (2017); Yan, M., et al. Cancer Treat Rev; 40, 770-780 (2014); Stephens, P. et al. Nature 431, 525-526 (2004)). This subset of NSCLC, especially those carrying the most common 12 base pair insertion in ERBB2 exon 20 resulting in the duplication of the amino acids YVMA in HER2 (HER2^{YVMA}), is associated with aggressive disease progression and poor clinical outcomes in response to chemotherapy and immunotherapy.

ERBB signaling can also be aberrantly activated through alterations in the ligands to the receptors of the ERBB family. In this context, fusions of the neuregulin-1 gene (NRG1), although very rare, have been clinically documented (Nagasaka, M. & Ou, S. H. I. Trends Cancer 8, 242-258 (2022)). Current therapeutic options to address cancers with HER2 aberrant activation include monoclonal HER2-directed antibodies (e.g. trastuzumab, pertuzumab and margetuximab) and HER2-directed antibody-drug conjugates (ADCs), such as trastuzumab emtansine (T-DM1, Kadcyla) and trastuzumab deruxtecan (T-DXd, Enhertu). These anti-HER2 antibodies and ADCs target and can bind to an extracellular domain of HER2. Although both ADCs rely on trastuzumab to bind to HER2 expressing cells, they differ in the attached cytotoxic moiety. T-DM1 utilizes emtansine, which is a microtubule inhibitor whereas T-DXd includes deruxtecan, a topoisomerase I inhibitor.

Small molecule tyrosine kinase inhibitors (TKI) are also known. They target and can bind to an intracellular domain of HER2, such as the tyrosine kinase domain. For example, tucatinib is a HER2 inhibitor approved for the treatment of HER2 positive breast cancer in combination with trastuzumab and capecitabine.

Zongertinib is a potent and selective covalent tyrosine kinase inhibitor of wild type and mutant HER2 that spares wild type epithelial growth factor receptor (EGFR; also referred to as HER1 and encoded by EGFR), another member of the ERBB family. Zongertinib is described in WO 2021/213800 and in Wilding B., et al.; Cancer Discov; 2024; XX; 1-20. It represents a promising treatment option for HER2-dependent cancers.

Therapeutic resistance and disease relapse are known in patients treated with anti-HER2 monoclonal antibodies or ADCs. The combination of a small molecule tyrosine kinase inhibitor with a HER2-targeting antibody or ADC may help overcome resistance to antibody-mediated inhibition through utilization of an alternative mechanism of receptor inhibition. In addition, pan-ERBB inhibitors were shown to induce increased ADC internalization through receptor ubiquitination (Li B T, et al. Cancer Discov; 2020; 10 (5); 674-687; Olson D, et al. Cancer Res Commun. 2023; 3 (9): 1927-1939). It is however not clear if a covalent HER2 selective, EGFR WT sparing compound would serve as a good combination partner for HER2-directed ADCs.

WO 2018/201016 describes a method for treating a HER2-positive cancer comprising administering a combination therapy including tucatinib and an anti-HER2 antibody, such as trastuzumab, pertuzumab, T-DM1 or margetuximab.

WO 2021/097220 refers to a method for treating a HER2-positive breast cancer by administration of a combination of tucatinib and an anti-HER2 antibody-drug conjugate, such as T-DM1 or T-DXd.

WO 2022/067347 concerns a method for treating a solid tumor with one or more HER2 alterations using a combination of tucatinib and at least one anti-HER2 antibody, such as trastuzumab. Despite all these approaches, there is still a need for improved treatment options for cancer patients. It is therefore an object of the present invention to provide anti-cancer therapies with therapeutic efficacy and safety.

### SUMMARY OF THE INVENTION

In the present invention, it was found that zongertinib potently inhibits the proliferation of HER2-dependent cells that have acquired resistance to the HER2-directed ADC T-DXd (Example 5). In addition, the combination of zongertinib with anti-HER2 antibody trastuzumab or pertuzumab or with anti-HER2 ADC T-DXd or T-DM1 inhibited tumor growth and induced tumor shrinkage in xenografts derived from HER2-overexpressing gastric, esophageal, breast, lung or ovarian cancer cell lines as well as from a HER2 mutant NSCLC cell line, while being well tolerated (Examples 1-3, 7, 8, 10-15). Remarkably, the above-mentioned combinations led to deeper tumor regressions than respective single agent therapies, achieving even complete regressions (Examples 1-3, 7, 14-15). Combined anti-proliferative activity was also observed for zongertinib plus T-DXd, trastuzumab or pertuzumab in different HER2 amplified breast cancer cell lines (Example 4). Triple combinations of zongertinib+the anti-HER2 antibody trastuzumab or the anti-HER2 ADC T-DXd+pertuzumab performed better than corresponding dual combinations in xenograft models of HER2-overexpressing breast or lung cancer (Examples 10, 13, 14). Combining trastuzumab and capecitabine with zongertinib was also more efficacious than corresponding dual combinations in a HER2-overexpressing breast cancer xenograft model (Example 9). Advantageously, zongertinib enhances the activity of antibody-drug conjugates, without causing obvious toxicities. Therefore, combinations of zongertinib with HER2-directed antibodies or ADCs may expand the population of patients who would benefit from HER2 targeting antibodies or ADCs. Specifically, it has been shown herein that there is a synergistic effect, defined as a greater than additive benefit, provided by co-administration of zongertinib or a pharmaceutically acceptable salt thereof and a combination partner as defined herein in the treatment of cancer. It is believed that, by acting simultaneously, superior inhibition of cancer can be achieved leading to improved cancer treatment outcomes, at least for cancers normally susceptible to treatment by anti-HER2 therapies.

The present invention thus provides a combination of zongertinib or a pharmaceutically acceptable salt thereof with an anti-HER2 antibody and/or an anti-HER2 antibody-drug conjugate. In particular, the present invention provides a combination of zongertinib or a pharmaceutically acceptable salt thereof with an anti-HER2 antibody or an anti-HER2 antibody-drug conjugate. An additional anti-cancer medicament could be added to said combination(s). In this context, the anti-cancer medicament is additional to zongertinib or the pharmaceutically acceptable salt thereof and to the anti-HER2 antibody and/or the anti-HER2 antibody-drug conjugate. The present invention thus also provides a combination of zongertinib or a pharmaceutically acceptable salt thereof with an anti-HER2 antibody and/or an anti-HER2 antibody-drug conjugate and with an anti-cancer medicament. In an embodiment, the combination is administered in an amount to result in a synergistic therapeutic effect. The combination is particularly useful in the treatment and/or prevention of cancer, especially wherein said cancer is HER2 mutant, HER2 overexpressed and/or HER2 amplified.

According to a first aspect is provided zongertinib or a pharmaceutically acceptable salt thereof for use in the treatment and/or prevention of cancer, wherein zongertinib or the pharmaceutically acceptable salt thereof is administered in combination with a combination partner, wherein said combination partner is selected from the group consisting of an anti-HER2 antibody, an anti-HER2 antibody-drug conjugate and a combination thereof. In an embodiment of this aspect, zongertinib or the pharmaceutically acceptable salt thereof and the combination partner are administered in combination with an anti-cancer medicament. In other words, said first aspect provides zongertinib or a pharmaceutically acceptable salt thereof for use in the treatment and/or prevention of cancer, wherein zongertinib or the pharmaceutically acceptable salt thereof is administered in combination with a combination partner as defined herein and optionally in further combination with an anti-cancer medicament.

Another aspect relates to an anti-HER2 antibody, an anti-HER2 antibody-drug conjugate or a combination thereof for use in the treatment and/or prevention of cancer, wherein the anti-HER2 antibody, anti-HER2 antibody-drug conjugate or combination thereof is administered in combination with zongertinib or a pharmaceutically acceptable salt thereof. In an embodiment of this aspect, the anti-HER2 antibody, anti-HER2 antibody-drug conjugate or combination thereof and zongertinib or the pharmaceutically acceptable salt thereof are administered in combination with an anti-cancer medicament.

Another aspect relates to a method of treating and/or preventing cancer, the method comprising administering to a patient in need thereof: zongertinib or a pharmaceutically acceptable salt thereof, and a combination partner, wherein said combination partner is selected from the group consisting of an anti-HER2 antibody, an anti-HER2 antibody-drug conjugate and a combination thereof. In an embodiment, such method comprises administering to a patient in need thereof: a therapeutically effective amount of zongertinib or a pharmaceutically acceptable salt thereof, and a therapeutically effective amount (especially a synergistically therapeutically effective amount) of the combination partner as defined herein. In addition or in alternative, in an embodiment, the method further comprises administering to a patient in need thereof an anti-cancer medicament, in particular a therapeutically effective amount of an anti-cancer medicament.

Another aspect relates to a use of zongertinib or a pharmaceutically acceptable salt thereof for the manufacture of a medicament for the treatment and/or prevention of cancer, wherein zongertinib or the pharmaceutically acceptable salt thereof is administered in combination with a combination partner, wherein said combination partner is selected from the group consisting of an anti-HER2 antibody, an anti-HER2 antibody-drug conjugate and a combination thereof. In an embodiment of this aspect, zongertinib or the pharmaceutically acceptable salt thereof and the combination partner are administered in combination with an anti-cancer medicament.

Another aspect relates to a use of an anti-HER2 antibody, an anti-HER2 antibody-drug conjugate or a combination thereof for the manufacture of a medicament for the treatment and/or prevention of cancer, wherein the anti-HER2 antibody, anti-HER2 antibody-drug conjugate or combination thereof is administered in combination with zongertinib or a pharmaceutically acceptable salt thereof. In an embodiment of this aspect, the anti-HER2 antibody, anti-HER2 antibody-drug conjugate or combination thereof and zongertinib or the pharmaceutically acceptable salt thereof are administered in combination with an anti-cancer medicament.

Another aspect relates to a kit comprising: (i) a first pharmaceutical composition comprising zongertinib or a pharmaceutically acceptable salt thereof and a pharmaceutically acceptable excipient; and (ii) a second pharmaceutical composition comprising a combination partner and a pharmaceutically acceptable excipient; wherein the combination partner is selected from the group consisting of an anti-HER2 antibody, an anti-HER2 antibody-drug conjugate and a combination thereof. In an embodiment, the first pharmaceutical composition comprises a therapeutically effective amount of zongertinib or a pharmaceutically acceptable salt thereof and the second pharmaceutical composition comprises a therapeutically effective amount of the combination partner. In addition or in alternative, in an embodiment, the kit further comprises a third pharmaceutical composition comprising an anti-cancer medicament and a pharmaceutically acceptable excipient, in particular a therapeutically effective amount of an anti-cancer medicament.

Another aspect relates to zongertinib or a pharmaceutically acceptable salt thereof for use in the treatment and/or prevention of cancer, wherein the cancer is resistant to treatment with an anti-HER2 antibody and/or an anti-HER2 antibody-drug conjugate.

Another aspect relates to a method of treating and/or preventing cancer, the method comprising administering to a patient in need thereof zongertinib or a pharmaceutically acceptable salt thereof, wherein the cancer is resistant to treatment with an anti-HER2 antibody and/or with an anti-HER2 antibody-drug conjugate. In an embodiment, such method comprises administering to a patient in need thereof a therapeutically effective amount of zongertinib or a pharmaceutically acceptable salt thereof.

Another aspect relates to a use of zongertinib or a pharmaceutically acceptable salt thereof for the manufacture of a medicament for the treatment and/or prevention of cancer, wherein the cancer is resistant to treatment with an anti-HER2 antibody and/or with an anti-HER2 antibody-drug conjugate. The definitions of zongertinib, combination partner, anti-HER2 antibody, anti-HER2 antibody-drug conjugate, dosing schedule, combination therapy, anti-cancer medicament, additional intervention and cancer provided in the detailed description hereinbelow are applicable to any and all aspects described in the present summary of invention.

In the present invention, any aspect or embodiment referring to a feature (e.g. the identity of the combination partner) can be combined with any one or more aspect(s) or embodiment(s) referring to (an) other feature(s) (e.g. the identity of the cancer) to provide further aspects or embodiments of the invention.

### BRIEF DESCRIPTION OF THE FIGURES

FIG. 1: Tumor growth kinetics in NCI-N87 tumor-bearing mice treated with vehicle, zongertinib, T-DXd or their combination at indicated doses and schedules. Median tumor volumes are plotted over time. Day 1 was the first day, day 22 the last day of the experiment.

FIG. 2: Waterfall plots on day 22 of change of tumor volume relative to day 1 in NCI-N87 tumor-bearing mice treated with vehicle (not shown), zongertinib, T-DXd or their combination at indicated doses and schedules. Each bar refers to one individual tumor. Horizontal lines indicate the RECIST criteria (>20% progressive disease, 20% to −30% stable disease, <−30% partial response, 100% complete response).

FIG. 3: Tumor growth kinetics in NCI-N87 tumor-bearing mice treated with vehicle, zongertinib, T-DM1 or their combination at indicated doses and schedules. Median tumor volumes are plotted over time. Day 1 was the first day, day 21 the last day of treatment.

FIG. 4: Waterfall plots on day 21 of change of tumor volume relative to day 1 in NCI-N87 tumor-bearing mice treated with vehicle (not shown), zongertinib, T-DM1 or their combination at indicated doses and schedules. Each bar refers to one individual tumor. Horizontal lines indicate the RECIST criteria (>20% progressive disease, 20% to −30% stable disease, <−30% partial response, 100% complete response).

FIG. 5: Tumor growth kinetics in PC-9_YVMA-5, EGFR^{KO} tumor-bearing mice treated with vehicle, zongertinib, T-DM1 or their combination at indicated doses and schedules. Median tumor volumes are plotted over time. Day 1 was the first day, day 24 the last day of the experiment.

FIG. 6: Waterfall plots on day 24 of change of tumor volume relative to day 1 in PC-9_YVMA-5 tumor-bearing mice treated with vehicle (not shown), zongertinib, T-DM1 or their combination at indicated doses and schedules. Each bar refers to one individual tumor. Horizontal lines indicate the RECIST criteria (>20% progressive disease, 20% to −30% stable disease, <−30% partial response, 100% complete response).

FIG. 7: Tumor growth kinetics in OE19 tumor-bearing mice treated with vehicle, zongertinib, T-DXd or their combination at indicated doses and schedules. Median tumor volumes are plotted over time. Day 1 was the first day, day 20 the last day of the experiment.

FIG. 8: Waterfall plots on day 20 of change of tumor volume relative to day 1 in OE19 tumor-bearing mice treated with vehicle (not shown), zongertinib, T-DXd or their combination at indicated doses and schedules. Each bar refers to one individual tumor. Horizontal lines indicate the RECIST criteria (>20% progressive disease, 20% to −30% stable disease, <−30% partial response, 100% complete response).

FIG. 9: Cell Growth Inhibition (CGI) values in [%] measured for the combination of zongertinib with T-DXd in the BT-474 cancer cell line. Only one run is herewith illustrated, similar data were obtained in repetitive runs.

FIG. 10: Cell Growth Inhibition (CGI) values in [%] measured for the combination of zongertinib with trastuzumab in the BT-474 cancer cell line. Only one run is herewith illustrated, similar data were obtained in repetitive runs.

FIG. 11: Cell Growth Inhibition (CGI) values in [%] measured for the combination of zongertinib with pertuzumab in the BT-474 cancer cell line. Only one run is herewith illustrated, similar data were obtained in repetitive runs.

FIG. 12: Cell Growth Inhibition (CGI) values in [%] measured for the combination of zongertinib with T-DXd in the SK-BR-3 cancer cell line. Only one run is herewith illustrated, similar data were obtained in repetitive runs.

FIG. 13: Cell Growth Inhibition (CGI) values in [%] measured for the combination of zongertinib with trastuzumab in the SK-BR-3 cancer cell line. Only one run is herewith illustrated, similar data were obtained in repetitive runs.

FIG. 14: Cell Growth Inhibition (CGI) values in [%] measured for the combination of zongertinib with T-DXd in the MDA-MD-453 cancer cell line. Only one run is herewith illustrated, similar data were obtained in repetitive runs.

FIG. 15: Cell Growth Inhibition (CGI) values in [%] measured for the combination of zongertinib with trastuzumab in the MDA-MD-453 cancer cell line. Only one run is herewith illustrated, similar data were obtained in repetitive runs.

FIG. 16: Cell Growth Inhibition (CGI) values in [%] measured for the combination of zongertinib with pertuzumab in the MDA-MD-453 cancer cell line. Only one run is herewith illustrated, similar data were obtained in repetitive runs.

FIG. 17: Schematic representation of the experiment for data in FIG. 18 or 19. T-DXd resistant tumors are generated in vivo, harvested and cultured in vitro, then tested for sensitivities to T-DXd, the T-DXd payload deruxtecan and zongertinib.

FIG. 18: Reduction of phosphorylated HER2 (Y1196) upon zongertinib but not T-DXd treatment in parental and T-DXd resistant NCI-N87 cells.

FIG. 19: Dose response curves (each line represents an independent experiment) of parental and T-DXd resistant NCI-N87 cells to (A) deruxtecan, (B) T-DXd, (C) zongertinib treatment in vitro.

FIG. 20: Tumor growth kinetics in OE19 tumor-bearing mice treated with vehicle, zongertinib, trastuzumab or their combination at indicated doses and schedules. Median tumor volumes are plotted over time. Day 1 was the first day, day 14 the last day of the experiment.

FIG. 21: Waterfall plots on day 14 of change of tumor volume relative to day 1 in OE19 tumor-bearing mice treated with vehicle (not shown), zongertinib, trastuzumab or their combination at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

FIG. 22: Tumor growth kinetics in SUM190PT tumor-bearing mice treated with vehicle, zongertinib, T-DM1 or their combination at indicated doses and schedules. Median tumor volumes are plotted over time. Day 1 was the first day, day 21 the last day of the experiment.

FIG. 23: Waterfall plots on day 21 of change of tumor volume relative to day 1 in SUM190PT tumor-bearing mice treated with vehicle (not shown), zongertinib, T-DM1 or their combination at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

FIG. 24: Tumor growth kinetics in SUM190PT tumor-bearing mice treated with vehicle, zongertinib, capecitabine, trastuzumab or their combination at indicated doses and schedules. Median tumor volumes are plotted over time. Day 1 was the first day, day 21 the last day of the experiment.

FIG. 25: Waterfall plots on day 21 of change of tumor volume relative to day 1 in SUM190PT tumor-bearing mice treated with vehicle (not shown), zongertinib, capecitabine, trastuzumab or their combination at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

FIG. 26: Tumor growth kinetics in BT-474 tumor-bearing mice treated with vehicle, zongertinib, T-DXd, their dual combination or triple combination with pertuzumab at indicated doses and schedules. Median tumor volumes are plotted over time. Day 1 was the first day, day 42 the last day of the experiment.

FIG. 27: Waterfall plots on day 42 of change of tumor volume relative to day 1 in BT-474 tumor-bearing mice treated with vehicle (not shown), zongertinib, T-DXd, their dual combination or triple combination with pertuzumab at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

FIG. 28: Tumor growth kinetics in BT-474 tumor-bearing mice treated with vehicle, zongertinib, T-DM1 or their combination at indicated doses and schedules. Median tumor volumes are plotted over time. Day 1 was the first day, day 42 the last day of the experiment.

FIG. 29: Waterfall plots on day 42 of change of tumor volume relative to day 1 in BT-474 tumor-bearing mice treated with vehicle (not shown), zongertinib, T-DM1 or their combination at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

FIG. 30: Tumor growth kinetics in NCI-H2170 tumor-bearing mice treated with vehicle, zongertinib, T-DXd or their combination at indicated doses and schedules. Median tumor volumes are plotted over time. Day 1 was the first day, day 21 the last day of the experiment.

FIG. 31: Waterfall plots on day 21 of change of tumor volume relative to day 1 in NCI-H2170 tumor-bearing mice treated with vehicle (not shown), zongertinib, T-DXd or their combination at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

FIG. 32: Tumor growth kinetics in HCC1954 tumor-bearing mice treated with vehicle, zongertinib, T-DXd, their dual combination or triple combination with pertuzumab at indicated doses and schedules. Median tumor volumes are plotted over time. Day 1 was the first day, day 20 the last day of the experiment.

FIG. 33: Waterfall plots on day 20 of change of tumor volume relative to day 1 in HCC1954 tumor-bearing mice treated with vehicle (not shown), zongertinib, T-DXd, their dual combination or triple combination with pertuzumab combination at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

FIG. 34: Tumor growth kinetics in Calu-3 tumor-bearing mice treated with vehicle, zongertinib, trastuzumab, pertuzumab, combination of zongertinib with trastuzumab or pertuzumab or triple combination of zongertinib with trastuzumab and pertuzumab at indicated doses and schedules. Median tumor volumes are plotted over time. Day 1 was the first day, day 42 the last day of the experiment.

FIG. 35: Waterfall plots on day 42 of change of tumor volume relative to day 1 in Calu-3 tumor-bearing mice treated with vehicle (not shown), zongertinib, trastuzumab, pertuzumab, combination of zongertinib with trastuzumab or pertuzumab or triple combination of zongertinib with trastuzumab and pertuzumab at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

FIG. 36: Tumor growth kinetics in SK-OV-3 tumor-bearing mice treated with vehicle, zongertinib, T-DXd or their combination at indicated doses and schedules. Median tumor volumes are plotted over time. Day 1 was the first day, day 20 the last day of the experiment.

FIG. 37: Waterfall plots on day 20 of change of tumor volume relative to day 1 in SK-OV-3 tumor-bearing mice treated with vehicle (not shown), zongertinib, T-DXd or their combination at indicated doses and schedules. Each bar refers to one individual tumor. Bars were cut-off at 100%. Horizontal lines indicate the RECIST criteria (>20% progressive disease, 20% to −30% stable disease, <−30% partial response, 100% complete response).

### DESCRIPTION OF THE INVENTION

### Zongertinib

As used herein, zongertinib refers to the compound as defined below or to a pharmaceutically acceptable salt thereof:

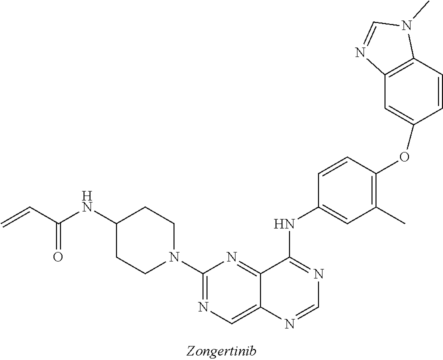 [SMILES: C=CC(=O)NC1CCN(c2ncc3ncnc(Nc4ccc(Oc5ccc6c(c5)ncn6C)c(C)c4)c3n2)CC1]

The IUPAC name of zongertinib is N-{1-[8-({3-methyl-4-[(1-methyl-1H-1,3-benzodiazol-5-yl)oxy]phenyl}amino)-[1,3]diazino [5,4-d]pyrimidin-2-yl]piperidin-4-yl}prop-2-enamide. In case of discrepancy between IUPAC name and depicted formula, the formula shall prevail. WO 2021/213800 discloses zongertinib as example compound I-01 and provides a procedure for its synthesis. Properties of zongertinib and evidence for inhibitory effect on HER2 wild-type and YVMA kinase activity, while sparing EGFR, are also disclosed in WO 2021/213800, which is herein incorporated by reference. Zongertinib as used herein also encompasses any tautomers, pharmaceutically acceptable salts, solid state forms of the compound, as well as solvates, including hydrates and solvates of pharmaceutically acceptable salts thereof.

In embodiments, zongertinib is a free base. Therefore, in any aspect or embodiment, the expression “zongertinib or a pharmaceutically acceptable salt thereof” can be replaced by “zongertinib”, without a reference to the pharmaceutically acceptable salt thereof. In embodiments, pharmaceutically acceptable salts of zongertinib are used. The term “pharmaceutically acceptable” used herein refers to compounds, materials, compositions and/or dosage forms which are, within the scope of sound medical judgement, suitable for use in contact with the tissues of human beings without excessive toxicity, irritation, allergic response, or other problem or complication, and commensurate with a reasonable benefit/risk ratio. As used herein, “pharmaceutically acceptable salts” of zongertinib refers to zongertinib wherein the compound is modified by making acid or base salts thereof. The term pharmaceutically acceptable salts as used herein generally includes both acid and base addition salts. Pharmaceutically acceptable acid addition salts refer to those salts which retain the biological effectiveness and properties of the free base and which are not biologically or otherwise undesirable, formed with inorganic acids or organic acids. Pharmaceutically acceptable base addition salts include salts derived from inorganic bases or organic nontoxic bases. Examples of pharmaceutically acceptable salts include, but are not limited to, mineral or organic acid salts of basic residues such as amines; alkali or organic salts of acidic residues such as carboxylic acids; and the like. For example, such salts include salts from benzenesulfonic acid, benzoic acid, citric acid, ethane sulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. In embodiments, pharmaceutically acceptable salts are selected from chloride and fumarate salts.

Pharmaceutically acceptable salts can be synthesized from zongertinib by conventional chemical methods. Generally, such salts can be prepared by reacting the free base form of zongertinib with a sufficient amount of the appropriate acid or base in water or in an organic diluent or solvent like ether, ethyl acetate, ethanol, isopropanol, or acetonitrile, or a mixture thereof.

The term “solvate” as used herein refers to an association or complex of one or more solvent molecules and zongertinib. Examples of solvents include water, isopropanol, ethanol, methanol, dimethyl sulfoxide (DMSO), ethyl acetate, acetic acid, tert-butyl methyl ether, tetrahydrofuran, methylethyl ketone, N-methylpyrrolidone and ethanolamine. The term “hydrate” refers to a complex where the solvent molecule is water.

In embodiments, zongertinib is in an amorphous form. In embodiments, zongertinib is in a crystalline form, for example in one of the crystalline forms described in WO 2024/133302, which is herein incorporated by reference in its entirety.

### Combination Partner

As used herein, “combination partner” is selected from the group consisting of an anti-HER2 antibody, an anti-HER2 antibody-drug conjugate and a combination thereof. In the context of the previous sentence, “a combination thereof” includes a combination of anti-HER2 antibodies (such as trastuzumab+pertuzumab), a combination of anti-HER2 antibody-drug conjugates (such as T-DXd+T-DM1) and a combination of at least one anti-HER2 antibody with at least one anti-HER2 antibody-drug conjugate (such as pertuzumab+T-DXd). The use of the singular “a” or “the” combination partner does not preclude that a combination thereof as defined herein may be used. Preferably, “combination partner” is an anti-HER2 antibody or an anti-HER2 antibody-drug conjugate. Therefore, the anti-HER2 antibody or anti-HER2 antibody-drug conjugate as defined herein can be referred to individually as “combination partner” or collectively as “combination partners”.

In one aspect of the combination, compounds for use, methods of treatment, uses, kits or pharmaceutical compositions as defined herein, the combination partner is an anti-HER2 antibody or an anti-HER2 antibody-drug conjugate.

In another aspect, the combination partner is an anti-HER2 antibody.

The term “antibody” which may be used interchangeably with “antibody molecule” encompasses various antibody structures, including but not limited to poly- or monoclonal, chimeric, humanized, human, mono-, bi- or multispecific antibodies, single chain antibodies, single domain antibodies, and fragmented antibodies (also referred to as antibody fragments), such as Fab, F(ab)₂, F(ab′)₂, Fab′, single chain variable-fragments (scFv) or antigen binding domains of an antibody, so long as they exhibit the desired antigen-binding activity. The term “antibody” shall encompass complete immunoglobulins as they are produced by lymphocytes and for example present in blood sera, monoclonal antibodies secreted by hybridoma cell lines, polypeptides produced by recombinant expression in host cells, which have the binding specificity of immunoglobulins or monoclonal antibodies, and molecules which have been derived from such immunoglobulins, monoclonal antibodies, or polypeptides by further processing while retaining their binding specificity. In particular, the term “antibody” includes complete immunoglobulins comprising two heavy chains and two light chains. The term further encompasses a fragment of an immunoglobulin, like Fab fragments and polypeptides having one or more variable domains derived from an immunoglobulin, like single chain antibodies (scFv), single domain antibodies, and the like.

The term antibody “fragment” shall refer to fragments of such an antibody retaining antigen binding capacities.

The antibody may have an effector function, such as ADCC or CDC, that is usually mediated by the Fc part (antibody constant region) of the antibody, or it may have no effector function, e.g. by lacking a Fc part or having a blocked, masked Fc part, in essence a Fc part that is not or insufficiently recognized by immune cells or immune system components, like the complement system.

The antibody or its fragment may be of any type, e.g. IgA, IgD, IgE, IgG, IgM. Preferred is IgG. The terms “monoclonal antibody” or “monoclonal antibody composition” as used herein refer to a preparation of antibody molecules of a single amino acid composition or of a homogenous antibody population, i.e., a homogeneous population consisting of a whole immunoglobulin, or a fragment or derivative thereof. Such antibody may be selected from the group consisting of IgA, IgD, IgE, IgG, IgM, or a fragment thereof.

A “recombinant antibody” is an antibody which has been produced by a recombinantly engineered host cell. It is optionally isolated or purified.

A “human antibody” is one which possesses an amino acid sequence which corresponds to that of an antibody produced by a human cell or derived from a non-human source that utilizes human antibody repertoires or other human antibody-encoding sequences. This definition of a human antibody specifically excludes a humanized antibody comprising non-human antigen-binding residues.

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies may have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

A “humanized” antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g. complementary determining regions (CDRs)) correspond to those of a non-human antibody, and all or substantially the entire FRs correspond to those of a human antibody. A humanized antibody optionally may comprise at least a portion of an antibody constant region derived from a human antibody. A “humanized form” of an antibody, e.g. a non-human antibody, refers to an antibody that has undergone humanization.

“Binding” of a polypeptide (such as an immunoglobulin, an antibody, or generally an antigen-binding molecule or a fragment thereof) means “having affinity for” or “having specificity for” a certain epitope, antigen or protein (or for at least one part, fragment or epitope thereof). The terms “binding” and “specific binding” referring to the binding of the antibody or antigen binding moiety to an epitope of the antigen may be determined in an in vitro assay, preferably in a plasmon resonance assay (BIAcore®, GE Healthcare Uppsala, Sweden) with purified wild-type antigen.

Generally, the term “specificity” refers to the number of different types of antigens or epitopes to which a particular antigen-binding molecule (such as an antibody described herein) can bind. The specificity of an antigen-binding molecule can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding protein: the lesser the value of the KD, the stronger the binding strength between an epitope and the antigen-binding molecule (alternatively, the affinity can also be expressed as the affinity constant (KA), which is 1/KD). As will be clear to the skilled person, affinity can be determined in a manner known in the art, depending on the specific antigen of interest. Avidity is the measure of the strength of binding between an antigen-binding molecule (such as an immunoglobulin, an antibody, or generally an antigen-binding molecule or a fragment thereof) containing it and the pertinent antigen. Avidity is related to both the affinity between an epitope and its antigen binding site on the antigen-binding molecule and the number of pertinent binding sites present on the antigen-binding molecule. An epitope is a region of an antigen that is bound by an antigen-binding molecule (such as an antibody described herein). The term “epitope” includes any polypeptide determinant capable of specific binding to an antibody or antigen binding moiety. In certain embodiments, epitope determinants include chemically active surface groupings of molecules such as amino acids, glycan side chains, phosphoryl, or sulfonyl, and, in certain embodiments, may have specific three dimensional structural characteristics, and/or specific charge characteristics. Conformational and non-conformational epitopes are distinguished in that the binding to the former but not the latter is lost in the presence of denaturing solvents.

The expressions “variable domain” or “variable region” or Fv as used herein denotes each of the pair of light and heavy chains which is involved directly in binding the antibody to the antigen. The variable domain of a light chain is abbreviated as “VL” and the variable domain of a heavy chain is abbreviated as “VH”. The variable light and heavy chain domains have the same general structure and each domain comprises four framework (FR) regions whose sequences are widely conserved, connected by three HVRs (or CDRs). The framework regions adopt a beta-sheet conformation and the CDRs may form loops connecting the beta-sheet structure. The CDRs in each chain are held in their three-dimensional structure by the framework regions and form together with the CDRs from the other chain the antigen binding site. The antibody's heavy and light chain CDR regions play a particularly important role in the binding specificity/affinity of the antibodies.

Within the context of this invention, reference to CDR's is based on the definition of Chothia (Chothia and Lesk, J. Mol. Biol. 1987, 196:901-917), together with Kabat (E. A. Kabat, T. T. Wu, H. Bilofsky, M. Reid-Miller and H. Perry, Sequence of Proteins of Immunological Interest, National Institutes of Health, Bethesda (1983)).

The term “constant domains” or “constant region” as used within the current application denotes the sum of the domains of an antibody other than the variable region. The constant region is not directly involved in binding of an antigen, but exhibits various effector functions.

The “constant domains” as used in the antibodies disclosed herein are preferably from human origin, which is from a constant heavy chain region of a human antibody of the subclass IgG1, IgG2, IgG3, or IgG4 and/or a constant light chain kappa or lambda region. Such constant domains and regions are well known in the state of the art and e.g. described by Kabat et al. (“Sequence of proteins of immunological interest”, US Public Health Services, NIH Bethesda, MD, Publication No. 91).

The “Fc part” of an antibody is not involved directly in binding of an antibody to an antigen, but exhibits various effector functions. A “Fc part of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG1, IgG2, IgG3, and IgG4, IgA1, and IgA2. According to the heavy chain constant regions the different classes of immunoglobulins are called α, δ, ε, γ and μ, respectively. The Fc part of an antibody is directly involved in ADCC (antibody-dependent cell-mediated cytotoxicity) and CDC (complement-dependent cytotoxicity) based on complement activation, C1q binding and Fc receptor binding. Complement activation (CDC) is initiated by binding of complement factor C1q to the Fc part of most IgG antibody subclasses. While the influence of an antibody on the complement system is dependent on certain conditions, binding to C1q is caused by defined binding sites in the Fc part. Such binding sites are known in the state of the art and described e.g. by Boackle, R. J., et al, Nature 282 (1979) 742-743; Lukas, T. J., et al, J. Immunol. 127 (1981) 2555-2560; Brunhouse, R., and Cebra, J. J., Mol. Immunol. 16 (1979) 907-917; Burton, D. R., et al, Nature 288 (1980) 338-344; Thommesen, J. E., et al, Mol. Immunol. 37 (2000) 995-1004; Idusogie, E. E., et al, J. Immunol. 164 (2000) 4178-4184; Hezareh, M., et al, J. Virology 75 (2001) 12161-12168; Morgan, A., et al, Immunology 86 (1995) 319-324; EP 0 307 434. Such binding sites are e.g. L234, L235, D270, N297, E318, K320, K322, P331 and P329 (numbering according to EU index of Kabat, E. A., see below). Antibodies of subclass IgG1, IgG2 and IgG3 usually show complement activation and C1q and C3 binding, whereas IgG4 do not activate the complement system and do not bind C1q and C3. The term “domain” (of a polypeptide or protein) as used herein refers to a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain.

The expression “anti-HER2 antibody” refers to an antibody that binds, in particular binds specifically, to the HER2 protein. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. In preferred embodiments, the expression “anti-HER2 antibody” is thus intended as a synonym and is replaceable with “inhibitory antibody binding specifically to HER2”. Anti-HER2 antibodies used for the treatment of cancer are typically monoclonal, although polyclonal antibodies are not excluded by the term. The anti-HER2 antibody can bind to one or more different epitopes on HER2. An anti-HER2 antibody can comprise one HER2 binding moiety or multiple HER2 binding moieties, such as two, three, four or more HER2 binding moieties. Preferably, the anti-HER2 antibody comprises one or two HER2 binding moieties. Any one or more anti-HER2 antibody(ies) can be used according to the invention. Preferably, the anti-HER2 antibody is selected from the group consisting of: trastuzumab, pertuzumab, margetuximab, zanidatamab, disitamab, anvatabart, hersintuzumab, anbenitamab and combinations thereof. Also preferably, the anti-HER2 antibody is selected from the group consisting of: trastuzumab, pertuzumab, margetuximab and combinations thereof. These names refer to the respective antibodies commonly known in the art with those names. The additional explanations hereinbelow are provided merely for the sake of clarity and are not intended to be limiting.

Trastuzumab, also known as Herceptin, is a humanized IgG1 monoclonal antibody that binds to HER2, in particular to its extracellular domain, especially to the HER2 domain that binds to another HER2 protein. The mechanism of action underlying the antitumour effect of trastuzumab has not yet been fully determined, and may in fact involve several different mechanisms. Trastuzumab may exert its effects by the activation of antibody-dependent cellular cytotoxicity, prevention of HER2 dimerization, the inhibition of the cleavage of the extracellular domain of HER2, interactions with signalling pathways, cell-cycle arrest during the G1 phase, induction of apoptosis or inhibition of angiogenesis. HER2 downregulation has also been suggested as a possible mechanism. Trustuzumab is disclosed in e.g., WO 92/22653, which is herein incorporated by reference.

Pertuzumab, also known as Perjeta, is a humanized IgG1 monoclonal antibody that binds to HER2, in particular to the extracellular domain of HER2, especially to the extracellular dimerization subdomain of HER2 receptor. Pertuzumab reduces HER2 intracellular signalling by preventing HER2 from forming heterodimers with other HER receptors, such as HER3. Pertuzumab is disclosed in e.g., U.S. Pat. Nos. 6,949,245 and 7,862,817 each of which is herein incorporated by reference. Margetuximab, also known as Margenza, is a mouse/human chimeric IgG1 monoclonal antibody that binds to HER2, in particular to its extracellular domain, especially to the HER2 domain that binds to another HER2 protein. Margetuximab is disclosed in e.g., U.S. Pat. No. 8,802,093 which is herein incorporated by reference.

Zanidatamab, also known as ZW25 or Ziihera, is a humanised, bispecific/biparatopic, IgG1-like, monoclonal antibody that binds to HER2, in particular to the juxtamembrane extracellular domain and the dimerisation domain of HER2. Zanidatamab is disclosed in e.g., WO 2015/077891 and Weisser N.

E., et al.; Nature Communications (2023) 14:1394, which are herein incorporated by reference. Hersintuzumab is a humanized IgG1 monoclonal antibody that binds to HER2, in particular to the extracellular domain of HER2, especially to an epitope that is distinct from those recognised by trastuzumab and pertuzumab. Hersintuzumab is disclosed in e.g., U.S. Pat. No. 10,167,342 which is herein incorporated by reference.

Anbenitamab, also known as KN026, is a bispecific antibody that binds to HER2, in particular to the extracellular domain of HER2, especially to two distinct epitopes of the extracellular dimerization domain of HER2. It comprises heavy chains of trastuzumab and pertuzumab with a common light chain. Anbenitamab is disclosed in e.g. WO 2016/110267 which is herein incorporated by reference.

In another aspect, the anti-HER2 antibody is an inhibitory antibody. The expression “inhibitory antibody” is intended as a synonym of and replaceable with “antagonistic antibody”. An “inhibitory antibody” or “antagonistic antibody” within the meaning of this invention is an antibody that inhibits the interaction of HER2 with its ligand(s) or receptor(s).

In another aspect, the anti-HER2 antibody binds specifically to HER2.

In another aspect, the anti-HER2 antibody binds specifically to an extracellular domain of HER2.

In another aspect, the anti-HER2 antibody is an inhibitory antibody binding specifically to HER2.

In another aspect, the anti-HER2 antibody is an inhibitory antibody binding specifically to an extracellular domain of HER2.

In another aspect, the combination partner is selected from the group consisting of trastuzumab, pertuzumab, margetuximab, zanidatamab, disitamab, anvatabart, hersintuzumab, anbenitamab and a combination thereof.

In another aspect, the anti-HER2 antibody is selected from the group consisting of trastuzumab, pertuzumab, margetuximab, zanidatamab, disitamab, anvatabart, hersintuzumab, anbenitamab and a combination thereof.

In another aspect, the combination partner is selected from the group consisting of trastuzumab, pertuzumab, margetuximab and a combination thereof.

In another aspect, the anti-HER2 antibody is selected from the group consisting of trastuzumab, pertuzumab, margetuximab and a combination thereof.

In another aspect, the combination partner is selected from the group consisting of trastuzumab, pertuzumab and a combination thereof.

In another aspect, the anti-HER2 antibody is selected from the group consisting of trastuzumab, pertuzumab and a combination thereof.

In another aspect, the combination partner is trastuzumab or pertuzumab.

In another aspect, the anti-HER2 antibody is selected from the group consisting of trastuzumab and pertuzumab.

In another aspect, the combination partner is a combination of anti-HER2 antibodies, such as a combination of trastuzumab and pertuzumab.

In another aspect, the combination partner is a combination of trastuzumab and pertuzumab. Thus, the invention provides the combination of zongertinib or a pharmaceutically acceptable salt thereof with trastuzumab and pertuzumab, such combination being suitable for the uses, methods, pharmaceutical compositions and kits as defined herein.

In another aspect, the anti-HER2 antibody is trastuzumab.

In another aspect, the anti-HER2 antibody is pertuzumab.

In another aspect, the combination partner is trastuzumab. Thus, the invention provides the combination of zongertinib or a pharmaceutically acceptable salt thereof with trastuzumab, such combination being suitable for the uses, methods, pharmaceutical compositions and kits as defined herein.

In another aspect, the combination partner is pertuzumab. Thus, the invention provides the combination of zongertinib or a pharmaceutically acceptable salt thereof with pertuzumab, such combination being suitable for the uses, methods, pharmaceutical compositions and kits as defined herein. In another aspect, the combination partner is an anti-HER2 antibody-drug conjugate.

The term “antibody drug conjugate” (also abbreviated herein as “ADC”), as used herein, is well known in the art and describes a group of therapeutics that combine the specificity of tumor-targeting binders, such as e.g. antibodies, with the potency of highly cytotoxic agents. Accordingly, such conjugates of cytotoxic and/or cytostatic agents to antibodies specific for tumor cells are powerful tools to specifically target cancer cells for their destruction. ADCs are well known in the art and have been reviewed, for example, in Dumontet et al. 2023 (Dumontet, C., Reichert, J. M., Senter, P. D. et al. Nat Rev Drug Discov 22, 641-661 (2023)).

The expressions “anti-HER2 antibody-drug conjugate” and “anti-HER2 ADC” refer to an anti-HER2 antibody conjugated to a cytotoxic drug, also called payload, optionally via a linker. The anti-HER2 antibody in the ADC can deliver the payload to cells expressing HER2, especially to cells with high levels of HER2.

In another aspect, the anti-HER2 antibody-drug conjugate comprises an anti-HER2 antibody that is an inhibitory antibody.

In another aspect, the anti-HER2 antibody-drug conjugate comprises an anti-HER2 antibody binding specifically to HER2.

In another aspect, the anti-HER2 antibody-drug conjugate comprises an anti-HER2 antibody binding specifically to an extracellular domain of HER2.

In another aspect, the anti-HER2 antibody-drug conjugate comprises an anti-HER2 antibody that is an inhibitory antibody binding specifically to HER2.

In another aspect, the anti-HER2 antibody-drug conjugate comprises an anti-HER2 antibody binding specifically to an extracellular domain of HER2.

In another aspect, the anti-HER2 antibody-drug conjugate comprises an anti-HER2 antibody selected from the group consisting of trastuzumab, pertuzumab, margetuximab, zanidatamab, disitamab, anvatabart, hersintuzumab and anbenitamab. In this aspect, trastuzumab and zanidatamab can be as defined above in any of the aspects concerning the anti-HER2 antibody.

In another aspect, the anti-HER2 antibody-drug conjugate comprises trastuzumab. In this aspect, trastuzumab can be as defined above in any of the aspects concerning the anti-HER2 antibody.

In another aspect, the combination partner is selected from the group consisting of trastuzumab deruxtecan, trastuzumab emtansine, trastuzumab rezetecan, trastuzumab duocarmazine, zanidatamab zovodotin, disitamab vedotin, anvatabart opadotin and a combination thereof.

In another aspect, the anti-HER2 antibody-drug conjugate is selected from the group consisting of trastuzumab deruxtecan, trastuzumab emtansine, trastuzumab rezetecan, trastuzumab duocarmazine, zanidatamab zovodotin, disitamab vedotin, anvatabart opadotin and a combination thereof. These names refer to the respective ADCs commonly known in the art with those names. The additional explanations hereinbelow are provided merely for the sake of clarity and are not intended to be limiting. In this aspect, trastuzumab and zanidatamab can be as defined above in any of the aspects concerning the anti-HER2 antibody.

In another aspect, the combination partner is selected from the group consisting of trastuzumab deruxtecan and trastuzumab emtansine. In this aspect, trastuzumab can be as defined above in any of the aspects concerning the anti-HER2 antibody.

In another aspect, the anti-HER2 antibody-drug conjugate is selected from the group consisting of trastuzumab deruxtecan and trastuzumab emtansine. In this aspect, trastuzumab can be as defined above in any of the aspects concerning the anti-HER2 antibody.

In another aspect, the anti-HER2 antibody-drug conjugate is trastuzumab deruxtecan. In another aspect, the anti-HER2 antibody-drug conjugate is trastuzumab emtansine.

In another aspect, the combination partner is trastuzumab deruxtecan. Thus, the invention provides the combination of zongertinib or a pharmaceutically acceptable salt thereof with trastuzumab deruxtecan, such combination being suitable for the uses, methods, pharmaceutical compositions and kits as defined herein.

In another aspect, the combination partner is trastuzumab emtansine. Thus, the invention provides the combination of zongertinib or a pharmaceutically acceptable salt thereof with trastuzumab emtansine, such combination being suitable for the uses, methods, pharmaceutical compositions and kits as defined herein.

Trastuzumab emtansine or ado-trastuzumab emtansine, also known as T-DM1 or Kadcyla, comprises the anti-HER2 antibody trastuzumab, a thioether linker, and the microtubule inhibitor emtansine or DM1—a derivative of maytansine—as cytotoxic payload. The International Nonproprietary Name (INN) “trastuzumab emtansine” is published in the WHO's INN Recommended List 65, which is herein incorporated by reference.

“Trastuzumab deruxtecan”, also known as T-DXd or Enhertu, comprises the anti-HER2 antibody trastuzumab, a linker, and the topoisomerase I inhibitor deruxtecan or DXd—a derivative of exatecan—as cytotoxic payload. The International Nonproprietary Name (INN) “trastuzumab deruxtecan” is published in the WHO's INN Recommended List 78, which is herein incorporated by reference.

Trastuzumab rezetecan, also known as SHR-A1811, comprises the anti-HER2 antibody trastuzumab conjugated to a DNA topoisomerase I inhibitor (SHR169265) via cleavable tetrapeptide-based linkers. Trastuzumab duocarmazine or [vic-]trastuzumab duocarmazine, also known as SYD985, comprises the anti-HER2 antibody trastuzumab and a cleavable linker-drug called valine-citrulline-seco-DUocarmycin-hydroxyBenzamide-Azaindole (vc-seco-DUBA).

Zanidatamab zovodotin, also known as ZW49, comprises the anti-HER2 bispecific/biparatopic antibody zanidatamab, a protease cleavable linker and the microtubule inhibitor auristatin at a drug-to-antibody ratio (DAR) of 2.

Disitamab vedotin, also known as Aidixi or RC48, comprises the anti-HER2 antibody disitamab (also known as hertuzumab), a histone cleavable linker and vedotin, i.e. monomethyl auristatin E (MMAE).

Anvatabart opadotin, also known as ARX788, comprises an anti-HER2 antibody conjugated to a a noncleavable Amberstatin (AS269) drug-linker with a DAR of 1.9.

The INNs as used herein are meant to also encompass all biosimilar molecules with the same structure, in particular comprising the same, or substantially the same, amino acid sequences as the originator antibody, including but not limited to those biosimilar antibodies authorized under 42 USC § 262 subsection (k) in the US and equivalent regulations in other jurisdictions. A “biosimilar” as used herein refers to an antibody (isolated or as part of and ADC) or antigen-binding fragment that has the same primary amino acid sequence as compared to a reference antibody (e.g., trastuzumab) and optionally, may have detectable differences in post-translation modifications (e.g., glycosylation and/or phosphorylation) as compared to the reference antibody (e.g., a different glycoform).

In another aspect, the combination partner is:
a combination of anti-HER2 antibodies;
a combination of anti-HER2 antibody-drug conjugates; or
a combination of at least one anti-HER2 antibody with at least one anti-HER2 antibody-drug conjugate.

In another aspect, the combination partner is:
trastuzumab+pertuzumab; or
trastuzumab deruxtecan+pertuzumab.

Thus, the invention provides the combination of zongertinib or a pharmaceutically acceptable salt thereof with trastuzumab deruxtecan and pertuzumab, such combination being suitable for the uses, methods, pharmaceutical compositions and kits as defined herein.

The combination of all aspects in respect of the nature of zongertinib or the pharmaceutically acceptable salt thereof as described herein with all aspects in respect of the nature of the combination partner as described herein results in specific combinations which shall all be deemed to be specifically disclosed and to be embodiments of the invention and of all combinations, compositions, kits, methods, uses and compounds for use described herein. Preferred embodiments are combinations of aspects of zongertinib or the pharmaceutically acceptable salt thereof in respect of the nature of zongertinib with aspects of the anti-HER2 ADC in respect of the nature of the combination partner.

### Dosing Schedule

In another aspect, zongertinib or the pharmaceutically acceptable salt thereof is administered daily.

In another aspect, zongertinib or the pharmaceutically acceptable salt thereof is administered orally, preferably as a tablet.

In another aspect, zongertinib or the pharmaceutically acceptable salt thereof is administered daily and orally, preferably as a tablet.

In another aspect, zongertinib or the pharmaceutically acceptable salt thereof is administered in a daily dose of at least 30 mg, preferably of at least 60 mg, preferably of at least 80 mg, preferably of at least 120 mg, preferably of at least 180 mg, preferably of at least 200 mg, preferably of at least 240 mg, preferably of at least 300 mg, preferably of at least 360 mg.

In another aspect, zongertinib or the pharmaceutically acceptable salt thereof is administered in a daily dose of at least 30 mg, preferably of at least 60 mg, preferably of at least 80 mg, preferably of at least 120 mg, preferably of at least 180 mg, preferably of at least 200 mg, preferably of at least 240 mg, preferably of at least 300 mg.

As used herein, “daily” means within a timeframe of 24 h. The expressions “daily dose” and “total daily dose” refer to the amount of active substance, i.e. zongertinib or the combination partner, which is administered within said timeframe of 24 h. The 24 h timeframe does not necessarily start at noon or midnight. Preferably, the daily dose is administered once or twice per day.

In another aspect, zongertinib or the pharmaceutically acceptable salt thereof is administered in a daily dose of 30 mg to 600 mg, preferably of 60 mg to 600 mg, preferably of 80 mg to 600 mg, preferably of 120 mg to 600 mg.

In another aspect, zongertinib or the pharmaceutically acceptable salt thereof is administered in a daily dose of 30 mg to 360 mg, preferably of 60 mg to 360 mg, preferably of 80 mg to 360 mg, preferably of 120 mg to 360 mg.

In another aspect, zongertinib or the pharmaceutically acceptable salt thereof is administered in a daily dose of 30 mg to 300 mg, preferably of 60 mg to 300 mg, preferably of 80 mg to 300 mg, preferably of 120 mg to 300 mg.

In another aspect, zongertinib or the pharmaceutically acceptable salt thereof is administered in a daily dose of 60 mg to 360 mg.

In another aspect, zongertinib or the pharmaceutically acceptable salt thereof is administered in a daily dose of 60 mg to 240 mg.

In another aspect, zongertinib or the pharmaceutically acceptable salt thereof is administered in a daily dose of 30 mg, 60 mg, 120 mg, 180 mg, 200 mg, 240 mg, 300 mg, 360 mg, 400 mg, 420 mg, 480 mg, 500 mg, 540 mg or 600 mg.

In another aspect, zongertinib or the pharmaceutically acceptable salt thereof is administered in a daily dose of 30 mg, 60 mg, 120 mg, 180 mg, 200 mg, 240 mg, 300 mg or 360 mg.

In another aspect, zongertinib or the pharmaceutically acceptable salt thereof is administered in a daily dose of 30 mg, 60 mg, 120 mg, 180 mg, 200 mg, 240 mg or 300 mg.

In another aspect, zongertinib or the pharmaceutically acceptable salt thereof is administered in a daily dose of 60 mg, 120 mg, 180 mg, 200 mg or 240 mg.

In another aspect, zongertinib or the pharmaceutically acceptable salt thereof is administered in a daily dose of 60 mg.

In another aspect, zongertinib or the pharmaceutically acceptable salt thereof is administered in a daily dose of 120 mg.

In another aspect, zongertinib or the pharmaceutically acceptable salt thereof is administered in a daily dose of 180 mg.

In another aspect, zongertinib or the pharmaceutically acceptable salt thereof is administered in a daily dose of 200 mg.

In another aspect, zongertinib or the pharmaceutically acceptable salt thereof is administered in a daily dose of 240 mg.

In another aspect, zongertinib or the pharmaceutically acceptable salt thereof is administered in a daily dose of 300 mg.

In another aspect, zongertinib or the pharmaceutically acceptable salt thereof is administered in a daily dose of 360 mg.

In another aspect, zongertinib or the pharmaceutically acceptable salt thereof is administered once or twice daily. This means that the daily dose, in particular the daily dose as defined in any aspect described herein, is administered either as a single dose (once daily) or is divided in two separate administrations, each administered at a different time point of the day (twice daily), i.e. there are two administrations within 24 h. In case of a twice daily administration, it is preferred that each of the two daily administrations of zongertinib corresponds to half the daily dose. When zongertinib is administered twice daily, the two separate administrations are preferably separated by a time interval of at least 1, 2, 3, 4, 5, 6, 7, 8, 9, 10, 11 or 12 hours, preferably of 4, 5, 6, 7, 8, 9, 10, 11 or 12 hours, still preferably of 8, 9, 10, 11 or 12 hours, also preferably of approximately 12 hours.

In another aspect, zongertinib or the pharmaceutically acceptable salt thereof is administered once or twice daily and orally, preferably as a tablet.

In another aspect, zongertinib or the pharmaceutically acceptable salt thereof is administered at least for 21 consecutive days. In further preferred embodiments, zongertinib or the pharmaceutically acceptable salt thereof is administered for 21 days multiplied by X, wherein X is a natural number equal or larger than 1.

In another aspect, zongertinib or the pharmaceutically acceptable salt thereof is administered once daily in a daily dose of 60 mg, 120 mg, 180 mg, 240 mg, 300 mg, 360 mg, 400 mg, 420 mg, 480 mg, 500 mg, 540 mg or 600 mg or zongertinib or the pharmaceutically acceptable salt thereof is administered twice daily in a daily dose of 30 mg, 60 mg, 120 mg, 200 mg, 300 mg, 360 mg, 400 mg, 420 mg, 480 mg, 500 mg, 540 mg or 600 mg.

In another aspect, zongertinib or the pharmaceutically acceptable salt thereof is administered once daily in a daily dose of 60 mg, 120 mg, 180 mg, 240 mg, 300 mg or 360 mg or zongertinib or the pharmaceutically acceptable salt thereof is administered twice daily in a daily dose of 30 mg, 60 mg, 120 mg, 200 mg, 300 mg or 360 mg.

In another aspect, zongertinib or the pharmaceutically acceptable salt thereof is administered once daily in a daily dose of 60 mg, 120 mg, 180 mg, 240 mg or 300 mg or zongertinib or the pharmaceutically acceptable salt thereof is administered twice daily in a daily dose of 30 mg, 60 mg, 120 mg, 200 mg or 300 mg.

In another aspect, zongertinib or the pharmaceutically acceptable salt thereof is administered once daily in a daily dose of 60 mg, 120 mg, 180 mg, 240 mg, 300 mg or 360 mg.

In another aspect, zongertinib or the pharmaceutically acceptable salt thereof is administered once daily in a daily dose of 60 mg, 120 mg, 180 mg or 240 mg.

In another aspect, zongertinib or the pharmaceutically acceptable salt thereof is administered once daily in a daily dose of 60 mg.

In another aspect, zongertinib or the pharmaceutically acceptable salt thereof is administered once daily in a daily dose of 120 mg.

In another aspect, zongertinib or the pharmaceutically acceptable salt thereof is administered once daily in a daily dose of 180 mg.

In another aspect, zongertinib or the pharmaceutically acceptable salt thereof is administered once daily in a daily dose of 200 mg.

In another aspect, zongertinib or the pharmaceutically acceptable salt thereof is administered once daily in a daily dose of 240 mg.

In another aspect, zongertinib or the pharmaceutically acceptable salt thereof is administered once daily in a daily dose of 300 mg.

In another aspect, zongertinib or the pharmaceutically acceptable salt thereof is administered once daily in a daily dose of 360 mg.

In another aspect, zongertinib or the pharmaceutically acceptable salt thereof is administered orally, preferably as a tablet, once daily in a daily dose of 60 mg, 120 mg, 180 mg, 240 mg, 300 mg, 360 mg, 400 mg, 420 mg, 480 mg, 500 mg, 540 mg or 600 mg or zongertinib or the pharmaceutically acceptable salt thereof is administered orally, preferably as a tablet, twice daily in a daily dose of 30 mg, 60 mg, 120 mg, 200 mg, 300 mg, 360 mg, 400 mg, 420 mg, 480 mg, 500 mg, 540 mg or 600 mg.

In another aspect, zongertinib or the pharmaceutically acceptable salt thereof is administered orally, preferably as a tablet, once daily in a daily dose of 60 mg, 120 mg, 180 mg, 240 mg, 300 mg or 360 mg or zongertinib or the pharmaceutically acceptable salt thereof is administered orally, preferably as a tablet, twice daily in a daily dose of 30 mg, 60 mg, 120 mg, 200 mg, 300 mg or 360 mg.

In another aspect, zongertinib or the pharmaceutically acceptable salt thereof is administered orally, preferably as a tablet, once daily in a daily dose of 60 mg, 120 mg, 180 mg, 240 mg or 300 mg or zongertinib or the pharmaceutically acceptable salt thereof is administered orally, preferably as a tablet, twice daily in a daily dose of 30 mg, 60 mg, 120 mg, 200 mg or 300 mg.

In another aspect, zongertinib or the pharmaceutically acceptable salt thereof is administered orally, preferably as a tablet, once daily in a daily dose of 60 mg, 120 mg, 180 mg, 240 mg, 300 mg or 360 mg.

In another aspect, zongertinib or the pharmaceutically acceptable salt thereof is administered orally, preferably as a tablet, once daily in a daily dose of 60 mg, 120 mg, 180 mg or 240 mg. In another aspect, zongertinib or the pharmaceutically acceptable salt thereof is administered orally, preferably as a tablet, once daily in a daily dose of 60 mg.

In another aspect, zongertinib or the pharmaceutically acceptable salt thereof is administered orally, preferably as a tablet, once daily in a daily dose of 120 mg.

In another aspect, zongertinib or the pharmaceutically acceptable salt thereof is administered orally, preferably as a tablet, once daily in a daily dose of 180 mg.

In another aspect, zongertinib or the pharmaceutically acceptable salt thereof is administered orally, preferably as a tablet, once daily in a daily dose of 200 mg.

In another aspect, zongertinib or the pharmaceutically acceptable salt thereof is administered orally, preferably as a tablet, once daily in a daily dose of 240 mg.

In another aspect, zongertinib or the pharmaceutically acceptable salt thereof is administered orally, preferably as a tablet, once daily in a daily dose of 300 mg.

In another aspect, zongertinib or the pharmaceutically acceptable salt thereof is administered orally, preferably as a tablet, once daily in a daily dose of 360 mg.

It is also possible to combine any of the above defined doses of zongertinib within the course of a treatment, for example by altering the daily dose. For instance, it is possible to increase or decrease a dose of 120 mg (once or twice daily) for subsequent administrations.

The combination partner may be administered according to the respective Label or Summary of Product Characteristics.

In one aspect, the combination partner is administered once about every 1 week, once about every 2 weeks, once about every 3 weeks, or once about every 4 weeks.

In another aspect, the combination partner is administered once every two weeks. “Once every two weeks” as used herein means that one administration of the combination partner takes place approximately every 14 days.

In another aspect, the combination partner is administered once every three weeks. “Once every three weeks” as used herein means that one administration of the combination partner takes place approximately every 21 days.

Depending on the administration route, e.g. in case of intravenous administration, the combination partner may have to be administered by a health care professional. Therefore, it may not be practical to strictly adhere to the posology of once every two or three weeks, e.g. in case of public holidays or patient unavailability. For this reason, the posology may be adapted at any time, for instance by anticipating or delaying administration of the combination partner. Preferably, said anticipating or delaying is by 2, 3, 4, 5, 6 or 7 days.

Therefore, the expression “once every two weeks” is intended to be read as and interchangeable with “once about every two weeks”, preferably “once every 7 to 21 days”, “once every 8 to 20 days”, “once every 9 to 19 days”, “once every 10 to 18 days”, “once every 11 to 17 days”, “once every 12 to 16 days” or “once every 13 to 15 days”. Likewise, the expression “once every three weeks” is intended to be read as and interchangeable with “once about every three weeks”, preferably “once every 14 to 28 days”, “once every 15 to 27 days”, “once every 16 to 26 days”, “once every 17 to 25 days”, “once every 18 to 24 days”, “once every 19 to 23 days” or “once every 20 to 22 days”.

The term “patient” as used herein refers to a human, e.g. a human suffering from, at risk of suffering from, or potentially capable of suffering from cancer.

In another aspect, the combination partner is administered subcutaneously or intravenously.

In another aspect, the combination partner is administered intravenously. Intravenous administration may take place e.g. by infusion. Infusions can last between about 30 and 120 minutes, preferably between about 30 and 90 minutes. The first infusion of the combination partner at the start of treatment can be longer than subsequent infusions. For example, the first infusion may last about 90 minutes and subsequent infusions may last about 30 minutes.

In another aspect, the combination partner is administered at a dose between about 0.1 mg and 20 mg per kg of the patient's body weight, preferably at a dose of approximately 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5 or 22 mg per kg of the patient's body weight.

In another aspect, the combination partner is administered at a dose between about 0.1 mg and 20 mg per kg of the patient's body weight, preferably at a dose of approximately 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg per kg of the patient's body weight.

In another aspect, the combination partner, is administered at a dose between about 1 mg and 9 mg per kg of the patient's body weight, preferably at a dose of approximately 1, 1.5, 2, 2.5, 3, 3.5, 4, 4.5, 5, 5.5, 6, 6.5, 7, 7.5, 8, 8.5 or 9±0.2 mg/kg, preferably of approximately 3.5 mg/kg, 5.5 mg/kg, 6 mg/kg, 6.5 mg/kg or 8 mg/kg±0.2 mg/kg.

In another aspect, the combination partner, in particular the anti-HER2 ADC, is administered at a dose between about 1 mg and 7 mg per kg of the patient's body weight, preferably at a dose of approximately 1, 1.5, 2, 2.5, 3, 3.5, 4, 4.5, 5, 5.5, 6, 6.5 or 7±0.2 mg/kg, preferably of approximately 3.5 mg/kg, 5.5 mg/kg or 6.5 mg/kg±0.2 mg/kg, preferably of approximately 3.5 mg/kg or 5.5 mg/kg±0.2 mg/kg.

In another aspect, the combination partner is administered at a dose between about 100 mg and 1000 mg, preferably at a dose of approximately 100, 150, 200, 250, 300, 350, 400, 450, 500, 550, 600, 650, 700, 750, 800, 850, 900, 950 or 1000 mg±20 mg, preferably of about 400 or 850 mg±20 mg. In another aspect, the combination partner is administered at a dose between about 100 mg and 400 mg, preferably at a dose of approximately 150, 200, 250, 300, 350 or 400 mg, preferably at a dose of approximately 200 mg.

In another aspect, the combination partner is administered subcutaneously or intravenously once every three weeks. In this aspect, the combination partner is preferably administered at the dose as defined in any of the previous aspects.

In another aspect, the combination partner is administered intravenously once every three weeks. In this aspect, the combination partner is preferably administered at the dose as defined in any of the previous aspects.

In another aspect, the combination partner is administered intravenously once every two weeks. In this aspect, the combination partner is preferably administered at the dose as defined in any of the previous aspects.

In another aspect, the combination partner is trastuzumab and is administered intravenously once every 3 weeks at a dose of 5 to 9 mg/kg±0.2 mg/kg, preferably at a dose of approximately 6 mg/kg or 8 mg/kg±0.2 mg/kg.

In another aspect, the combination partner is trastuzumab deruxtecan and is administered intravenously once every 3 weeks at a dose of 5 to 7 mg/kg±0.2 mg/kg, preferably at a dose of approximately 5.4 mg/kg or 6.4 mg/kg±0.2 mg/kg.

In another aspect, the combination partner is trastuzumab emtansine and is administered intravenously once every 3 weeks at a dose of 3 to 4 mg/kg±0.2 mg/kg, preferably at a dose of approximately 3.6 mg/kg±0.2 mg/kg.

In another aspect, the combination partner is zanidatamab and is administered intravenously once every 2 weeks at a dose of 18 to 22 mg/kg±1 mg/kg, preferably at a dose of approximately 20 mg/kg±1 mg/kg.

It is also possible to combine any of the above defined doses of the combination partner within the course of a treatment, for example by altering the daily dose. For instance, it is possible to increase or decrease a dose of 5.5 mg/kg for subsequent administrations.

In another aspect, zongertinib or the pharmaceutically acceptable salt thereof and the combination partner are administered within the same line of treatment.

In another aspect, the treatment comprises one day when both zongertinib or the pharmaceutically acceptable salt thereof and the combination partner are administered.

In another aspect, the treatment comprises cycles of 21 days.

In another aspect, the treatment comprises cycles of 21 days, wherein zongertinib or the pharmaceutically acceptable salt thereof is administered on each day of each cycle and/or the combination partner is administered once per cycle.

In another aspect, the treatment comprises cycles of 21 days, wherein zongertinib or the pharmaceutically acceptable salt thereof is administered on each day of each cycle and the combination partner is administered once per cycle.

Any aspect referring to the administration of zongertinib or the pharmaceutically acceptable salt thereof (e.g. referring to its daily dose, oral administration route, once or twice daily schedule, etc.) may be combined with any aspect referring to the combination partner (e.g. referring to its dose, administration route, posology, etc.) to provide further aspects of the invention.

For example, in another aspect, zongertinib or the pharmaceutically acceptable salt thereof is administered orally and/or the combination partner is administered subcutaneously or intravenously.

In another aspect, zongertinib or the pharmaceutically acceptable salt thereof is administered orally and the combination partner is administered subcutaneously or intravenously. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects.

In another aspect, zongertinib or the pharmaceutically acceptable salt thereof is administered daily and/or the combination partner is administered once every two or three weeks.

In another aspect, zongertinib or the pharmaceutically acceptable salt thereof is administered daily and the combination partner is administered once every two or three weeks. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects.

In another aspect, zongertinib or the pharmaceutically acceptable salt thereof is administered daily and/or the combination partner is administered once every three weeks.

In another aspect, zongertinib or the pharmaceutically acceptable salt thereof is administered daily and the combination partner is administered once every three weeks. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects.

In another aspect, zongertinib or the pharmaceutically acceptable salt thereof is administered once or twice daily and/or the combination partner is administered once every two or three weeks. In another aspect, zongertinib or the pharmaceutically acceptable salt thereof is administered once or twice daily and the combination partner is administered once every two or three weeks. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects. In another aspect, zongertinib or the pharmaceutically acceptable salt thereof is administered once or twice daily and/or the combination partner is administered once every three weeks.

In another aspect, zongertinib or the pharmaceutically acceptable salt thereof is administered once or twice daily and the combination partner is administered once every three weeks. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects.

In another aspect, zongertinib or the pharmaceutically acceptable salt thereof is administered orally and daily and/or the combination partner is administered subcutaneously or intravenously once every two or three weeks.

In another aspect, zongertinib or the pharmaceutically acceptable salt thereof is administered orally and daily and the combination partner is administered subcutaneously or intravenously once every two or three weeks. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects.

In another aspect, zongertinib or the pharmaceutically acceptable salt thereof is administered orally and daily and/or the combination partner is administered subcutaneously or intravenously once every three weeks.

In another aspect, zongertinib or the pharmaceutically acceptable salt thereof is administered orally and daily and the combination partner is administered subcutaneously or intravenously once every three weeks. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects.

In another aspect, zongertinib or the pharmaceutically acceptable salt thereof is administered orally and once or twice daily and/or the combination partner is administered subcutaneously or intravenously once every two or three weeks.

In another aspect, zongertinib or the pharmaceutically acceptable salt thereof is administered orally and once or twice daily and the combination partner is administered subcutaneously or intravenously once every two or three weeks. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects.

In another aspect, zongertinib or the pharmaceutically acceptable salt thereof is administered orally and once or twice daily and/or the combination partner is administered subcutaneously or intravenously once every three weeks.

In another aspect, zongertinib or the pharmaceutically acceptable salt thereof is administered orally and once or twice daily and the combination partner is administered subcutaneously or intravenously once every three weeks. In this and the previous aspect, zongertinib or the pharmaceutically acceptable salt thereof is preferably administered at the daily dose according to any of the previous aspects. In addition or in alternative, the combination partner is preferably administered at the dose according to any of the previous aspects.

### Combination Therapy

It is to be understood that the combinations, compositions, kits, methods, uses or compounds for use according to this invention may envisage the simultaneous, concurrent, sequential, successive, alternate or separate administration of the active agents or components. It will be appreciated that zongertinib or the pharmaceutically acceptable salt thereof and the combination partner can be formulated either together or independently. For example, zongertinib or the pharmaceutically acceptable salt thereof and the combination partner may be administered either as part of the same pharmaceutical composition/dosage form or, preferably, in separate pharmaceutical compositions/dosage forms.

Zongertinib or the pharmaceutically acceptable salt thereof and the combination partner thus may be formulated, alone or together, in suitable dosage unit formulations containing conventional non-toxic pharmaceutically acceptable carriers, excipients and/or vehicles appropriate for each route of administration. Typical pharmaceutical compositions for administering zongertinib or the pharmaceutically acceptable salt thereof and the combination partner, separately or jointly, include for example tablets, capsules, suppositories, solutions, e.g. solutions for injection and infusion, elixirs, emulsions or dispersible powders. Dosage forms and formulations of active ingredients are known in the art and further described herein. Preferably, zongertinib is administered in the form described in WO 2024/133289, which is herein incorporated by reference in its entirety. Also preferably, the combination partner is administered in the form described in the respective Label or Summary of Product Characteristics.

As used herein, “combination” or “combined” and grammatical variants thereof within the meaning of this invention include, but are not limited to, a product, product for use, use or method that results from the mixing or combining of more than one active agent, in particular zongertinib and the combination partner as defined herein. The expressions “combination”, “combined” and grammatical variants thereof comprise both fixed (e.g. pharmaceutical composition) and non-fixed (e.g. free) combinations (e.g. kits), products, products for use, uses and methods, such as e.g. the simultaneous, concurrent, sequential, successive, alternate or separate use of zongertinib and the combination partner as defined herein.

As used herein, “combination” or “combined” and grammatical variants thereof refer in particular to a combination that takes place within the same line of treatment. Preferably, in the products, products for use, uses and methods described herein, there is at least one day where both zongertinib, or a pharmaceutically acceptable salt thereof, and the combination partner are administered. The term “fixed combination” means that zongertinib and the combination partner as defined herein are both administered to a patient simultaneously in the form of a single entity or dosage form. The term “non-fixed combination” means that the active agents are both administered to a patient as separate entities or dosage forms either simultaneously, concurrently, sequentially, successively, alternatively or otherwise separately with no specific time limits.

The term “simultaneous” refers to the administration of both compounds/compositions at substantially the same time. This form of administration may also be referred to as “concomitant” administration. The term “concurrent” refers to administration of the active ingredients within the same general time period, for example on the same day(s) but not necessarily at the same time. The term “sequential” administration includes administration of one active ingredient during a first time period, for example over the course of a few hours, days or a week, using one or more doses, followed by administration of the other active ingredient during a second time period, for example over the course of a few hours, days or a week, using one or more doses. An overlapping schedule may also be employed, which includes administration of the active ingredients on different days over the treatment period, not necessarily according to a regular sequence. The term “successive” administration, alternatively, refers to an administration where the second administration step is carried out immediately once the administration of the first compounds has been finished. Alternate administration includes administration of one active ingredient during a time period, for example over the course of a few hours, days or a week, followed by administration of the other active ingredient during a subsequent period of time, for example over the course of a few hours, days or a week, and then repeating the pattern for one or more cycles, wherein the overall number of repeats depends on the chosen dosage regimen. Variations of these general administration forms may also be employed.

Accordingly, in another aspect, zongertinib or the pharmaceutically acceptable salt thereof and the combination partner are administered simultaneously, concurrently, sequentially, successively, alternately or separately.

Preferably, the combination of zongertinib or the pharmaceutically acceptable salt thereof and the combination partner(s) is administered simultaneously or sequentially, such that the concentration of the individual active ingredients in the body is high enough to exhibit a synergistic effect. Combination therapy according to the invention can occur with or without instructions for combined use. The two or more active ingredients may thus be administered entirely separately or be entirely separate pharmaceutical dosage forms. The individual active ingredients may be pharmaceutical compositions that are also sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active. It can refer to either a fixed combination in one dosage unit form, or a kit of parts for the combined administration where zongertinib or the pharmaceutically acceptable salt thereof and the combination partner may be administered independently at the same time or separately within time intervals, especially where these time intervals allow that the combination partners show a cooperative (synergistic) effect.

In another aspect, zongertinib or the pharmaceutically acceptable salt thereof and the combination partner are administered simultaneously.

In another aspect, zongertinib or the pharmaceutically acceptable salt thereof and the combination partner are administered concurrently.

In another aspect, zongertinib or the pharmaceutically acceptable salt thereof and the combination partner are administered sequentially.

In another aspect, zongertinib or the pharmaceutically acceptable salt thereof and the combination partner are administered successively.

In another aspect, zongertinib or the pharmaceutically acceptable salt thereof and the combination partner are administered alternately.

In another aspect, zongertinib or the pharmaceutically acceptable salt thereof and the combination partner are administered separately.

In another aspect, zongertinib or the pharmaceutically acceptable salt thereof is administered before the combination partner. Preferably, the treatment comprises one day when both zongertinib or the pharmaceutically acceptable salt thereof and the combination partner are administered and zongertinib or the pharmaceutically acceptable salt thereof is administered before the combination partner.

In another aspect, the combination partner is administered immediately after zongertinib or the pharmaceutically acceptable salt thereof. In this context, “immediately after” means, e.g. 30 minutes, 1 hour, 2 hours, 3 hours or 4 hours after.

In another aspect, zongertinib or the pharmaceutically acceptable salt thereof is administered after the combination partner. Preferably, the treatment comprises one day when both zongertinib or the pharmaceutically acceptable salt thereof and the combination partner are administered and zongertinib or the pharmaceutically acceptable salt thereof is administered after the combination partner. In another aspect, the combination partner is administered immediately before zongertinib or the pharmaceutically acceptable salt thereof. In this context, “immediately before” means, e.g. 30 minutes, 1 hour, 2 hours, 3 hours or 4 hours before.

It has been shown herein that there is a synergistic effect, defined as a greater than additive benefit, provided by co-administration of zongertinib or a pharmaceutically acceptable salt thereof and a combination partner as defined herein in the treatment of cancer. It is believed that, by acting simultaneously, superior inhibition of cancer can be achieved leading to improved cancer treatment outcomes, at least for cancers normally susceptible to treatment by anti-HER2 therapies.

In an embodiment, the combination produces a synergistic therapeutic effect as compared to the sole administration of zongertinib or the pharmaceutically acceptable salt thereof or the sole administration of an individual combination partner.

In an embodiment, the combination comprises the anti-cancer medicament and the combination produces a synergistic therapeutic effect as compared to the sole administration of:
zongertinib or the pharmaceutically acceptable salt thereof;
an individual combination partner; and/or
the anti-cancer medicament.

In an embodiment, the combination comprises the anti-cancer medicament and the combination produces a synergistic therapeutic effect as compared to the administration of:
zongertinib or the pharmaceutically acceptable salt thereof and an individual combination partner;
zongertinib or the pharmaceutically acceptable salt thereof and the anti-cancer medicament; and/or
an individual combination partner and the anti-cancer medicament.

Although any combination of doses may be used, typically doses of the combination partner and zongertinib or the pharmaceutically acceptable salt thereof, that provide a synergistic effect, or greater than additive benefit, are used. For example, doses of the combination partner may be selected to synergistically lower overall toxicity when administered with zongertinib or the pharmaceutically acceptable salt thereof, while maintaining substantially the same overall treatment effect on cancerous cells as observed when the anti-HER2 antibody or anti-HER2 antibody-drug conjugate is administered alone, and vice-versa. In another example, doses of the combination partner when administered synergistically with zongertinib or the pharmaceutically acceptable salt thereof, may be selected to produce substantially the same overall toxicity while synergistically increasing the treatment effect on cancerous cells as observed when the combination partner is administered alone; and vice-versa. Due to this synergistic behavior, the combination partner may be advantageously administered at doses lower than currently approved doses by co-administration with zongertinib or the pharmaceutically acceptable salt thereof according to the invention without substantially reducing the efficacy of the cancer treatment. This has the benefit of reducing toxicity of the combination partner. In addition, the toxicity of zongertinib or the pharmaceutically acceptable salt thereof being co-administered may be less due to either a lower required dose or improved toxicological properties; this has the effect of further lowering overall toxicity of the combination without compromising the overall treatment effect. The use of lower doses of the combination partner and/or zongertinib or the pharmaceutically acceptable salt thereof are, for example, at most about 80%, about 50%, about 40%, about 30%, about 20%, about 10%, about 5%, about 3%, about 2%, about 1%, about 0.75%, about 0.5%, about 0.25%, or about 0.1%, etc. of the dose of the combination partner and/or zongertinib or the pharmaceutically acceptable salt thereof used alone. At these doses, a synergistic effect in the treatment of cancerous cells may be observed.

### Additional Intervention

The combination of zongertinib, or a pharmaceutically acceptable salt thereof, with a combination partner as defined herein for use in the treatment and/or prevention of oncological and/or hyperproliferative diseases, in particular cancer, as described herein may be administered in combination with additional intervention selected from the group consisting of radiation, surgery, an additional therapeutic agent such as chemotherapy and a combination thereof.

In one aspect, the present invention relates to a use of the combination of zongertinib, or a pharmaceutically acceptable salt thereof, with a combination partner as defined herein as described herein in combination with a cytostatic and/or cytotoxic active substance and/or in combination with radiotherapy and/or in combination with surgery and/or in combination with chemotherapy and/or in combination with immunotherapy in the treatment and/or prevention of cancer.

For the treatment of diseases of oncological nature, anticancer agents may be combined with radiotherapy, e.g. irradiation treatment, and/or surgery. The combination of zongertinib, or a pharmaceutically acceptable salt thereof, with a combination partner as defined herein, as well as the relative compounds for use, methods of treatment, uses, pharmaceutical compositions and kits as described herein, can be used in combination with radiotherapy. For example, a cancer patient may receive radiotherapy before and/or after or simultaneously with receiving therapy with the combination of zongertinib, or a pharmaceutically acceptable salt thereof, with a combination partner as defined herein, as well as with the relative compounds for use, methods of treatment, uses, pharmaceutical compositions and kits as described herein.

In embodiments, the combination of zongertinib, or a pharmaceutically acceptable salt thereof, with a combination partner as defined herein, as well as the relative compounds for use, methods of treatment, uses, pharmaceutical compositions and kits as described herein, can be used as adjuvant therapy in combination with a surgical procedure. The combination of zongertinib, or a pharmaceutically acceptable salt thereof, with a combination partner as defined herein, the relative compounds for use, methods of treatment, uses, pharmaceutical compositions and kits described herein may be administered for the purpose of diminishing the size of a tumor before surgical procedure (referred to as pre-operative adjuvant chemotherapy or neoadjuvant therapy), or may be administered after a surgical procedure for the purpose.

In embodiments, the combination of zongertinib, or a pharmaceutically acceptable salt thereof, a combination partner as defined herein, the relative compounds for use, methods of treatment, uses, pharmaceutical compositions and kits as described herein, may be administered in combination with an additional anti-cancer medicament. In this context, the anti-cancer medicament is additional to zongertinib or the pharmaceutically acceptable salt thereof and to the anti-HER2 antibody and/or the anti-HER2 antibody-drug conjugate. The use of the singular “an” or “the” anti-cancer medicament does not preclude that more than one anti-cancer medicament may be used. As used herein, the term “anti-cancer medicament” has the meaning commonly attributed to it in the art and includes any agent that can inhibit, stop or delay the growth of a tumor, maintain or reduce the size of a tumor or even completely eradicate it. Without being limited thereto, anti-cancer medicaments include chemotherapy, immunotherapy, cytostatic and/or cytotoxic active substances, cell proliferation inhibitors, anti-angiogenic substances, steroids or immune modulators/checkpoint inhibitors and combinations thereof. Specific examples of anti-cancer medicaments are given below.

In embodiments, the combination of zongertinib, or a pharmaceutically acceptable salt thereof, with a combination partner as defined herein, the relative compounds for use, methods of treatment, uses, pharmaceutical compositions and kits as described herein may be administered in combination with a cytostatic and/or cytotoxic active substance and/or in combination with immunotherapy.

In embodiments, the combination of zongertinib, or a pharmaceutically acceptable salt thereof, with a combination partner as defined herein, as well as the relative compounds for use, methods of treatment, uses, pharmaceutical compositions and kits as described herein, may be used in combination with one or several other pharmacologically active substances such as state-of-the-art or standard-of-care compounds, such as e.g. cell proliferation inhibitors, anti-angiogenic substances, steroids or immune modulators/checkpoint inhibitors, and the like.

Pharmacologically active substances, in particular anti-cancer medicaments, which may be administered in combination with the combination of zongertinib, or a pharmaceutically acceptable salt thereof, with a combination partner as defined herein, as well as the relative compounds for use, methods of treatment, uses, pharmaceutical compositions and kits as described herein, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors), inhibitors are for example (anti-) growth factor antibodies, (anti-) growth factor receptor antibodies and tyrosine kinase inhibitors, such as for example cetuximab, gefitinib, afatinib, nintedanib, imatinib, lapatinib, bosutinib, bevacizumab, pertuzumab and trastuzumab); antimetabolites (e.g. antifolates such as methotrexate, raltitrexed, pemetrexed, pyrimidine analogues such as 5fluorouracil (5fluorineU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumor antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors, ERK inhibitors, KRAS inhibitors (e.g. KRAS G12C inhibitors), signalling pathway inhibitors (e.g. SOS1 inhibitors), FLT3 inhibitors, BRD4 inhibitors, IGF-1R inhibitors, TRAILR2 agonists, Bcl-xL inhibitors, Bcl-2 inhibitors, Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors, immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), T-cell engagers (e.g. bi-specific T-cell engagers (BiTEs®) like e.g. CD3×BCMA, CD3×CD33, CD3×CD19), PSMA×CD3), tumor vaccines, chemotherapeutic agents, chemoprotective and supportive agents such as eribulin, amifostin, anagrelid, clodronat, filgrastin, interferon, interferon alpha, leucovorin, procarbazine, levamisole, mesna, mitotane, pamidronate and porfimer.

In embodiments, the anti-cancer medicament is selected from the group consisting of hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide); aromatase inhibitors (e.g. anastrozole, letrozole, liarozole, vorozole, exemestane, atamestane); platinum derivatives (e.g. cisplatin, oxaliplatin, carboplatin); taxanes (such as paclitaxel, docetaxel); inhibitors of CDKs (especially of CDK4 or CDK6); and antimetabolites (e.g. antifolates such as methotrexate, raltitrexed, pemetrexed, pyrimidine analogues such as 5fluorouracil (5fluorineU), ribonucleoside and deoxyribonucleoside analogues, capecitabine and gemcitabine, purine and adenosine analogues such as mercaptopurine, thioguanine, cladribine and pentostatin, cytarabine (ara C), fludarabine). In embodiments, the anti-cancer medicament is selected from the group consisting of capecitabine, eribulin, docetaxel, pemetrexed, 5-fluorouracil, folonic acid, cisplatin and a combination thereof.

In embodiments, the anti-cancer medicament is capecitabine.

In embodiments, the combination partner is trastuzumab and the anti-cancer medicament is capecitabine. Thus, the invention provides the combination of zongertinib or a pharmaceutically acceptable salt thereof with trastuzumab and capecitabine, such combination being suitable for the uses, methods, pharmaceutical compositions and kits as defined herein.

The anti-cancer medicament may be administered according to the respective Label or Summary of Product Characteristics.

In another aspect, the anti-cancer medicament is capecitabine and is administered orally at a daily dose of 2500 to 6000 mg, preferably of 3000 to 5600 mg, preferably of approximately 3000, 3300, 3600, 4000, 4300, 4600, 5000, 5300 or 5600 mg. In particular, capecitabine can be administered orally twice daily at a daily dose of approximately 1250 mg/m² for 2 weeks, followed by a 1-week rest period given as 3-week cycles.

### Cancer

In embodiments, the cancer is one of the following, without being restricted thereto:
Cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma;
cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), appendix, genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas;
Gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), HER2 positive breast cancer, triple negative breast cancer, Paget's disease of the breast;

cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; nonfluorineunctional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
peripheral nervous system cancer;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (BchlorineL), chronic T-cell lymphocytic leukemia (TchlorineL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP).

All cancers/tumors/carcinomas mentioned above which are characterized by their specific location/origin in the body are meant to include both the primary tumors and the metastatic tumors derived therefrom. Preferably, the cancer as defined herein (including in any embodiment referring to e.g. cancer types) is metastatic, advanced, and/or unresectable.

All cancers/tumors/carcinomas mentioned above may be further differentiated by their histopathological classification:

Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma; Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

In some embodiments, the cancer is a solid tumor. In some embodiments, the cancer is manifested by at least one solid tumor.

In some embodiments, the cancer is selected from the group consisting of brain cancer, breast cancer, endocrine cancer, gastrointestinal cancer, gynecologic cancer, head and neck tumor, lung cancer, nervous system cancer, and skin cancer.

Preferably, said brain cancer is a glioblastoma or a glioma.

Preferably, said breast cancer is lobular breast cancer. In addition or in alternative, said breast cancer is preferably metastatic.

Preferably, said endocrine cancer is nerve sheath tumor, more preferably HER2 mutant nerve sheath tumor.

Preferably, said gastrointestinal cancer is selected from the group consisting of anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer and small bowel cancer. In addition or in alternative, said gastrointestinal cancer may be a gastrointestinal neuroendocrine tumor, preferably HER2 mutant. Still preferably, said gastrointestinal cancer is selected from the group consisting of gastric adenocarcinoma, gastroesophageal junction adenocarcinoma and esophageal adenocarcinoma, in particular metastatic gastric adenocarcinoma, metastatic gastroesophageal junction adenocarcinoma and metastatic esophageal adenocarcinoma.

Preferably, said gynecologic cancer is selected from the group consisting of cervical cancer, uterine cancer, endometrial cancer and ovarian cancer.

As used herein, “head and neck tumor” preferably refers to a head and neck cancer. Preferably, said head and neck tumor is a salivary gland cancer or tumor.

Preferably, said lung cancer is non-small cell lung cancer (NSCLC).

Preferably, said nervous system cancer is peripheral nervous system cancer, more preferably HER2 amplified peripheral nervous system cancer.

Preferably, said skin cancer is not a melanoma, i.e. non-melanoma skin cancer.

In some embodiments, the cancer is selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

In some embodiments, the cancer is HER2 overexpressed, HER2 amplified and/or HER2 mutant (in particular HER2 exon 20 mutant) cancer selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

In another aspect, the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In some embodiments, the cancer is HER2 overexpressed, HER2 amplified and/or HER2 mutant (in particular HER2 exon 20 mutant) cancer selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In further embodiments, the cancer is selected from cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer). In still further embodiments, the cancer is NSCLC. In still further embodiments, the cancer is HER2 exon 20 mutant NSCLC.

In another aspect, the cancer is selected from the group consisting of breast cancer, esophageal cancer, gastric cancer, gastroesophageal junction cancer, lung cancer and ovarian cancer. In another aspect, the cancer is selected from the group consisting of breast cancer, esophageal cancer, gastric cancer, gastroesophageal junction cancer, and lung cancer. Preferably, said lung cancer is non-small cell lung cancer (NSCLC).

In another aspect, said cancer is advanced, unresectable and/or metastatic.

In a further preferred embodiment, said cancer is advanced and metastatic. In a further preferred embodiment, when said cancer is metastatic, the metastases are located in the lung, lymph node, bone or liver. In a further preferred embodiment, the cancer is advanced cancer including metastases and the metastases are located in the lung, lymph node, bone or liver.

In a preferred embodiment the cancer is unresectable advanced cancer comprising solid tumors and solid metastases and the metastases are located in the lung, liver, lymph node-tissue or bone. In another aspect, said cancer is an adenocarcinoma.

In another aspect, the cancer or tumor comprises a HER2 aberration. This means that the cells of the cancer or tumor harbor an aberration of HER2. As used herein, the expressions “HER2 aberration”, “aberration of HER2” and grammatical variants thereof have the meaning commonly attributed to them in the art and include any variation or alteration in the HER2 protein or its encoding gene, such as: overexpression of the HER2 protein, amplification of the HER2-encoding gene, mutations in the HER2-encoding gene and/or in the HER2 protein (in particular non-synonymous mutations, somatic mutations, mutations in specific regions, e.g. in the tyrosine kinase domain, in exon 20, etc.) as well as gene rearrangements of HER2 and/or NRG1. When the cancer comprises a HER2 aberration, it can be referred to as HER2 aberrant. When the cancer comprises an overexpression of the HER2 protein, it can be referred to as HER2 overexpressed. When the cancer comprises an amplification of the HER2-encoding gene, it can be referred to as HER2 amplified. When the cancer comprises a mutation in the HER2-encoding gene and/or in the HER2 protein, it can be referred to as HER2 mutant. In another aspect, the cancer is HER2 overexpressed, HER2 amplified and/or HER2 mutant. In another aspect, the cancer comprises a mutation in the tyrosine kinase domain of HER2.

In another aspect, the cancer is HER2 overexpressed and/or HER2 amplified.

In another aspect, the cancer comprises a gene rearrangement of HER2 and/or NRG1.

The presence or absence of HER2 alterations, including overexpression, amplification or mutations, can be determined using methods known in the art.

“HER2 overexpressed” as used herein refers to a cancer comprising cells that express HER2 at levels detectable by immunohistochemistry (e.g. IHC 2+ and IHC 3+) and/or methods assaying ERBB2 messenger RNA.

“HER2 amplified” as used herein refers to a cancer comprising cells exhibiting more than 2, in particular more than 3, 4, 5, 6, 7, 8, 9 or 10, preferably more than 6, copies of the HER2 gene ERBB2. HER2 expression, gene copy number and amplification can be measured, for example, by determining nucleic acid sequencing (e.g., sequencing of genomic DNA or cDNA), measuring mRNA expression, measuring protein abundance, or a combination thereof. HER2 testing methods include immunohistochemistry (IHC), in situ hybridization-including fluorescence in situ hybridization (FISH), chromogenic in situ hybridization (CISH), ELISAs, and RNA quantification using techniques such as Reverse Transcription-Polymerase Chain Reaction (RT-PCR), microarray analysis and Next Generation Sequencing (NGS). HER2 expression in or on the cancer sample cells can be compared to a reference cell. The reference cell can be a non-cancer cell obtained from the same subject as the sample cell. The reference cell can be a non-cancer cell obtained from a different subject or a population of subjects.

When the cancer is HER2 overexpressed and/or HER2 amplified in or on a cell, the cancer can be referred to as being “HER2 positive”. The level of HER2 amplification or overexpression in HER2 positive cancers is commonly expressed as a score ranging from 0 to 3 (i.e., HER2 0, HER2 1+, HER2 2+, or HER2 3+), with higher scores corresponding to greater degrees of expression.

In an aspect, the cancer is HER2 positive.

Preferably, “HER2 overexpressed”, “HER2 amplified” and “HER2 positive” mean that the cancer comprises cells having an immunohistochemistry score of 2+ or 3+.

Preferably, “HER2 overexpressed”, “HER2 amplified” and “HER2 positive” mean that the cancer comprises cells having HER2 amplification defined by in situ hybridization.

In some embodiments, the HER2 status of the cancer, specifically of a sample cell within the cancer, is determined. The determination can be made before the combination treatment begins, during treatment, or after treatment has been completed. In some instances, determination of the HER2 status results in a decision to change therapy (e.g., switching to a different anti-HER2 antibody or anti-HER2 ADC or switching from another treatment method to a method of the present invention).

The sample cell can be obtained as a biopsy specimen, by surgical resection, or as a fine needle aspirate (FNA).

In some embodiments, the sample cell is determined to be HER2 positive when HER2 is expressed at a higher level in the sample cell compared to a reference cell. In some embodiments, the cell is determined to be HER2 positive when HER2 is overexpressed at least about 1.5-fold (e.g., about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more) compared to a reference cell. In particular embodiments, the cell is determined to be HER2 positive when HER2 is overexpressed at least about 1.5-fold compared to the reference cell.

In some embodiments, the sample cell is determined to be HER2 positive when the FISH or CISH signal ratio is greater than 2.

“HER2 mutant” as used herein refers to a cancer harbouring at least one mutation, i.e. an alteration in the nucleic acid sequence of the HER2-encoding gene and/or an alteration in the amino acid sequence of the HER2 protein, including but not limited to those listed below. Mutations can be found with any method known to the skilled person, such as molecular diagnostic methods including but not limited to Polymerase Chain Reaction (PCR), Single Strand Conformational Polymorphism (SSCP), Denaturing Gradient Gel Electrophoresis (DGGE), Heteroduplex analysis, Restriction fragment length polymorphism (RFLP), Next Generation Sequencing (NGS) and Whole Exome Sequencing.

In embodiments of said HER2 mutant cancer, the mutation is a non-synonymous mutation. As used herein, the term “non-synonymous” has the meaning commonly attributed to it in the art and in particular refers to a mutation in the nucleic acid sequence of the HER2-encoding gene that alters the amino acid sequence of the HER2 protein.

In embodiments of said HER2 mutant cancer, the mutation is a somatic mutation. As used herein, the term “somatic” has the meaning commonly attributed to it in the art and in particular refers to a mutation in the nucleic acid sequence of the HER2-encoding gene occurring in a cell other than a gamete, a germ cell or a gametocyte.

In embodiments of said HER2 mutant cancer, the mutation is a non-synonymous somatic mutation.

In embodiments of said HER2 mutant cancer, the mutation is a non-synonymous somatic mutation in the tyrosine kinase domain of HER2.

In embodiments of said HER2 mutant cancer, the mutation is in the tyrosine kinase domain of HER2, in particular in the exon 20 of HER2. In the latter case, the cancer can be referred to as HER2 exon 20 mutant.

As used herein, a cancer comprising a mutation in the tyrosine kinase domain of HER2 is a cancer where the cancer or tumor cells harbour at least one mutation in the tyrosine kinase domain of HER2, which ranges from amino acids 694 to 883 and/or exons 18 to 21.

“Cancer with HER2 exon 20 mutation” or “HER2 exon 20 mutant cancer” as used herein refers to a cancer where the cancer or tumor cells harbour at least one HER2 exon 20 mutation including but not limited to the mutations listed below.

ERBB2 (HER2) exon 20 encodes for a part of the kinase domain and ranges from amino acids 769 to 835. Every mutation, insertion, duplication or deletion within this region is defined as an exon 20 mutation including the following mutations: p.A772_G773insMMAY; p.Y772_A775_dup (YVMA); p.A775_G776insYVMA; p. Y772insYVMA; p.M774delinsWLV; p.A775_G776insSVMA; p.A775_G776insVVMA; p.A775_G776ins YVMS; p.A775_G776insC; p.A776_delinsVC; p.A776_delinsLC; p.A776_delins VV; p.A776_delinsAVGC; p.A776_delinsIC; p.A776_V777delinsCVC; p.V777_insE; p.V777_G778insV; p.V777_G778insC; p.V777_G778insCG; p.V777_S779dup; p. V777L; p.V777M; p.G778_P780dup (GSP); p.G778_S779insCPG; p.G778_S779insG; p.G776_delinsVC; p.G776_V777delinsAVGCV; p.G776delinsLC; p.G776_V777delinsAVCV; p.G776delinsVV; p.G776_V777insL; p.G776_V777insVGC; p.G776C; p.G776A; p.G776L; p.G776V; p.P780_Y781insGSP (“p.” is referring to the HER2 protein). In addition HER2 mutations exist outside of exon 20 including the following mutations: p.S310A; p.S310F; p.S310Y; p.R678Q; p.G727A; p.T733I; p.L755S; p.L755A; p.L755F; p.L755P; p.L755S; p.V842I; p.D769Y; p.D769H; p.R103Q; p.G1056S; p.I767M; p.L869R; p.L869R; p.T733I; p.T862A; p. V697L; p.R929W; p.D277H; p.D277Y; p.G660D (“p.” is referring to the HER2 protein).

Of these, examples of tyrosine kinase mutations include: p.G727A; p.T733I; p.L755S; p.L755A; p.L755F; p.L755P; p.L755S; p.V842I; p.D769Y; p.D769H; p.I767M; p.L869R; p.L869R; p.T733I; p.T862A; p.V697L.

In an aspect, the cancer is HER2 positive breast cancer, in particular advanced HER2 positive breast cancer or HER2 positive metastatic breast cancer, preferably advanced HER2 positive metastatic breast cancer. Preferably, in this embodiment, the combination as described herein is administered as first line of therapy. Still preferably, in this embodiment, the combination as described herein is administered as second, third or further line of therapy. Also preferably, in this embodiment, the combination partner is selected from the group consisting of trastuzumab deruxtecan, trastuzumab emtansine, trastuzumab, pertuzumab, trastuzumab+pertuzumab and trastuzumab deruxtecan+pertuzumab. In addition or in alternative, an anti-cancer medicament may be administered in this aspect, in particular capecitabine when the combination partner is trastuzumab.

In an aspect, the cancer is HER2 positive esophageal cancer, HER2 positive gastric cancer, or HER2 positive gastroesophageal junction cancer, in particular HER2 positive esophageal adenocarcinoma, HER2 positive gastric adenocarcinoma, or HER2 positive gastroesophageal junction adenocarcinoma, preferably metastatic HER2 positive esophageal adenocarcinoma, metastatic HER2 positive gastric adenocarcinoma, or metastatic HER2 positive gastroesophageal junction adenocarcinoma. Preferably, in this embodiment, the combination as described herein is administered as first line of therapy. Still preferably, in this embodiment, the combination as described herein is administered as second or further line of therapy. Also preferably, in this embodiment, the combination partner is trastuzumab deruxtecan. In an aspect, the cancer is HER2 mutant lung cancer, in particular HER2 exon 20 mutant lung cancer or HER2 mutant NSCLC, preferably HER2 exon 20 mutant NSCLC.

In an embodiment, the cancer is advanced, unresectable or metastatic NSCLC harbouring a HER2 mutation, wherein said HER2 mutation is in the tyrosine kinase domain. Preferably, in this embodiment, the combination as described herein is administered as first line of therapy. Still preferably, in this embodiment, the combination as described herein is administered as second or further line of therapy. Also preferably, in this embodiment, the combination partner is selected from the group consisting of trastuzumab deruxtecan, trastuzumab emtansine, trastuzumab, pertuzumab and trastuzumab+pertuzumab.

In another aspect, the cancer is resistant to treatment with the combination partner as defined herein. In another aspect, the cancer is resistant to treatment with the anti-HER2 antibody and/or with the anti-HER2 antibody-drug-conjugate as defined herein. In another aspect, the cancer is resistant to treatment with the anti-HER2 antibody or the anti-HER2 antibody-drug-conjugate as defined herein. In another aspect, the cancer is resistant to treatment with the anti-HER2 antibody as defined herein. In another aspect, the cancer is resistant to treatment with anti-HER2 antibody-drug-conjugate as defined herein. In another aspect, the cancer is resistant to single-agent treatment with the anti-HER2 antibody and/or the anti-HER2 antibody-drug-conjugate as defined herein. In another aspect, the cancer is resistant to single-agent treatment with the anti-HER2 antibody or the anti-HER2 antibody-drug-conjugate as defined herein. In another aspect, the cancer is resistant to single-agent treatment with the anti-HER2 antibody as defined herein. In another aspect, the cancer is resistant to single-agent treatment with the anti-HER2 antibody-drug-conjugate as defined herein. In these aspects, the resistance may be advantageously overcome by treatment with zongertinib, a pharmaceutically acceptable salt thereof, a combination of zongertinib or a pharmaceutically acceptable salt thereof and the combination partner, a combination of zongertinib or a pharmaceutically acceptable salt thereof and the anti-HER2 antibody, or a combination of zongertinib a pharmaceutically acceptable salt thereof and the anti-HER2 antibody-drug-conjugate, or any other combination defined herein.

Cancer showing/developing “resistance” or being/becoming “resistant” to a therapy as used herein includes a cancer which is not responsive and/or exhibits reduced ability of producing a significant response, e.g., partial response and/or complete response, to treatment with the anti-HER2 antibody or the anti-HER2 antibody-drug-conjugate. Resistance may be de novo (primary) resistance or acquired resistance which arises in the course of a treatment method. The term “acquired resistance” as used herein indicates that the cancer becomes resistant and/or substantially less responsive to the effects of the anti-HER2 antibody and/or the anti-HER2 antibody-drug-conjugate as defined herein after being exposed to it for a certain period of time.

Cancer may develop resistance to single-agent treatment with the anti-HER2 antibody and/or anti-HER2 antibody-drug conjugate. In case of resistance to the anti-HER2 antibody-drug conjugate, the mechanisms of resistance may be attributed to the antibody or the payload, and may be receptor-related, referring to target accessibility, or intracellular, such as referring to the regulation of the intracellular signalling pathways.

In an embodiment, the cancer is resistant to treatment with the payload of an anti-HER2 antibody-drug conjugate.

In an embodiment, the cancer is resistant to treatment with deruxtecan, emtansine, rezetecan, duocarmazine, zovodotin, vedotin and/or opadotin.

In an embodiment, the cancer is resistant to treatment with deruxtecan or emtansine.

A cancer which initially responded to an anti-HER2 antibody and/or an anti-HER2 antibody-drug conjugate can relapse and become resistant to the anti-HER2 antibody and/or anti-HER2 antibody-drug conjugate when the anti-HER2 antibody and/or anti-HER2 antibody-drug conjugate is no longer effective in treating a subject with the cancer, for example despite the administration of increased dosages.

The cancer may be recurrent, relapsed, resistant or refractory to one or more anti-HER2 antibody and/or anti-HER2 antibody-drug conjugate(s). Thus, the patients may have received previous anti-cancer therapies with one or more anti-HER2 antibody and/or one or more anti-HER2 antibody-drug conjugate, which have not completely cured the disease.

Cancer with relapse and/or with resistance to the anti-HER2 antibody and/or the anti-HER2 antibody-drug conjugate may be particularly amenable for single agent treatment with zongertinib, or a pharmaceutically acceptable salt thereof, or for combined treatment with the anti-HER2 antibody and/or the anti-HER2 antibody-drug and zongertinib, or a pharmaceutically acceptable salt thereof, such as for second or further line treatment cycles (optionally in further combination with one or more other anti-cancer agents), or as add-on combination or as replacement treatment.

Therefore, an aspect refers to zongertinib or a pharmaceutically acceptable salt thereof for use in the treatment and/or prevention of cancer, wherein the cancer is resistant to treatment with an anti-HER2 antibody and/or with an anti-HER2 antibody-drug conjugate. Another aspect refers to zongertinib or a pharmaceutically acceptable salt thereof for use in the treatment and/or prevention of cancer, wherein the cancer is resistant to treatment with an anti-HER2 antibody or an anti-HER2 antibody-drug conjugate. Another aspect refers to zongertinib or a pharmaceutically acceptable salt thereof for use in the treatment and/or prevention of cancer, wherein the cancer is resistant to treatment with an anti-HER2 antibody. Another aspect refers to zongertinib or a pharmaceutically acceptable salt thereof for use in the treatment and/or prevention of cancer, wherein the cancer is resistant to treatment with an anti-HER2 antibody-drug conjugate. In these aspects, zongertinib, the pharmaceutically acceptable salt thereof, the cancer, the anti-HER2 antibody and the anti-HER2 antibody-drug conjugate can each independently or together be as described in any of the aspects or embodiments disclosed herein. In addition or in alternative, in these aspects, zongertinib or the pharmaceutically acceptable salt thereof, the anti-HER2 antibody and/or the anti-HER2 antibody-drug conjugate can be administered according to the dosing schedules defined herein.

Another aspect refers to a pharmaceutical composition comprising zongertinib or a pharmaceutically acceptable salt thereof and a pharmaceutically acceptable excipient, for use in the treatment and/or prevention of cancer, wherein the cancer is resistant to treatment with an anti-HER2 antibody and/or with an anti-HER2 antibody-drug conjugate. Another aspect refers to a pharmaceutical composition comprising zongertinib or a pharmaceutically acceptable salt thereof and a pharmaceutically acceptable excipient, for use in the treatment and/or prevention of cancer, wherein the cancer is resistant to treatment with an anti-HER2 antibody or an anti-HER2 antibody-drug conjugate. Another aspect refers to a pharmaceutical composition comprising zongertinib or a pharmaceutically acceptable salt thereof and a pharmaceutically acceptable excipient, for use in the treatment and/or prevention of cancer, wherein the cancer is resistant to treatment with an anti-HER2 antibody. Another aspect refers to a pharmaceutical composition comprising zongertinib or a pharmaceutically acceptable salt thereof and a pharmaceutically acceptable excipient, for use in the treatment and/or prevention of cancer, wherein the cancer is resistant to treatment with an anti-HER2 antibody-drug conjugate. In these aspects, the pharmaceutical composition, zongertinib, the pharmaceutically acceptable salt thereof, the cancer, the anti-HER2 antibody and the anti-HER2 antibody-drug conjugate can each independently or together be as described in any of the aspects or embodiments disclosed herein. In addition or in alternative, in these aspects, zongertinib or the pharmaceutically acceptable salt thereof, the anti-HER2 antibody and/or the anti-HER2 antibody-drug conjugate can be administered according to the dosing schedules defined herein.

### Pharmaceutical Composition and Kit

It is provided a pharmaceutical composition comprising zongertinib or a pharmaceutically acceptable salt thereof, a combination partner and a pharmaceutically acceptable excipient, wherein the combination partner is selected from the group consisting of an anti-HER2 antibody, an anti-HER2 antibody-drug conjugate and a combination thereof. In embodiments, said pharmaceutical composition additionally comprises an anti-cancer medicament. Preferably, said pharmaceutical composition is for use in the treatment and/or prevention of cancer. Also provided herein is a method of treating and/or preventing cancer, the method comprising administering to a patient in need thereof said pharmaceutical composition, wherein optionally the pharmaceutical composition comprises a therapeutically effective amount of zongertinib or the pharmaceutically acceptable salt thereof and of the combination partner and of the anti-cancer medicament, if present. Also provided herein is a use of said pharmaceutical composition for the manufacture of a medicament for the treatment and/or prevention of cancer. In these aspects, zongertinib, the pharmaceutically acceptable salt thereof, the cancer, the anti-HER2 antibody and the anti-HER2 antibody-drug conjugate can each independently or together be as described in any of the aspects or embodiments disclosed herein. In addition or in alternative, in these aspects, zongertinib or the pharmaceutically acceptable salt thereof, the anti-HER2 antibody, the anti-HER2 antibody-drug conjugate and/or the anti-cancer medicament can be administered according to the dosing schedules defined herein.

The term “pharmaceutically acceptable excipient” refers to a non-toxic component that does not destroy the pharmacological activity of the compound with which it is formulated. Pharmaceutically acceptable excipients that may be used in the compositions include fillers, disintegrants, glidants, lubricants, and coating agents. The compositions may comprise further pharmaceutically acceptable excipients selected from buffers, dispersion agents, surfactants, wetting agents, emulsifiers, suspending agents, preservatives, antioxidants, opaquing agents, processing aids, colorants, sweeteners, perfuming agents, flavoring agents, diluents and other known additives usable in the manufacturing of a pharmaceutical product. Pharmaceutical compositions as referred to herein may contain conventional non-toxic pharmaceutically acceptable excipients.

Also provided herein is a pharmaceutical composition comprising zongertinib or a pharmaceutically acceptable salt thereof and a pharmaceutically acceptable excipient, for use in the treatment and/or prevention of cancer, wherein the pharmaceutical composition is administered in combination with a combination partner, wherein said combination partner is selected from the group consisting of an anti-HER2 antibody, an anti-HER2 antibody-drug conjugate and a combination thereof.

The terms “first”, “second” and “third” with respect to pharmaceutical compositions, as used herein, are solely intended to indicate that these compositions are two different compositions. Thus, these terms shall not be understood to refer to the order or sequence of administration.

In an embodiment of the kit as defined herein, the combination partner is an anti-HER2 antibody or an anti-HER2 antibody-drug conjugate.

In an embodiment of the kit as defined herein, the combination partner is an anti-HER2 antibody as defined herein.

In an embodiment of the kit as defined herein, the combination partner is anti-HER2 antibody-drug conjugate as defined herein.

In an embodiment of the kit as defined herein, the first and second pharmaceutical compositions are packaged together in a single container.

In an embodiment, the kit as defined herein additionally comprises a package insert. Preferably, the package insert comprises instructions. Still preferably, the instructions provide guidance on simultaneous, concurrent, sequential, successive, alternate or separate administration of zongertinib, its pharmaceutically acceptable salt or pharmaceutical composition and the combination partner.

In an embodiment, the kit is for use as a medicament.

In an embodiment, the kit is for use in the treatment and/or prevention of cancer.

In an embodiment, the kit further comprises instructions which specify a dose regimen for zongertinib or the pharmaceutically acceptable salt thereof as daily administration.

In an embodiment, the kit further comprises instructions specifying a daily dose of zongertinib as defined herein, in particular a daily dose of zongertinib or the pharmaceutically acceptable salt thereof of 60 mg to 360 mg.

In an embodiment, the kit further comprises instructions for administering the first and second pharmaceutical compositions concurrently.

In an embodiment, the kit further comprises instructions for administering the first, second and third pharmaceutical compositions concurrently.

In an embodiment, the kit further comprises instructions specifying a type of cancer to treat and the cancer is as defined herein, in particular the cancer is selected from the group consisting of breast cancer, esophageal cancer, gastric cancer, gastroesophageal junction cancer, lung cancer and ovarian cancer and/or the cancer is HER2 overexpressed, HER2 amplified and/or HER2 mutant cancer and/or the cancer is resistant to treatment with the combination partner.

In an embodiment, the kit further comprises instructions specifying at least one dosage that produces a synergistic therapeutic effect in a patient as compared to the sole administration of zongertinib or the pharmaceutically acceptable salt thereof, or to the sole administration of an individual combination partner.

In an embodiment, the kit further comprises instructions specifying at least one dosage regimen that produces a synergistic therapeutic effect in a patient as compared to the sole administration of:
zongertinib or the pharmaceutically acceptable salt thereof;
an individual combination partner; and/or
the anti-cancer medicament.

In an embodiment, the kit further comprises instructions specifying at least one dosage regimen that produces a synergistic therapeutic effect as compared to the administration of:
zongertinib or the pharmaceutically acceptable salt thereof and an individual combination partner;
zongertinib or the pharmaceutically acceptable salt thereof and the anti-cancer medicament; and/or
an individual combination partner and the anti-cancer medicament.

Also provided herein is a method of treating and/or preventing cancer, the method comprising administering to a patient in need thereof a kit comprising:

(i) a first pharmaceutical composition comprising zongertinib or a pharmaceutically acceptable salt thereof and a pharmaceutically acceptable excipient; and
(ii) a second pharmaceutical composition comprising a combination partner and a pharmaceutically acceptable excipient;
wherein the combination partner is selected from the group consisting of an anti-HER2 antibody, an anti-HER2 antibody-drug conjugate and a combination thereof. In an embodiment, the first pharmaceutical composition comprises a therapeutically effective amount of zongertinib or the pharmaceutically acceptable salt thereof and the second pharmaceutical composition comprises a therapeutically effective amount of the combination partner. In an aspect, the kit comprises a third pharmaceutical composition comprising an anti-cancer medicament and a pharmaceutically acceptable excipient, in particular a therapeutically effective amount of an anti-cancer medicament.

Also provided herein is a use of the kit as defined herein for the manufacture of a medicament for the treatment and/or prevention of cancer.

Features and advantages of the present invention will become apparent from the following detailed examples, which illustrate the principles of the invention by way of example without restricting its scope:

### Abbreviations

ADC Antibody drug conjugate
ATP Adenosine triphosphate
BID, bid twice daily (latin: bis in die)
CGI Cell Growth Inhibition
DX Deruxtecan
EGFR Epidermal growth factor receptor
ERBB erb-b receptor tyrosine kinase
ERBB2 erb-b2 receptor tyrosine kinase 2
FCS Fetal calf serum
HER2 Human epidermal growth factor receptor 2
HER3 Human epidermal growth factor receptor 3
HER4 Human epidermal growth factor receptor 4
i.v. intravenously
MAP Mitogen-activated protein
NSCLC Non-small cell lung cancer
PBS phosphate-buffered saline
QD, qd once daily (latin: quaque die)
qdx20 once daily for 20 days
qdx21 once daily for 21 days
q7d once weekly
q21d every 21st day, once every 3 weeks
q21dx1 once every 21 days for one administration
p.o. oral
PI3K Phosphoinositide 3-kinase
POC Percentage of Control
RPMI 1640 Roswell Park Memorial Institute (RPMI) 1640 formula medium
RTK Receptor tyrosine kinase
T-DM1 trastuzumab-DM1, ado-trastuzumab emtansine, Kadcyla
T-DXd trastuzumab deruxtecan, Enhertu
TGI Tumor growth inhibition
WT wild-type

### Example 1)—Combination of Zongertinib with T-DXd or T-DM1 in Xenografts Derived from NCI-N87 (Gastric Cancer Cell Line with HER2 Overexpression)

In this Example, the anti-tumor activity of zongertinib in combination with the ADCs T-DXd or T-DM1 is evaluated in a subcutaneous xenograft mouse model derived from the HER2-overexpressing human gastric cancer cell line NCI-N87 in NMRI-Foxn1^{nu} mice. It is shown that adding zongertinib enhances the antitumor activity of T-DXd and T-DM1.

### 1.1 Materials and Methods

### 1.1.1 Study Design

The study is performed in human gastric cancer cell line NCI-N87 grown as subcutaneous xenografts in NMRI-Foxn1^{nu} mice. For the combination with T-DXd, the study is designed as shown in Table 1. Day 1 was the first day, day 21 the last day of treatment and day 22 the final day of analysis. For the combination with T-DM1, the study is designed as shown in Table 2. Day 1 was the first day, day 21 the last day of treatment and the final day of analysis.

TABLE 1
Treatment groups for studying the combination of zongertinib
with T-DXd in xenografts derived from NCI-N87

| No. of | | Dose | | |
|---|---|---|---|---|
| mice | Treatment | [mg/kg] | Schedule | Route |
| 8 | Control | — | qdx21/q21dx1 | p.o./i.v. |
| 8 | Zongertinib | 20 mg/kg | qdx21 | p.o. |
| 8 | | 10 mg/kg | | |
| 8 | T-DXd | 3 mg/kg | q21dx1 | i.v. |
| 8 | Zongertinib + | 20 mg/kg + | qdx21 + | p.o. + |
| | T-DXd | 3 mg/kg | q21dx1 | i.v. |
| 8 | | 10 mg/kg + | | |
| | | 3 mg/kg | | |

TABLE 2
Treatment groups for studying the combination of zongertinib
with T-DM1 in xenografts derived from NCI-N87

| No. of | | Dose | | |
|---|---|---|---|---|
| mice | Treatment | [mg/kg] | Schedule | Route |
| 8 | Control | — | qdx21/q21dx1 | p.o./i.v. |
| 8 | Zongertinib | 20 mg/kg | qdx21 | p.o. |
| 8 | | 5 mg/kg | | |
| 8 | T-DM1 | 10 mg/kg | q21dx1 | i.v. |
| 8 | Zongertinib + | 20 mg/kg + | qdx21 + | p.o. + |
| | T-DM1 | 10 mg/kg | q21dx1 | i.v. |
| 8 | | 5 mg/kg + | | |
| | | 10 mg/kg | | |

### 1.1.2 Cells

NCI-N87 is a HER2-overexpressing human gastric cancer cell line that can be obtained from the American Type Culture Collection (ATCC #CRL-5822). Cells are cultured in T175 tissue culture flasks at 37° C. and 5% CO₂. The medium used is RPMI 1640 (ATCC #30-2001) supplemented with 10% fetal calf serum (FCS) (Thermo Scientific #SH 30084.03). Cultures are split twice weekly with a ratio of 1:2.

### 1.1.3 Mice

Mice are 6- to 10-week-old female NMRI-Foxn1^{nu} purchased from Taconic, Denmark. After arrival at the animal facility, mice are allowed to adjust to conditions at least for 5 days before they are used for the experiment. They are housed in Macrolon® type III cages in groups of 8 to 10 under pathogen-free, controlled and standardized conditions at 21.5±1.5° C. temperature, 55±10% humidity and 12-h light-dark cycle. Standardized diet (PROVIMI KLIBA) and autoclaved tap water are provided ad libitum. Subcutaneous microchips implanted under isoflurane anaesthesia are used to identify each mouse. Cage cards showing the study number, the animal identification number, the compound, the dose level, the administration route as well as the schedule remain with the animals throughout the study.

### 1.1.4 Establishment of Tumors and Randomization

To establish subcutaneous tumors, NCI-N87 cells are harvested by centrifugation, washed and resuspended in PBS+5% FCS at 2.5×10⁷ cells/ml. 100 μl cell suspension containing 2.5×10⁶ cells is injected subcutaneously into the right flank of the mice (1 site per mouse). Mice are randomly distributed between the treatment and the vehicle control group (11 days after cell injection) when tumors are well established and have reached volumes of 99 to 178 mm³.

### 1.1.5 Administration of Test Compounds

Zongertinib is suspended in 0.5% Natrosol and administered intragastrally by gavage needle with a volume of 10 ml/kg body weight. The suspension is used for a maximum of 7 days.

T-DM1 (Kadcyla) can be purchased from Roche. T-DXd (Enhertu) can be purchased from AstraZeneca. T-DM1 and T-DXd are diluted with saline (0.9%) and administered intravenously into the tail vein with a volume of 5 ml/kg.

Mice in the control group are treated with vehicle, i.e.:
(i) 0.5% Natrosol, administered once daily for the duration of the treatment intragastrally by gavage needle with a volume of 10 ml/kg body weight; and
(ii) saline (0.9%), administered once every three weeks intravenously into the tail vein with a volume of 5 ml/kg.

### 1.1.6 Monitoring Tumor Growth and Side Effects

Tumor volumes are measured three times a week with a caliper. The volume of each tumor [in mm³] is calculated according to the formula:

$$tumor volume = length \times {diameter}^{2} \times \frac{π}{6}$$

Median tumor volumes for each treatment group are plotted over time in the figures. The percent change from baseline is calculated as:

$$Change from baseline ( \% ) = ( tumor {volume}_{last day} / tumor {volume}_{1 st day} - 1 ) \times 100.$$

To monitor side effects of treatment, mice are inspected daily for abnormalities and body weight is determined three times a week. Animals are weighed and examined daily and sacrificed based on severity criteria, including body weight, tumor size and tumor necrosis and when the median tumor volume of the group reaches a size of approximately 540 mm³ for T-DXd or 504 mm³ for T-DM1. In addition, animals with tumor sizes exceeding 1500 mm³ in diameter, with ulcerating tumors or 18% body weight loss are euthanized for ethical reasons.

### 1.1.7 Statistical Analysis

The statistical evaluation of the tumor volume and the body weight is conducted on day 21 (last day of treatment) for T-DM1 and on day 22 for T-DXd.

In the results reported below relative to the combination with T-DXd, animal no. 8 (vehicle control) had to be euthanized on day 19 due to tumor necrosis and was therefore excluded from statistical evaluation.

For tumor volume absolute values are used. Due to the observed variation, nonparametric methods are applied.

For descriptive considerations the number of animals, the median, the minimum and the maximum of the tumor volume is calculated for each group.

For a quick overview of possible treatment effects, the median of the tumor volume of each treatment group T is referred to the median of the control C as tumor growth inhibition (TGI) defined as:

$$TGI = 100 \times \{ 1 - [ \frac{( {treated}_{final day} - {treated}_{day 1} )}{( {control}_{final day} - {control}_{day 1}} ] \}$$

For the body weight the percentage change referred to the initial weight of day 1 is used as target variable for the statistical analysis.

Due to the observed variation nonparametric methods are applied.

For the statistical evaluation two subtopics are of interest:
Monotherapy effects-comparison of the therapies versus the control group
Effect of the combination-comparison of combination versus monotherapies

One-sided non-parametric Mann-Whitney-Wilcoxon U-tests are applied to compare all treatment groups with the control, as well as the combination therapy group with the corresponding monotherapy groups, looking for
a decrease in the tumor volume (inhibition of tumor growth, efficacy parameter)
a decrease in the body weight change (body weight loss, tolerability parameter)

Within each subtopic the p values of the efficacy parameters are adjusted for multiple comparisons according to Bonferroni-Holm. The p values of the tolerability parameter remain unadjusted in order not to overlook a possible adverse effect.

The level of significance is fixed at α=5%. An adjusted p value of less than 0.05 is considered to show a statistically significant difference between the groups and differences are seen as indicative whenever 0.05≤p value<0.10.

In the results reported below relative to the combination with T-DM1, tumor growth after stop of treatment (day 21) was further evaluated. To do so, the time until the tumor reached a critical volume of 300 mm³ can be included as an additional evaluation parameter. If an animal is sacrificed for tumor necrosis without reaching the critical tumor volume, the time point is set to the last measurement time point and included as a censored observation.

For descriptive considerations, the number of animals, the median, the minimum and the maximum of the time to critical tumor volume is calculated.

The median survival time is derived from the Kaplan Meier survival curves per group as the time point with a survival probability (probability of reaching the critical tumor volume) of 0.5. For the treatment groups the median survival time is referred to the corresponding time of the control group as an estimate for the prolongation of the time to critical tumor volume by the therapy.

For the comparisons of the several groups a logrank test is applied.

The statistical evaluation is prepared using the software Graph Pad Prism.

### 1.2 Results

### 1.2.1 Combination with T-DXd

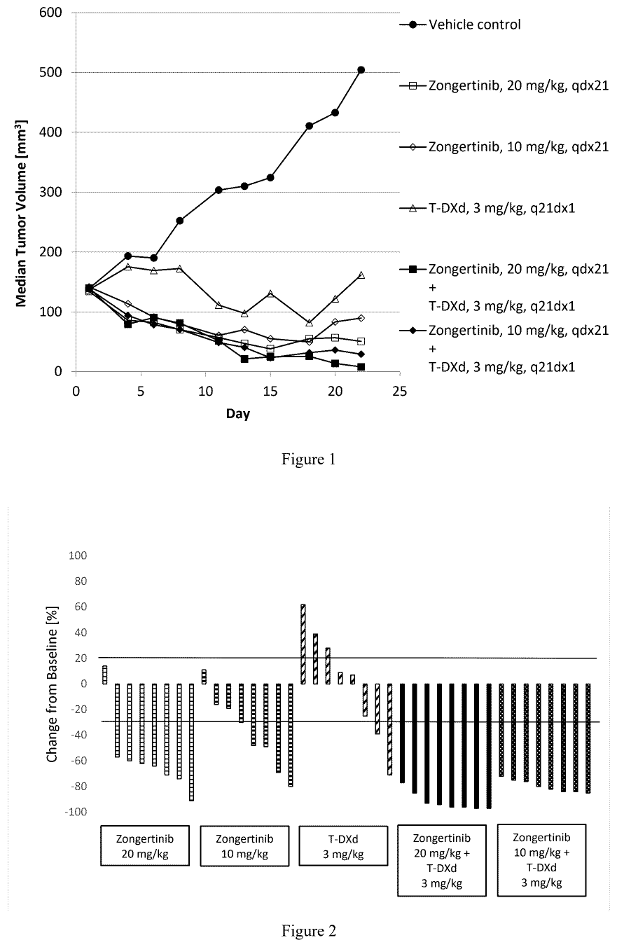

Results are summarised in Table 3 and in FIGS. 1 and 2.

TABLE 3
TGI at day 22 for each treatment group for studying the combination
of zongertinib with T-DXd in xenografts derived from NCI-N87

| | | | | p value | p value | p value |
|---|---|---|---|---|---|---|
| | Dose, | TGI | Regressions | vs. | vs. | vs. |
| Compound | schedule | [%] | >30% | control | * | * |
| Control | — qdx21/q21dx1 | | — | * | | |
| Zongertinib | 20 mg/kg, qdx21 | 123 | 7/8 | 0.0004 | 0.0005 | |
| | 10 mg/kg, qdx21 | 114 | 5/8 | 0.0004 | | 0.0002 |
| T-DXd | 3 mg/kg, q21dx1 | 93 | 2/8 | 0.0004 | 0.0004 | 0.0005 |
| Zongertinib + | 20 mg/kg, qdx21 + | 136 | 8/8 | 0.0004 | * | |
| T-DXd | 3 mg/kg, q21dx1 | | | | | |
| | 10 mg/kg, qdx21 + | 130 | 8/8 | 0.0004 | | * |
| | 3 mg/kg, q21dx1 | | | | | |

* indicates reference group for comparison
bold adjusted value <0.05

All treatments were well tolerated and showed no statistically different body weight gain, compared to the controls (data not shown).

Tumor growth inhibition of NCI-N87 xenografts was assessed following administration of T-DXd, zongertinib, and their combination (FIGS. 1 and 2).

Single treatment with T-DXd at 3 mg/kg once shrank tumors, but tumors started to regrow at the end of the 21-day experiment. Tumors in mice treated with zongertinib at 10 or 20 mg/kg/day continuously shrank more than those in mice treated with T-DXd. Combining T-DXd with either dosing schedule of zongertinib resulted in greater tumor shrinkage than any single treatment (FIGS. 1 and 2). In fact, the combination of 20 mg/kg or 10 mg/kg zongertinib with 3 mg/kg T-DXd showed significantly higher efficacy on tumor growth than corresponding monotherapies. Specifically, treatment with the combination of 20 mg/kg zongertinib with 3 mg/kg T-DXd was significantly more efficacious than 20 mg/kg zongertinib (median TGI=136% TGI versus 123% TGI, p=0.0005) and 3 mg/kg T-DXd (median TGI=136% TGI versus 93% TGI, p=0.0004) (FIG. 1 and Table 3). Similar results were obtained for the combination of 10 mg/kg zongertinib qd with 3 mg/kg T-DXd (FIG. 1 and Table 3).

### 1.2.2 Combination with T-DM1

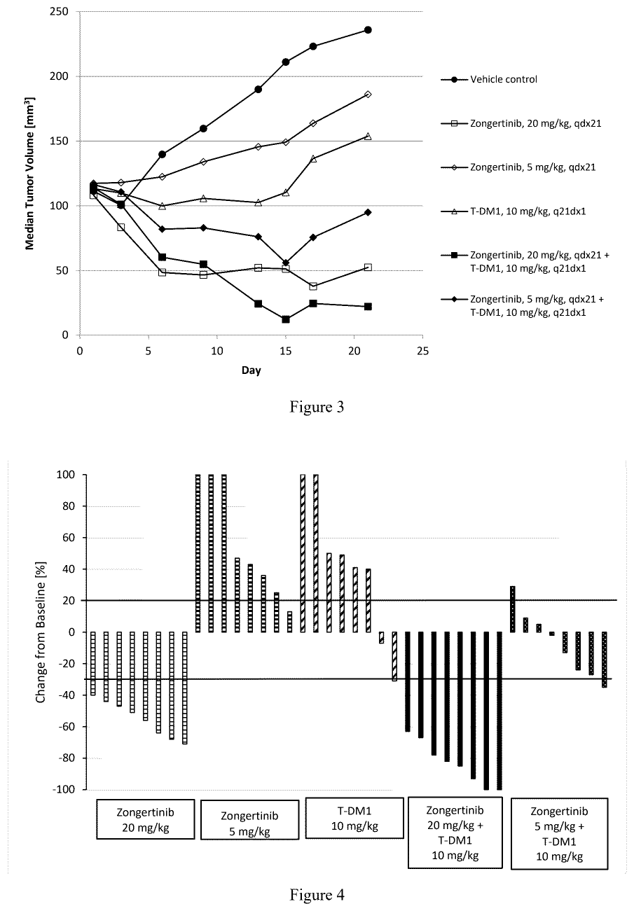

Results are summarised in Table 4 and in FIGS. 3 and 4.

TABLE 4
TGI at day 21 for each treatment group for studying the combination
of zongertinib with T-DM1 in xenografts derived from NCI-N87

| | | | | | | | Time | | |
|---|---|---|---|---|---|---|---|---|---|
| | | | | p value | p value | p value | to reach | p value | p value |
| | Dose, | TGI | Regressions | vs. | vs. | vs. | 300 mm³ | vs. | vs. |
| Compound | schedule | [%] | >30% | control | * | * | [Days] | * | * |
| Control | — qdx21/q21dx1 | | 0/8 | * | | | 30.0 | | |
| Zongertinib | 20 mg/kg, qdx21 | 145 | 8/8 | 0.0005 | 0.0037 | | 45.5 | 0.0241 | |
| | 5 mg/kg, qdx21 | 45 | 0/8 | 0.0415 | | 0.0018 | 30.0 | | 0.0016 |
| T-DM1 | 10 mg/kg, q21dx1 | 67 | 1/8 | 0.0104 | 0.0010 | 0.0103 | 39.5 | 0.0172 | 0.7551 |
| Zongertinib + | 20 mg/kg, qdx21 + | 174 | 8/8 | 0.0015 | * | | 60.5 | * | |
| T-DM1 | 10 mg/kg, q21dx1 | | | | | | | | |
| | 5 mg/kg, qdx21 + | 117 | 8/8 | 0.0005 | | * | 42.0 | | * |
| | 10 mg/kg, q21dx1 | | | | | | | | |

* indicates reference group for comparison
bold adjusted value <0.05

All treatments were well tolerated and showed no statistically different body weight gain, compared to the controls (data not shown).

T-DM1 at 10 mg/kg alone suppressed the growth of NCI-N87 tumors but did not shrink them. Zongertinib at 5 mg/kg/day also did not shrink tumors. Combining T-DM1 with zongertinib at 5 mg/kg/day reduced tumor size but tumors regrew after 15 days. Consistent with the previous example, treatment with zongertinib at 20 mg/kg/day induced deep and persistent tumor shrinkage. Intriguingly, adding T-DM1 at 10 mg/kg to the first dosing of zongertinib at 20 mg/kg/day induced an even deeper response (FIG. 3).

The combination of 20 mg/kg or 5 mg/kg zongertinib with 10 mg/kg T-DM1 showed significantly higher efficacy on tumor growth than corresponding monotherapies. Specifically, treatment with the combination of 20 mg/kg zongertinib with 10 mg/kg T-DM1 was significantly more efficacious than 20 mg/kg zongertinib (median TGI=174% TGI versus 145% TGI, p=0.0037) and 10 mg/kg T-DM1 (median TGI=174% TGI versus 67% TGI, p=0.0010) (FIG. 3 and Table 4). Similar results were obtained for the combination of 5 mg/kg zongertinib with 10 mg/kg T-DM1 (FIG. 3 and Table 4). After the stop of treatment at day 21, the outgrowth of tumors treated with the combination of 20 mg/kg zongertinib and 10 mg/kg T-DM1 was significantly delayed compared to both monotherapies (data not shown).

In conclusion, zongertinib is efficacious and tolerated in combination with HER2 targeting ADCs.

### Example 2)—Combination of Zongertinib with T-DM1 in Xenografts Derived from PC-9^{YVMA} (Non-Small Cell Lung Cancer with HER2^{YVMA} Mutation)

In the present Example, the anti-tumor activity of zongertinib in combination with T-DM1 is evaluated in a subcutaneous xenograft mouse model derived from the HER2 Exon20^{YVMA}-mutant human non-small cell lung cancer (NSCLC) cell line PC-9_YVMA-5 in NMRI-Foxn1^{nu} mice.

### 2.1 Materials and Methods

### 2.1.1 Study Design

The study is performed in human non-small cell lung cancer (NSCLC) cell line PC-9_YVMA-5 (PC-9 YVMA in short) grown as subcutaneous xenografts in NMRI-Foxn1^{nu} mice. The study is designed as shown in Table 5. Day 1 was the first day, day 21 the last day of treatment and day 24 the final day of analysis.

TABLE 5
Treatment groups for studying the combination of zongertinib
with T-DM1 in xenografts derived from PC-9 YVMA

| No. of | | Dose | | |
|---|---|---|---|---|
| mice | Treatment | [mg/kg] | Schedule | Route |
| 10 | Control | — | bid/q7d | p.o. |
| 8 | Zongertinib | 5 mg/kg | bid | |
| 8 | T-DM1 | 5 mg/kg | q7d | |
| 8 | Zongertinib + | 5 mg/kg | bid | |
| | T-DM1 | 5 mg/kg | q7d | |

### 2.1.2 Cells

PC-9 is a non-small cell lung cancer cell line that can be obtained from the European Collection of Authenticated Cell Cultures (ECACC #90071810) and expresses an EGFR DEL19 mutation. To generate the PC-9_YVMA-5 clone driven by HER2 YVMA, parental PC-9 cells are ectopically transduced to express the HER2 YVMA isoform and the EGFR alleles (mutant and wild type) are deleted using CRISPR technology. Cells are cultured in T175 tissue culture flasks at 37° C. and 5% CO₂. The medium used is RPMI 1640 supplemented with 10% FCS (Thermo Scientific #SH 30071.03), 0.5 μg/ml Puromycin (Gibco #A1113803) and 0.6 mg/ml Geneticin (Gibco #10131035). Cultures are split twice weekly with a ratio of 1:5-1:6.

### 2.1.3 Mice

Mice are treated as described in Example 1 (see paragraph 1.1.3).

### 2.1.4 Establishment of Tumors and Randomization

To establish subcutaneous tumors, PC-9_YVMA-5 cells are harvested by centrifugation, washed and resuspended in PBS+5% FCS at 5×10⁷ cells/ml. 100 μl cell suspension containing 5×10⁶ cells is injected subcutaneously into the right flank of the mice (1 site per mouse). Mice are randomly distributed between the treatment and the vehicle control group (12 days after cell injection) when tumors are well established and have reached volumes of 90 to 149 mm³.

### 2.1.5 Administration of Test Compounds

Zongertinib and T-DM1 are administered as described in Example 1 (see paragraph 1.1.5). Mice in the control group are treated with vehicle, i.e.:
(i) 0.5% Natrosol, administered twice daily for the duration of the treatment intragastrally by gavage needle with a volume of 10 ml/kg body weight; and
(ii) saline (0.9%), administered once weekly intravenously into the tail vein with a volume of 5 ml/kg.

### 2.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 370 mm³.

### 2.1.7 Statistical Analysis

The statistical evaluation of the tumor volume and the body weight is conducted on day 19 (last day of the controls). Apart from this, statistical analysis was performed as described in Example 1 (see paragraph 1.1.7), with no evaluation of tumor growth after stop of treatment.

### 2.2 Results

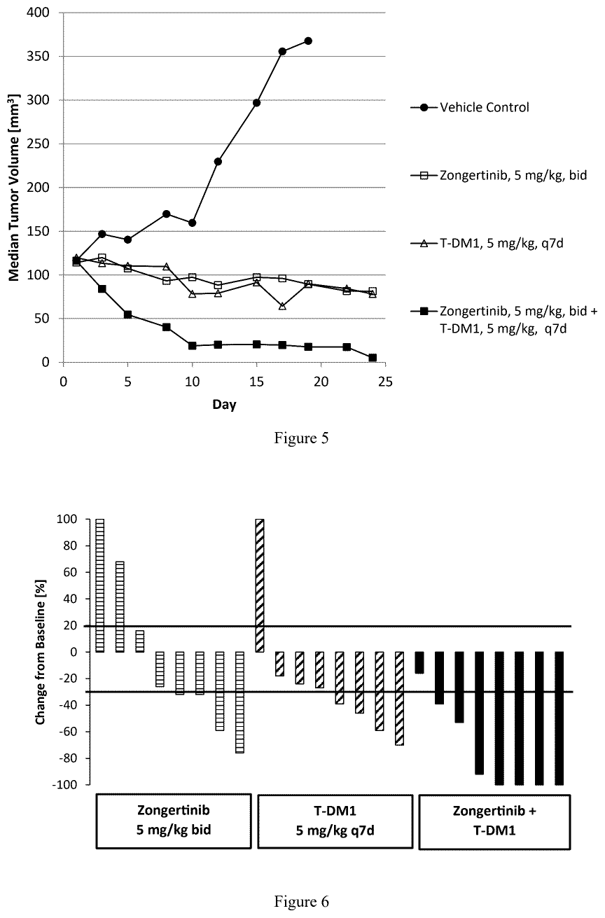

Results are summarised in Table 6 and in FIGS. 5 and 6.

TABLE 6
TGI at day 19 for each treatment group for
studying the combination of zongertinib with
T-DM1 in xenografts derived from PC9-YVMA

| | | | p value | p value | |
|---|---|---|---|---|---|
| | | TGI | vs. | vs. | Regressions |
| Compound | Dose, schedule | [%] | control | * | >30% |
| Control | — bid/q7d | | * | | — |
| Zongertinib | 5 mg/kg, bid | 110 | 0.0015 | 0.0070 | 4/8 |
| T-DM1 | 5 mg/kg, q7d | 112 | 0.0008 | 0.0070 | 4/8 |
| Zongertinib + | 5 mg/kg, bid | 139 | 0.0003 | * | 7/8 |
| T-DM1 | 5 mg/kg, q7d | | | | |

“*” indicates reference group for comparison
bold adjusted value <0.05

All treatments were well tolerated and showed no statistically different body weight gain, compared to the controls (data not shown).

In the mechanistic PC-9 EGFR^{KO}; HER2^{YVMA} tumor model, T-DM1 at 5 mg/kg/once weekly (q7d) or zongertinib at 5 mg/kg/day (monotherapies) abolished tumor growth whereas the combination of T-DM1 and zongertinib unexpectedly induced persistent tumor shrinkage (FIG. 5).

The combination of zongertinib with T-DM-1 showed significantly higher efficacy on tumor growth compared to both monotherapies and induced deeper tumor regressions. Specifically, treatment with the combination of zongertinib with T-DM1 was significantly more efficacious than single agent treatment with zongertinib (median TGI=139% versus 110%, p=0.0070) or with T-DM1 (median TGI=139% versus 112%, p=0.0008) (FIG. 5 and Table 6). 7 out of 8 tumors in the combination group went into regression of more than 30% (4 out of them regressed completely), while only 4 tumors each treated with zongertinib or T-DM1 went into regression of more than 30% and none of them regressed completely (Table 6 and FIG. 6).

### Example 3)—Combination of Zongertinib with T-DXd in Xenografts Derived from OE19 (Esophageal Cancer with HER2 Overexpression)

In this Example, the anti-tumor activity of zongertinib in combination with T-DXd is evaluated in a subcutaneous xenograft mouse model derived from the HER2-overexpressing human esophagus cancer cell line OE19 in NMRI-Foxn1^{nu} mice.

### 3.1 Materials and Methods

### 3.1.1 Study Design

The study is performed in human esophagus cancer cell line OE19 grown as subcutaneous xenografts in NMRI-Foxn1^{nu} mice. The study is designed as shown in Table 7. Day 1 was the first day, day 20 the final day of analysis.

TABLE 7
Treatment groups for studying the combination of zongertinib
with T-DXd in xenografts derived from OE19

| No. of | | Dose | | |
|---|---|---|---|---|
| mice | Treatment | [mg/kg] | Schedule | Route |
| 8 | Control | — | qdx20/q21dx1 | p.o./i.v. |
| 8 | Zongertinib | 20 mg/kg | qdx20 | p.o. |
| 8 | T-DXd | 3 mg/kg | q21dx1 | i.v. |
| 8 | Zongertinib + | 20 mg/kg + | qdx20 + | p.o. + |
| | T-DXd | 3 mg/kg | q21dx1 | i.v. |

### 3.1.2 Cells

OE19 is a HER2-overexpressing human esophagus cancer cell line that can be obtained from the European Collection of Authenticated Cell Cultures (ACACC Cat. No. 960071721). Cells are cultured in T75, T175 and 5-Layer tissue culture flasks at 37° C. and 5% CO₂. The medium used is RPMI 1640 (ATCC #30-2001) supplemented with 10% FCS (Thermo Scientific #SH 30084.03). Cultures are split twice weekly with a ratio of 1:3-1:4.

### 3.1.3 Mice

Mice are treated as described in Example 1 (see paragraph 1.1.3).

### 3.1.4 Establishment of Tumors and Randomization

To establish subcutaneous tumors, OE19 cells are harvested by centrifugation, washed and resuspended in PBS+5% FCS at 5×10⁷ cells/ml. 100 μl cell suspension containing 5×10⁶ cells is injected subcutaneously into the right flank of the mice (1 site per mouse). Mice are randomly distributed between the treatment and the vehicle control group (15 days after cell injection) when tumors are well established and have reached volumes of 67 to 204 mm³.

### 3.1.5 Administration of Test Compounds

Zongertinib, T-DXd and control are administered as described in Example 1 (see paragraph 1.1.5).

### 3.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 1190 mm³.

### 3.1.7 Statistical Analysis

The statistical evaluation of the tumor volume and the body weight is conducted on day 20. In the results reported below, animal no. 6 (vehicle control) had to be euthanized due to tumor size (day 15) and the last tumor volume was carried forward to day 20. In addition, animal no. 32 (20 mg/kg zongertinib+3 mg/kg T-DXd) had to be euthanized on day 18 due to severe body weight loss and was therefore excluded from statistical evaluation.

Apart from this, statistical analysis is performed as described in Example 1 (see paragraph 1.1.7), with no evaluation of tumor growth after stop of treatment.

### 3.2 Results

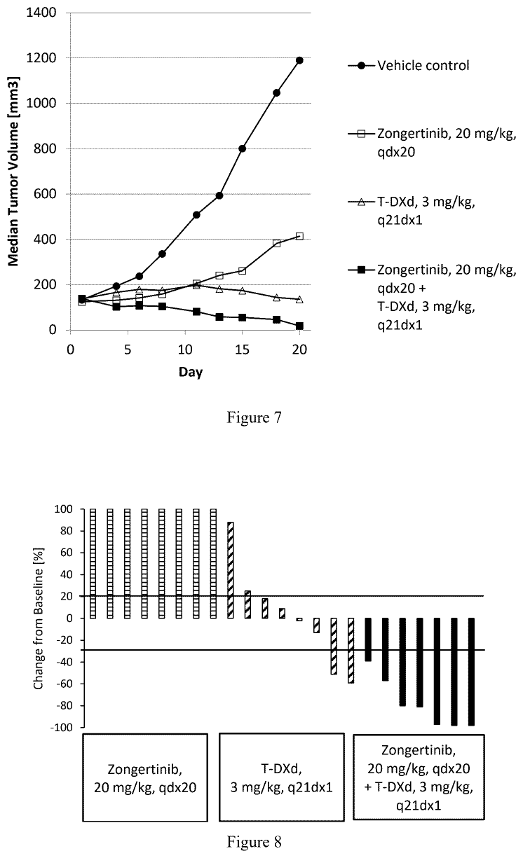

Results are summarised in Table 8 and in FIGS. 7 and 8.

TABLE 8
TGI at day 20 for each treatment group for studying the combination
of zongertinib with T-DXd in xenografts derived from OE19

| | | | p value | p value | Regres- |
|---|---|---|---|---|---|
| | | TGI | vs. | vs. | sions |
| Compound | Dose, schedule | [%] | control | * | >30% |
| Control | — qdx20/q21dx1 | | * | | — |
| Zongertinib | 20 mg/kg, qdx20 | 73 | 0.0002 | 0.0004 | 0/8 |
| T-DXd | 3 mg/kg, q21dx1 | 100 | 0.0002 | 0.0030 | 2/8 |
| Zongertinib + | 20 mg/kg, qdx20 + | 111 | 0.0002 | * | 7/7 |
| T-DXd | 3 mg/kg, q21dx1 | | | | |

“*” indicates reference group for comparison
bold adjusted value <0.05

Although one mouse treated with the combination of zongertinib and T-DXd had to be euthanized due to body weight loss and the group showed significantly lower body weight gain compared to the other groups, no severe body weight loss was observed in the other mice of this group, and therefore this combination treatment was judged as well tolerated. All treatments were well tolerated and showed no statistically different body weight gain, compared to the controls (data not shown). The combination of zongertinib with T-DXd showed significantly higher efficacy on tumor growth than corresponding monotherapies and induced deeper tumor regressions. Specifically, treatment with the combination of zongertinib with T-DXd was significantly more efficacious than zongertinib (median TGI=111% TGI versus 73% TGI, p=0.0004) and T-DXd (median TGI=111% TGI versus 100% TGI, p=0.0030) (Table 8 and FIG. 7). Remarkably, the combination treatment achieved persistent tumor shrinkage, even if neither monotherapy did. 7 out of 7 tumors in the combination group went into regression of more than 30%, including 3 complete responses, while only 2 tumors treated with T-DXd and no tumors treated with zongertinib went into regression of more than 30% (Table 8 and FIG. 8).

### Example 4)—Combination of Zongertinib with Trastuzumab, Pertuzumab or T-DXd in Breast Cancer Cell Lines

In this Example, the anti-proliferative activity of zongertinib in combination with T-DXd, trastuzumab or pertuzumab is tested in dose-titrations on three human breast cancer cell lines, BT-474, SK-BR-3 and MDA-MB-453. All three cell lines are ERBB2 wild type and express high levels of ERBB2.

### 4.1 Materials and Methods

### 4.1.1 Assay Description

The CellTiter-Glo 2.0 Assay (Promega, #G9243) provides a homogeneous method to determine the number of viable cells in culture by quantitating the amount of ATP present, which indicates the presence of metabolically active cells. Addition of CellTiter-Glo Luminescent Cell Viability Reagent results in cell lysis and generation of a luminescent signal that is proportional to the amount of ATP present. The amount of ATP is directly proportional to the number of cells present in culture.

### 4.1.2 Assay Conditions

Numbers of cells per well indicated in Table 9 are seeded in white, sterile CulturePlate-96 (white with white bottom, PerkinElmer, #6005680). In general, cells seeded on plates are incubated overnight in a humidified incubator at 37° C. and 5% CO₂, with the exception of MDA-MB-453, which grows at 37° C., but without CO₂.

TABLE 9
Cell culture conditions for the CellTiter-Glo Luminescent Cell Viability Assay

| | Number of | | |
|---|---|---|---|
| | cells per | | |
| Cell line | well | Supplier | Culture media |
| BT-474 | 3000 | ATCC | Hybri-Care Medium (ATCC, #46-X) + 10% FBS (Gibco, |
| | | HTB-20 | #26140-079) + 1.5 g/L sodium bicarbonate |
| | | | (Gibco, #25080-060) |
| MDA-MB- | 5000 | ATCC | Leibovitz's L-15 Medium (Gibco, #11415-049) + 10% FBS |
| 453 | | HTB-131 | (Gibco, #26140-079) |
| SK-BR-3 | 4000 | CLS | DMEM Medium (ATCC) (Sigma, #6429) + 1x Sodium |
| | | nr. 300333 | pyruvate (ThermoFisher Scientific, #11360088) + 10% FBS |
| | | | (Gibco, #26140-079) |

### 4.1.3 Addition of Test Compounds and Measurement

Compound start concentration and dilution steps are shown in Table 10.

TABLE 10
Start concentration and dilution steps for the
CellTiter-Glo Luminescent Cell Viability Assay

| | | BT-474 | SK-BR-3 | MDA-MB-453 |
|---|---|---|---|---|
| Zongertinib | start concentration | 0.01 μM | 0.025 μM | 0.1 μM |
| | dilution steps | 1:4 dilution | 1:4 dilution | 1:4 dilution |
| Trastuzumab | start concentration | 2.000 μg/mL | 5.000 μg/mL | 5.000 μg/mL |
| | dilution steps | 1:3 dilution | 1:3 dilution | 1:3 dilution |
| T-DXd | start concentration | 140.000 μg/mL | 0.190 μg/mL | 0.190 μg/mL |
| | dilution steps | 1:3 dilution | 1:3 dilution | 1:3 dilution |
| Pertuzumab | start concentration | 140.000 μg/mL | 140.000 μg/mL | 140.000 μg/mL |
| | dilution steps | 1:3 dilution | 1:3 dilution | 1:3 dilution |

Compounds (zongertinib, trastuzumab, T-DXd, pertuzumab) or DMSO are added to the cells in 3- or 4-fold serial dilution as detailed in Table 10 in duplicates.

At the time of test compound addition, a “time zero” (t=0) untreated cell plate is measured upon addition of CellTiter-Glo® Luminescent Cell Viability Reagent (Promega, #G9243). The plates are incubated for 15 minutes at room temperature to induce cell lysis and to stabilize the luminescent signal. The luminescent signal of each well is measured in an EnSpire or EnSight Multilabel Plate Reader (PerkinElmer).

After 5 days of incubation with test compounds, the cells of all other plates are measured upon addition of CellTiter-Glo® Luminescent Cell Viability Reagent (Promega, #G9243) to assess cell viability. The plates are incubated for 15 minutes at room temperature to induce cell lysis and to stabilize the luminescent signal. The luminescent signal of each well is measured in an EnSpire or EnSight Multilabel Plate Reader (PerkinElmer).

### 4.1.4 Data Analysis

The data can be analyzed with the Boehringer Ingelheim proprietary software MegaLab (curve fitting based on the program PRISM, GraphPad Inc.).

The CellTiter-Glo® assay output for vehicle-treated control cells after 5 days of incubation, corresponding to 100% cell viability, is taken as the reference signal for all subsequent calculations.

Relative cell viability in compound-treated cultures (signal percent of control, “POC”) can be calculated according to the following formula:

$${POC}_{( t = 120 h )} = 100 \times \frac{{luminescence}_{( compound wells )}}{{luminescence}_{( control wells )}}$$

In addition, for each compound-treated culture, the luminescent signal after incubation for 120 hours (POC_{(t=120 h)}) can be related to the signal at the start of treatment (POC_{(t=0 h)}) according to the following formula:

$${POC}_{( t = 0 h )} = 100 \times \frac{luminescence at t = 0 {h}_{( control wells )}}{luminescence at t = 120 {h}_{( control wells )}}$$

To calculate concentration-response curves, the POC data are analyzed using a four-parameter log-logistic function without any upper or lower limitation.

Relative cell growth inhibition (CGI %) in compound-treated cultures can be calculated according to the following formula:

$$\% {CGI}^{120 h} = \{ \begin{matrix}{S}_{t}^{120} \geq {S}_{c}^{0} : [ 1 - \frac{{S}_{t}^{120 h} - {S}_{c}^{0 h}}{{S}_{c}^{120 h} - {S}_{c}^{0 h}} ] \times 100 \% \\ {S}_{t}^{120} < {S}_{c}^{0} : [ 1 - \frac{{S}_{t}^{120 h} - {S}_{c}^{0 h}}{{S}_{c}^{0 h}} ] \times 100 \%\end{matrix}$$

St¹²⁰=POC_{(t=120 h)}
S_{c}⁰=POC_{(t=0 h)}

A CGI of >0% and <100% reflects a partial growth-inhibitory effect relative to vehicle-treated controls, a CGI of 100% is equivalent of complete blockade of growth, and a CGI of >100% is indicative of net cell death.

### 4.2 Results

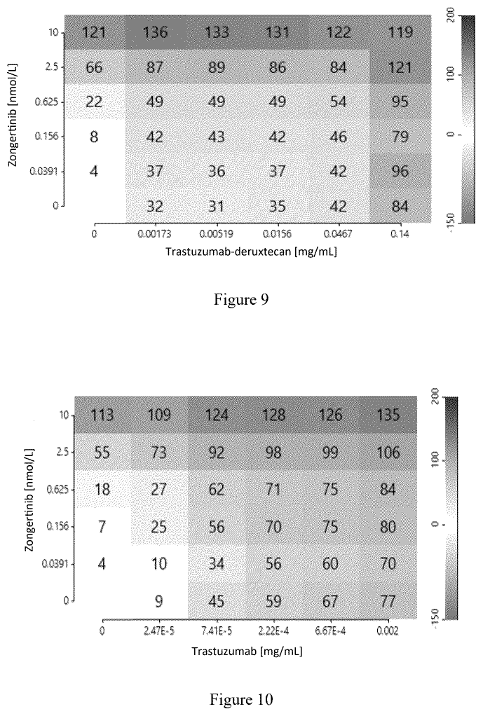

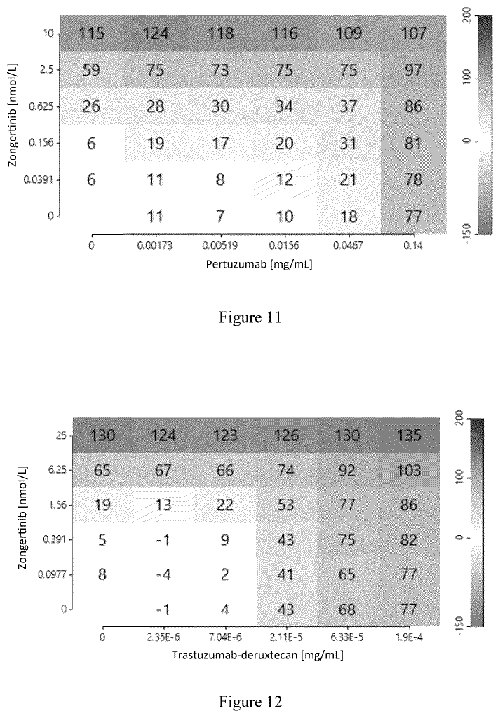

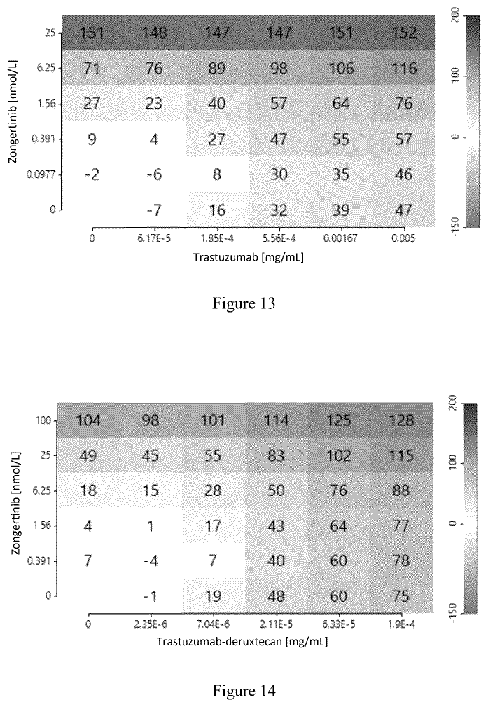

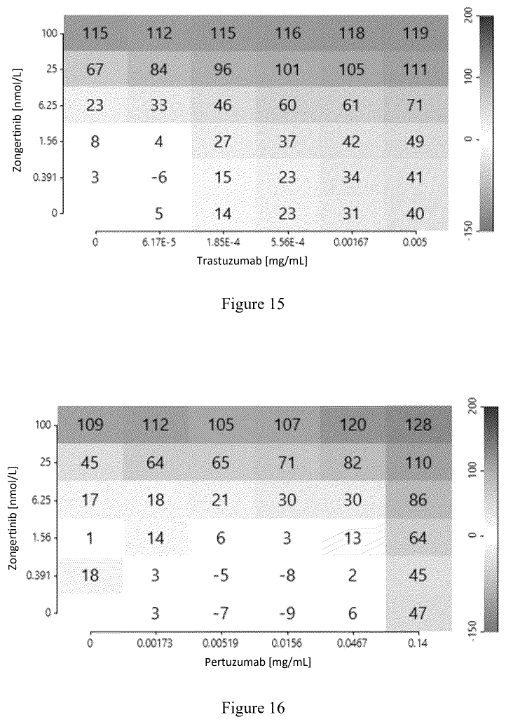

CGI for the combination of zongertinib with trastuzumab-deruxtecan, trastuzumab or pertuzumab in BT-474, SK-BR-3 and MDA-MB-453 cell lines are reported in FIGS. 9 to 16.

Within a certain concentration range, the data show combined anti-proliferative activity of zongertinib and trastuzumab-deruxtecan, trastuzumab or pertuzumab as seen by increasing cell growth inhibition (CGI) values, see e.g. the data for BT-474 for 2.5 nM zongertinib in combination with increasing dose levels for trastuzumab-deruxtecan, trastuzumab or pertuzumab (FIGS. 9, 10 and 11, respectively), the latter combination effect is slightly less prominent compared to the other two combination partners, trastuzumab-deruxtecan and trastuzumab, at the conditions tested.

These cellular combination data are better than individual treatment data.

The data are in line with the interpretation that a dose flexibility is given in combination of zongertinib with e.g. trastuzumab-deruxtecan, if e.g. safety aspects, bioavailability, PK/PD of either of the components needs to be considered for patient treatment.

### Example 5)—Efficacy of Zongertinib in NSCLC Cells Resistant to T-DXd

Zongertinib consistently inhibits downstream signaling of HER2 and the proliferation of cancer cells dependent on high expression of HER2WT. The inventors speculated that the differing mechanism of action of kinase inhibitors to antibody-based therapeutics could offer an opportunity to address resistance, which is not known or suggested for an EGFR WT sparing HER2 inhibitor such as zongertinib. Therefore, this Example investigates if zongertinib is efficacious in human cancer cells dependent on high expression of HER2WT but resistant to HER2 targeting ADCs.

### 5.1. Materials & Methods

### 5.1.1. Derivation of T-DXd Resistant Tumors In Vivo

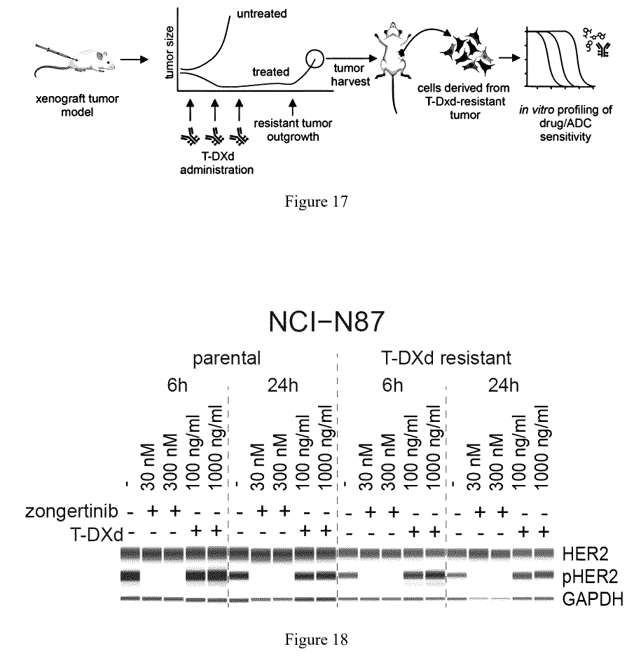

To generate T-DXd-resistant models, the NCI-N87 model is used and tumor-bearing mice are treated with three cycles of T-DXd, tumors that regrow are harvested and then tumor cells are cultivated in vitro, as shown in FIG. 17. Parental and T-DXd-resistant NCI-N87 cells are treated with deruxtecan (the T-DXd payload), T-DXd or zongertinib.

NCI-N87 cells are suspended at 2.5*10⁷ cells in PBS (Gibco, #14190-094) with 5% FBS (Gibco, #26140-079). The cell suspension is then injected subcutaneously in the right flank of BomTac: NMRI-Foxn1nu mice with a volume of 100 μl (2.5*10⁶ cells per mouse). After 15 days mice are randomized based on s.c. tumor volume at an average size of 120 mm³. At this day, treatment is started with 3 mg/kg T-DXd (Enhertu) i.v. From that point on, tumor sizes are measured three times weekly using a caliper. At day 22 after first treatment, a second treatment is injected at 10 mg/kg T-DXd. Treatments are followed on days 43; 64; 85 and 106 with 7 mg/kg i.v. Afterwards outgrowth is observed. Tumors are harvested on day 174 (#18 at 514.09 mm³) and day 209 (#17 at 626.21 mm³) and taken into culture in PBS on ice.

### 5.1.2. Tumors Expansion In Vitro

Tumors are cut into 1 mm³ pieces and homogenized using Tumor Dissociation Kit, mouse (Miltenyi Biotec, #130-096-730), in combination with the gentleMACS™ Octo Dissociator (Miltenyi). Cells are resuspended in 3 ml RPMI medium (PAN-Biotech, #P04-18047) supplemented with 10% FBS (Gibco, #26140-079), 1× GlutaMAX™ (Gibco, #35050-038), 1× Pen Strep (Gibco, #15140-122), and cell numbers are measured. 6.8*10⁶ cells are plated in 3 ml medium per well in a TC-treated 6 well cell culture plate (Corning, #3506) and grown in a humidified incubator at +37° C. and 5% CO₂. For the next 2 days cells are washed daily with 3 ml PBS (Gibco, #14190-094) to remove unattached cells and medium is replaced. Cells are passaged at 60-80% confluency by washing with 1 ml PBS, subsequently dissociating with 500 μL Trypsin (PAN-Biotech, #P10-0210300), and incubating cells at 37° C. until cells detach. Trypsin is blocked with 1 mL medium, and 500 μL of cell suspension is plated back to a 6 well plate with 3 ml medium or T25 cell culture flask with 7 ml medium (Corning, #353109). Cells are grown for 5 passages until no residual fibroblasts are visible before preforming proliferation assay upon drug treatment.

### 5.1.3. Proliferation Assay

Per well, 1000 cells are seeded in 40 μL medium in white, sterile 384-well culture plates (TC-treated; PerkinElmer #6007680) and incubated overnight in a humidified incubator at 37° C. and 5% CO₂. 0.1% DMSO-control or serial compound dilution in triplicates are added. T-DXd is diluted in 0.3% Tween solution and added to the cells. After 5 days of incubation with test compounds, the cells plates are measured upon addition of CellTiter Glo Luminescent Cell Viability Reagent (Promega, #G9243) to assess cell viability. The plates are placed on a shaker for five minutes and additionally incubated for 10 minutes at room temperature to induce cell lysis and to stabilize the luminescent signal. The luminescent signal of each well is measured in an EnSpire Multilabel Plate Reader 658 (PerkinElmer). The dose response curves are fit and visualized using Boehringer Ingelheim's proprietary software MegaLab and PRISM (GraphPad Inc.).

### 5.1.4. Immunoassay

Inhibition of HER2 phosphorylation in parental and T-DXd resistant NCI-N87 cells was analyzed with Simple Western (WES) capillary immunoassay. 100 000 cells were plated per well in 24 well format. Next day cells were treated with zongertinib or T-DXd for 6 h or 24 hours at concentrations indicated. Cells then were washed with 1 ml of PBS and lysed with 50 μL with RIPA lysis buffer (R2078, Sigma-Aldrich) supplemented with Halt™ proteinase inhibitor cocktail (1:100, Thermofisher Scientific, 78438) and 0.25 μL Benzonase nuclease (Millipore, 70746). 4 μL of lysate was taken for WES analysis using 12-230 kDa Separation Module (ProteinSimple, SM-W004). Antibodies used: HER2 (1:100, Cell Signaling, 4290), pHER2-Tyr1196 (1:100, Cell Signaling, 6942), GAPDH (1:300, Abcam, ab9485). WES data was analyzed using Compass for SW software (v6.1.0, ProteinSimple).

### 5.2. Results

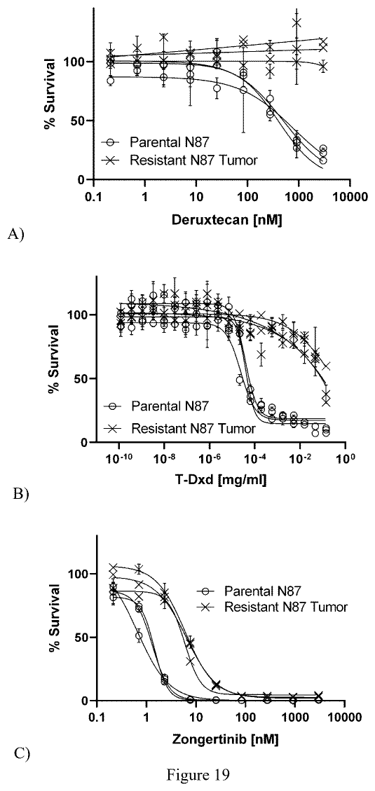

Using a cell line model, in this Example, it was demonstrated that zongertinib potently inhibits the proliferation of HER2-dependent cells that have acquired resistance to T-DXd, building on previous Examples, which demonstrate that zongertinib combines well with HER2 directed ADCs. Inhibition of HER2 phosphorylation in both parental and T-DXd resistant NCI-N87 cells was observed with zongertinib, but not T-DXd (FIG. 18). Parental NCI-N87 cells were sensitive to both T-DXd and deruxtecan whereas T-DXd-resistant cells were resistant to both T-DXd and deruxtecan, with at least 100-fold difference in their IC₅₀ (FIGS. 19A and 19B). Surprisingly, both parental and resistant NCI-N87 cells were sensitive to zongertinib, with less than 10-fold difference in their IC₅₀ (FIG. 19C).

Therefore, zongertinib inhibits the growth of HER2-dependent human NSCLC cells resistant to trastuzumab deruxtecan.

The data presented in this Example raise the exciting possibility that zongertinib may be effective in treating HER2-dependent tumors that are resistant to ADCs and, together with data from previous Examples, indicate that adding zongertinib may expand the population of patients who would benefit from HER2 targeting ADCs.

### Example 6)—A Phase Ib Dose Escalation and Phase II Dose Optimization, Randomized, Open-Label, Multicenter Trial of Oral Zongertinib in Combination with Intravenous Trastuzumab Deruxtecan (T-DXd) or in Combination with Intravenous Trastuzumab Emtansine (T-DM1) for Treatment of Patients with Advanced HER2+Metastatic Breast Cancer (mBC) and Metastatic Gastric, Gastroesophageal Junction, or Esophageal Adenocarcinoma (mGEAC)

In the present Example, a clinical trial is performed to test the combination of zongertinib with T-DXd or T-DM1 in patients with advanced HER2+metastatic breast cancer (mBC) or metastatic gastric, gastroesophageal junction, or esophageal adenocarcinoma (mGEAC).

### 6.1 Materials & Methods

The trial is designed as multicenter, randomized, open-label dose escalation (Phase Ib) and dose optimization (Phase II).

### 6.1.1 Objectives

The primary objectives of dose escalation are:
To characterize the safety, tolerability and the dose-toxicity curve of zongertinib in combination with T-DXd or T-DM1 in patients with HER2+mBC or mGEAC by assessing escalating dose levels with overdose control to achieve the primary objective of determining the maximum tolerated doses (MTDs) and/or doses for further development;
To evaluate the number of patients with dose-limiting toxicities (DLTs) within the MTD evaluation period per dose level. The MTD evaluation period is defined as the first 21 days of the first treatment cycle. The MTD is determined by the dose escalation committee (DEC) based on the totality of data. It may be chosen as the highest dose with less than 25% risk of the true DLT rate being equal to or above 33% during the MTD evaluation period based on the Bayesian Logistic Regression Model (BLRM) with overdose control (escalation with overdose control [EWOC]) for the trial;

The primary characterization of the primary objective is based on the initial dose administered to the patient during the MTD evaluation period. The strategy for handling intercurrent events is a combined composite and principal stratum approach where some intercurrent events are considered as outcome and some define the population consisting of patients who are able to adhere to the assigned treatment regimen and trial schedule.

The primary objectives of dose optimization are:
to assess the anti-tumor activity of zongertinib in combination with T-DXd or T-DM1 to assist in the selection of one out of two suitable doses per cohort for further clinical development;
the proportion of patients with objective response (OR) by RECIST version 1.1 as assessed by investigator review in the intent-to-treat population;
the summary measure of OR will include all treated patients regardless of breaks from trial treatment but will exclude the effects of any subsequent anti-cancer therapy started before progression.

Secondary objectives include:
To characterize the pharmacokinetic properties of zongertinib and T-DXd or T-DM1 when given in combination (all trial parts);
To further evaluate preliminary efficacy, safety, and the risk-benefit profile of zongertinib in combination with T-DXd or T-DM1 (all trial parts);
To evaluate patient reported outcomes (PROs) (dose optimization).

### 6.1.2 Endpoints

The primary endpoint of dose escalation is the occurrence of DLTs in the MTD evaluation period. The MTD evaluation period is defined as the first 21 days of the first treatment cycle. The primary endpoint of dose optimization is the objective response (OR) defined as the best overall response of confirmed complete response (CR) or confirmed partial response (PR) according to RECIST 1.1 from the date of treatment start until the earliest date of disease progression, death, or last evaluable tumor assessment before start of subsequent anti-cancer therapy, or treatment discontinuation as assessed by investigator review. The summary measure of OR includes all treated patients regardless of breaks from trial treatment but excludes the effects of any subsequent anti-cancer therapy started before progression. The secondary endpoints of dose escalation are:
OR, as described above;
Occurrence of DLTs during the entire treatment period;
Intensive PK sampling. In particular, the following PK parameters of zongertinib and T-DXd or T-DM1 when given in combination are evaluated:
   Cₘₐₓ: maximum measured concentration;
   AUC₀₋ₜ₂: area under the concentration-time curve.

The secondary endpoints of dose optimization are:
Progression-free survival (PFS), defined as the time from treatment start until the earliest date of tumor progression according RECIST 1.1 based on investigator review or death from any cause, whichever occurs first;
Disease control (DC) defined as best overall response of CR or PR or stable disease (SD) where best overall response is defined according to RECIST 1.1 from first treatment administration until the earliest of disease progression, death, or last evaluable tumor assessment before start of subsequent anti-cancer therapy, or treatment discontinuation, as assessed by investigator review;
Occurrence of treatment-emergent adverse events (TEAEs) leading to zongertinib dose reduction during the on-treatment period;
Sparse PK sampling. In particular, the following PK parameters of zongertinib and T-DXd or T-DM1 when given in combination are evaluated:
   Cₘₐₓ: maximum measured concentration;
   AUC₀₋ₜ₂: area under the concentration-time curve;
PROs: PRO according to the according to the Common Terminology Criteria for Adverse Events (CTCAE), e.g. Mouth/throat sores, Taste changes, Decreased appetite, Nausea, Vomiting, Constipation, Diarrhoea, Shortness of breath, Cough, Rash, Skin dryness, Hair loss, Itching, Numbness & Tingling, Fatigue, Nosebleed, Headache; EORTC IL46 (1 item); EORTC IL19 (5 items, physical functioning scale of EORTC QLQ-C30). The time frame is from first administration until an individual patient's end of treatment (EOT).

### 6.1.3 Design and Trial Population

Approximately 240 patients with histologically or cytologically confirmed advanced, unresectable HER2+overexpressing or amplified mBC or mGEAC are included in the study. Approximately 60 patients participate in dose escalation, approximately 180 in dose optimization. Patients are divided into cohorts as shown in Table 11.

TABLE 11
Schematic of the clinical trial

| | | |
|---|---|---|
| Cohort | Indication | Therapy |

Dose Escalation (Phase Ib)

| | | |
|---|---|---|
| A | mBC | Zongertinib + T-DM1 |
| B | mBC | Zongertinib + T-DXd |
| C | mGEAC | Zongertinib + T-DXd |

Dose Optimization (Phase II)

| | | |
|---|---|---|
| D | mBC | Zongertinib + T-DM1 |
| E | mBC | Zongertinib + T-DXd |
| F | mGEAC | Zongertinib + T-DXd |

In Phase Ib, escalating dose levels (DLs) of zongertinib starting at 60 mg and up to a maximum of 240 mg are investigated when co-administered with a fixed dose of T-DM1 for mBC (Cohort A), or a fixed dose of T-DXd for mBC (Cohort B) and mGEAC (Cohort C). Authorised doses of T-DXd and T-DM1 are used. Use of the reduced dose may be decided based on safety data. All cohorts are started in parallel but evaluated separately. 3 doses are planned for Cohorts A to C, with approximately 20 patients per cohort and at least 3 patients per dose. The DEC clears a dose prior to escalation and decides cohort size. It is possible that more or less doses are investigated according to DEC decisions.

In Phase II, two dose levels to be tested per indication and treatment regimen for dose optimization (Cohorts D-F) are chosen based on the data from the dose escalation part (Cohorts A to C). The lower dose will be referred to as DL1; the higher dose will be referred to as DL2. Up to 60 patients per cohort are randomized 1:1 into two DLs (n=30 per dose, per cohort). The sample size is chosen to allow for sufficient assessment of activity, safety, and tolerability for each dose.

Prior to the study, patients were previously treated with the standard of care first line and/or second line.

### 6.1.4 Inclusion and Exclusion Criteria

Main inclusion criteria are:
Patients ≥18 years of age or over the legal age of consent in countries where that is greater than 18 years at the time of signature of the informed consent form (ICF);
HER2+ (defined as immunohistochemistry [IHC]3+ or IHC 2+ and evidence of HER2 amplification by in situ hybridization [ISH]) mBC or mGEAC;
For dose optimization (Phase II): Patient must provide tumor tissue from locations not radiated prior to biopsy, if possible, collected through archival tissue;
Documented investigator assessed progression after HER2 directed treatment for advanced, metastatic disease;
Presence of at least one measurable lesion according to RECIST 1;1.
Eastern Cooperative Oncology Group (ECOG) score of 0 or 1;
Adequate organ function based on laboratory values.

Main exclusion criteria are:
Previous treatment with:
   Any small molecule HER2 inhibitor in the advanced or metastatic setting;
   T-DXd (except in Cohorts A and D);
   T-DM1 in Cohort D;
   Any experimental agent within 21 days or five half-lives of study treatment, whichever is shorter;
   Trastuzumab, pertuzumab, or all other antibody-based therapy within 3 weeks of starting study treatment; or
   Chemotherapy within 2 weeks of starting study treatment;
Presence of uncontrolled or symptomatic brain metastases, or leptomeningeal disease;
Mean resting corrected QT interval (QTcF)>470 msec.;
Any factors that increase the risk of QTc prolongation or risk of arrhythmic events such as heart failure, hypokalaemia, congenital long QT syndrome, family history of long QT syndrome or unexplained sudden death under 40 years-of-age, or any concomitant medication known to prolong the QT interval;
Ejection fraction <50% or the lower limit of normal of the institutional standard;
History of (non-infectious) interstitial lung disease (ILD)/pneumonitis that required steroids, current ILD/pneumonitis, or where suspected ILD/pneumonitis cannot be ruled out by imaging at screening.

### 6.1.5 Treatments

Zongertinib is administered orally from 60 mg QD up to 240 mg QD. T-DM1 is administered as an intravenous infusion of 3.6 mg/kg to mBC patients once every 3 weeks (21-day cycle). T-DXd is administered as an intravenous infusion of 5.4 mg/kg to mBC patients or 6.4 mg/kg to mGAEC patients once every 3 weeks (21-day cycle).

T-DM1 and T-DXd are administered according to the approved product label.

All patients are treated in cycles of 3 weeks (21 days). Patients may continue treatment until documented disease progression according to RECIST 1.1, undue toxicity, or other criteria for stopping treatment as defined in the clinical trial protocol (CTP) are met.

### 6.1.6 Statistical Methods

The analyses are descriptive and exploratory. No formal statistical test are performed. All analyses are performed separately for each cohort. For each cohort, dose escalation is guided by a Bayesian logistic regression model (BLRM) with overdose control that is fitted to binary toxicity outcomes. The estimates of parameters are updated as data are accumulated using the BLRM. At the end of dose escalation, the toxicity probability at each dose level is calculated to determine an estimate of the MTD for each cohort.

### Example 7)—Combination of Zongertinib with Trastuzumab in Xenografts Derived from OE19 (Esophagus Carcinoma Cell Line with HER2 Overexpression)

In the present Example, the anti-tumor activity of zongertinib in combination with trastuzumab is evaluated in a subcutaneous xenograft mouse model derived from the HER2-overexpressing human esophagus cancer cell line OE19 in NMRI-Foxn1^{nu} mice.

### 7.1 Materials and Methods

### 7.1.1 Study Design

The study is performed in human esophagus cancer cell line OE19 grown as subcutaneous xenografts in NMRI-Foxn1^{nu} mice. The study is designed as shown in Table 12. Day 1 was the first day, day 14 the last day of treatment and final day of analysis.

TABLE 12
Treatment groups for studying the combination of zongertinib
with trastuzumab in xenografts derived from OE19

| No. of mice | Treatment | Dose [mg/kg] | Schedule | Route |
|---|---|---|---|---|
| 8 | Control | — | qdx14/q7dx2 | p.o./i.p. |
| 8 | Zongertinib | 20 mg/kg | qdx14 | p.o. |
| 8 | Trastuzumab | 20 mg/kg | q7dx2 | i.p. |
| 8 | Zongertinib + | 20 mg/kg + | qdx14 + | p.o. + |
| | Trastuzumab | 20 mg/kg | q7dx2 | i.p. |

### 7.1.2 Cells

OE19 is obtained and cultured as described in Example 3 (see paragraph 3.1.2).

### 7.1.3 Mice

Mice are treated as described in Example 1 (see paragraph 1.1.3).

### 7.1.4 Establishment of Tumors and Randomization

Tumors are established and randomized as described in Example 1 (see paragraph 1.1.4), except that OE19 cells are used and mice are randomly distributed between the treatment and the vehicle control group (14 days after cell injection) when tumors are well established and have reached volumes of 72 to 228 mm³.

### 7.1.5 Administration of Test Compounds

Zongertinib is administered as described in Example 1 (see paragraph 1.1.5). Trastuzumab can be purchased from Roche Pharma AG, is diluted with saline (0.9%) and administered intraperitoneally with a volume of 10 ml/kg. Mice in the control group are treated with vehicle, i.e.:
(iii) 0.5% Natrosol, administered once daily for the duration of the treatment intragastrally by gavage needle with a volume of 10 ml/kg body weight; and
(iv) saline (0.9%), administered once weekly intraperitoneally with a volume of 10 ml/kg.

### 7.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 640 mm³.

### 7.1.7 Statistical Analysis

The statistical evaluation of the tumor volume and the body weight is conducted on day 14 (last day of the study). Apart from this, statistical analysis was performed as described in Example 1 (see paragraph 1.1.7), with no evaluation of tumor growth after stop of treatment.

### 7.2 Results

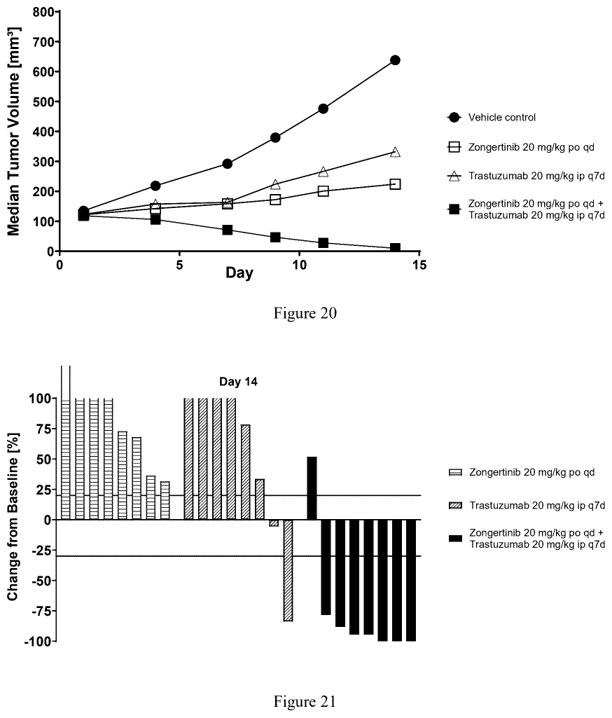

Results are summarised in Table 13 and in FIGS. 20 and 21.

TABLE 13
TGI at day 14 for each treatment group for studying the combination
of zongertinib with trastuzumab in xenografts derived from OE19

| | | | p value | p value | Regres- |
|---|---|---|---|---|---|
| | | TGI | vs. | vs. | sions |
| Compound | Dose, schedule | [%] | control | * | >30% |
| Control | — qdx14/q7dx2 | — | * | | — |
| Zongertinib | 20 mg/kg, qdx14 | 80 | 0.0019 | 0.0019 | 0/8 |
| Trastuzumab | 20 mg/kg, q7dx2 | 58 | 0.0027 | 0.0019 | 1/8 |
| Zongertinib + | 20 mg/kg, qdx14 + | 122 | 0.0007 | * | 7/8 |
| Trastuzumab | 20 mg/kg, q7dx2 | | | | |

“*” indicates reference group for comparison
bold adjusted value <0.05

All treatments were well tolerated and showed no statistically different body weight gain, compared to the controls (data not shown).

The combination showed significantly higher efficacy on tumor growth compared to both monotherapies (Table 13). Both monotherapies delayed the growth of OE19 tumors but did not shrink them, whereas the combination induced persistent tumor shrinkage (FIG. 20).

In the combination group, on day 14, 7 out of 8 tumors regressed by more than 30% (partial response), including 3 complete responses. By contrast, in the zongertinib monotherapy group there was no partial response and even no stable disease, while in the trastuzumab monotherapy group only 1 out of 8 tumors gave a partial response, 1 a stable disease and the remaining ones progressed (Table 13 and FIG. 21).

### Example 8)—Combination of Zongertinib with T-DM1 in Xenografts Derived from SUM190PT (Breast Cancer Cell Line with HER2 Overexpression)

In the present Example, the anti-tumor activity of zongertinib in combination with T-DM1 is evaluated in a subcutaneous xenograft mouse model derived from the HER2-overexpressing human breast cancer cell line SUM190PT in NMRI-Foxn1^{nu} mice.

### 8.1 Materials and Methods

### 8.1.1 Study Design

The study is performed in human breast cancer cell line SUM190PT grown as subcutaneous xenografts in NMRI-Foxn1^{nu} mice. The study is designed as shown in Table 14. Day 1 was the first day, day 21 the last day of treatment and final day of analysis.

TABLE 14
Treatment groups for studying the combination of zongertinib
with T-DM1 in xenografts derived from SUM190PT

| No. of mice | Treatment | Dose [mg/kg] | Schedule | Route |
|---|---|---|---|---|
| 8 | Control | — | qdx21/q21dx1 | p.o./i.v. |
| 8 | Zongertinib | 20 mg/kg | qdx21 | p.o. |
| 8 | T-DM1 | 3 mg/kg | q21dx1 | i.v. |
| 8 | Zongertinib + | 20 mg/kg + | qdx21 + | p.o. + |
| | T-DM1 | 3 mg/kg | q21dx1 | i.v. |

### 8.1.2 Cells

SUM190PT is a HER2-overexpressing human breast cancer cell line that can be obtained from Asterand Bioscience (BioIVT). Cells are cultured in T75, T175 and 5-Layer tissue culture flasks at 37° C. and 5% CO₂. The medium used is ACL-4 (Thermo Fisher Scientific #041-95574M Custom Batch) supplemented with 0.5% bovine serum albumin (JHR 85-041-025), 0.02 mg/ml insulin (Sigma I5500), 0.01 mg/ml transferrin (Sigma T-2252), 25 nM sodium selenite (Sigma S9133), 50 nM Hydrocortisone (Sigma H-4001), 1 ng/ml Epidermal Growth Factor (unfiltered, Sigma E4127), 0.01 mM ethanolamine (Sigma E-0135), 0.01 mM phosphorylethanolamine (Medchem HY-N5034), 100 pM triiodothyronine (Sigma T5516), 0.5 mM sodium pyruvate (Sigma S8636) and 2 mM L-glutamine (Gibco 11539876). Cultures are split once weekly with a ratio of 1:2.

### 8.1.3 Mice

Mice are treated as described in Example 1 (see paragraph 1.1.3).

### 8.1.4 Establishment of Tumors and Randomization

To establish subcutaneous tumors, SUM190PT cells are harvested by centrifugation, washed and resuspended in PBS+2.5% FCS+50% Matrigel (Corning #2165001) at 5×10⁷ cells/ml. 100 μl cell suspension containing 5×10⁶ cells is injected subcutaneously into the right flank of the mice (1 site per mouse). Mice are randomly distributed between the treatment and the vehicle control group (13 days after cell injection) when tumors are well established and have reached volumes of 100 to 180 mm³.

### 8.1.5 Administration of Test Compounds

Zongertinib, T-DM1 and vehicle are administered as described in Example 1 (see paragraph 1.1.5).

### 8.1.6 Monitoring Tumor Growth and Side Effects

Tumor volumes and side effects are monitored as described in Example 1 (see paragraph 1.1.6), except that control animals were analysed when the median tumor volume of the group reaches a size of approximately 700 mm³.

### 8.1.7 Statistical Analysis

The statistical evaluation of the tumor volume and the body weight is conducted on day 21 (last day of the study). Apart from this, statistical analysis was performed as described in Example 1 (see paragraph 1.1.7), with no evaluation of tumor growth after stop of treatment.

### 8.2 Results

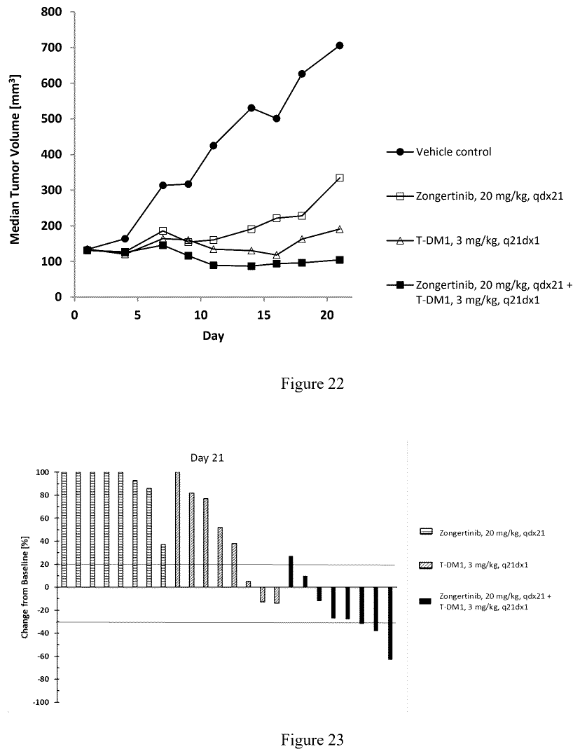

Results are summarised in Table 15 and in FIGS. 22 and 23.

TABLE 15
TGI at day 21 for each treatment group for studying the combination
of zongertinib with T-DM1 in xenografts derived from SUM190PT

| | | | p value | p value | Regres- |
|---|---|---|---|---|---|
| | | TGI | vs. | vs. | sions |
| Compound | Dose, schedule | [%] | control | * | >30% |
| Control | qdx21/q21dx1 | | * | | — |
| Zongertinib | 20 mg/kg, qdx21 | 64 | 0.0004 | 0.0002 | 0/8 |
| T-DM1 | 3 mg/kg, q21dx1 | 90 | 0.0004 | 0.0141 | 0/8 |
| Zongertinib + | 20 mg/kg, qdx21 + | 105 | 0.0004 | * | 3/8 |
| T-DM1 | 3 mg/kg, q21dx1 | | | | |

“*” indicates reference group for comparison
bold adjusted value <0.05

All treatments were well tolerated and showed no statistically different body weight gain, compared to the controls (data not shown).

The combination showed significantly higher efficacy on tumor growth compared to both monotherapies (Table 15). Both monotherapies delayed tumor growth, but median tumor volumes increased in the course of the experiment. By contrast, combining T-DM1 with zongertinib stopped tumor growth: the median tumor volume at the end of the experiment was approximately the same as at the start of the experiment (FIG. 22).

In the combination group, on day 21, 3 out of 8 tumors went into regression of more than 30% (partial response) and 4 out of 8 tumors remained stable (stable disease). By contrast, in the monotherapy groups, there were no partial responses. In the zongertinib monotherapy group, there was even no stable disease, while in the T-DM1 there were 3 (Table 15 and FIG. 23).

### Example 9)—Combination of Zongertinib with Capecitabine and Trastuzumab in Xenografts Derived from SUM190PT (Breast Cancer Cell Line with HER2 Overexpression)

In the present Example, the anti-tumor activity of zongertinib in combination with capecitabine and trastuzumab is evaluated in a subcutaneous xenograft mouse model derived from the HER2-overexpressing human breast cancer cell line SUM190PT in NMRI-Foxn1^{nu} mice.

### 9.1 Materials and Methods

### 9.1.1 Study Design

The study is performed in human breast cancer cell line SUM190PT grown as subcutaneous xenografts in NMRI-Foxn1^{nu} mice. The study is designed as shown in Table 16. Day 1 was the first day, day 21 the last day of treatment and final day of analysis.

TABLE 16
Treatment groups for studying the combination of zongertinib with
capecitabine and trastuzumab in xenografts derived from SUM190PT

| No. of mice | Treatment | Dose [mg/kg] | Schedule | Route |
|---|---|---|---|---|
| 8 | Control | — | qdx21/q7dx3 | p.o./i.p. |
| 8 | Zongertinib | 10 mg/kg | qdx21 | p.o. |
| 8 | Capecitabine | 400 mg/kg | qdx21 | p.o. |
| 8 | Trastuzumab | 20 mg/kg | q7dx3 | i.p. |
| 8 | Zongertinib + | 10 mg/kg + | qdx21 + | p.o. + |
| | Capecitabine + | 400 mg/kg + | qdx21 + | p.o. + |
| | Trastuzumab | 20 mg/kg | q7dx3 | i.p. |

### 9.1.2 Cells

Cells are obtained and cultured as described in Example 8 (see paragraph 8.1.2).

### 9.1.3 Mice

Mice are treated as described in Example 1 (see paragraph 1.1.3).

### 9.1.4 Establishment of Tumors and Randomization

Tumors are established and randomized as described in Example 8 (see paragraph 8.1.4), except that mice are randomly distributed between the treatment and the vehicle control group (19 days after cell injection) when tumors are well established and have reached volumes of 127 to 292 mm³.

### 9.1.5 Administration of Test Compounds

Zongertinib is administered as described in Example 1 (see paragraph 1.1.5). Trastuzumab and vehicle are administered as described in Example 7 (see paragraph 7.1.5). Capecitabine can be purchased from Roche Pharma AG. Capecitabine is suspended with 0.5% Natrosol and administered intragastrally with a volume of 10 ml/kg.

### 9.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 540 mm³.

### 9.1.7 Statistical Analysis

The statistical evaluation of the tumor volume and the body weight is conducted on day 21 (last day of the study). Apart from this, statistical analysis was performed as described in Example 1 (see paragraph 1.1.7), with no evaluation of tumor growth after stop of treatment.

### 9.2 Results

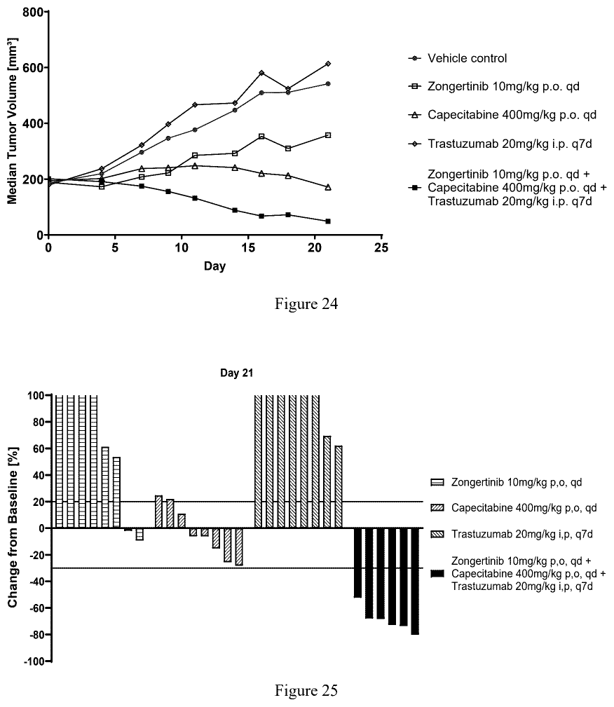

Results are summarised in Table 17 and in FIGS. 24 and 25.

TABLE 17
TGI at day 21 for each treatment group for studying the combination of zongertinib
with capecitabine and trastuzumab in xenografts derived from SUM190PT

| | | TGI | p value vs. | p value | |
|---|---|---|---|---|---|
| Compound | Dose, schedule | [%] | control | vs. * | Regressions >30% |
| Control | qdx21/q7dx3 | — | * | | — |
| Zongertinib | 10 mg/kg, qdx21 | 53 | 0.1000 | 0.0010 | 0/8 |
| Capecitabine | 400 mg/kg, qdx21 | 106 | 0.0016 | 0.0010 | 0/8 |
| Trastuzumab | 20 mg/kg, q7dx3 | −22 | 0.4000 | 0.0010 | 0/8 |
| Zongertinib + | 10 mg/kg, qdx21 + | 143 | 0.0013 | * | 6/6 |
| Capecitabine + | 400 mg/kg, qdx21 | | | | |
| Trastuzumab | 20 mg/kg, q7dx3 | | | | |

“*” indicates reference group for comparison
bold adjusted value <0.05

2 out of 8 mice treated with the triple combination lost some weight and had to be euthanized. In all other mice all treatments were well tolerated. Treatment with trastuzumab showed a significant weight gain compared to controls (data not shown).

The combination showed significantly higher efficacy on tumor growth compared to all monotherapies (Table 17). Specifically, trastuzumab alone did not even slow down tumor growth, zongertinib and capecitabine monotherapies delayed tumor growth but did not shrink tumors, while the triple combination induced persistent tumor shrinkage (FIG. 24). The triple combination was also significantly more efficacious than the dual combination of zongertinib 10 mg/kg qd+trastuzumab 20 mg/kg q7d (data not shown).

In the combination group, on day 21, 6 out of 6 tumors regressed by more than 30% (partial response). By contrast, in the monotherapy groups, there were no partial responses (Table 17 and FIG. 25) and in the dual zongertinib 10 mg/kg qd+trastuzumab 20 mg/kg q7d group there was 1 out of 8 (data not shown).

### Example 10)—Combination of Zongertinib with T-DXd and Pertuzumab in Xenografts Derived from BT-474 (Breast Cancer Cell Line with HER2 Overexpression)

In the present Example, the anti-tumor activity of zongertinib in combination with T-DXd and pertuzumab is evaluated in a subcutaneous xenograft mouse model derived from the HER2-overexpressing human breast cancer cell line BT-474 in NMRI-Foxn1^{nu} mice.

### 10.1 Materials and Methods

### 10.1.1 Study Design

The study is performed in human breast cancer cell line BT-474 grown as orthotopic xenografts in NMRI-Foxn1^{nu} mice. The study is designed as shown in Table 18. Day 1 was the first day, day 42 the last day of treatment and final day of analysis.

TABLE 18
Treatment groups for studying the combination of zongertinib
with T-DXd and pertuzumab in xenografts derived from BT-474

| No. of | | | | |
|---|---|---|---|---|
| mice | Treatment | Dose [mg/kg] | Schedule | Route |
| 8 | Control | — | qdx42/q21dx2/ | p.o./i.v./ |
| | | | q7dx6 | i.p. |
| 8 | Zongertinib | 10 mg/kg | qdx42 | p.o. |
| 8 | T-DXd | 1 mg/kg | q21dx2 | i.v. |
| 8 | Zongertinib + | 10 mg/kg + | qdx42 | p.o. |
| | T-DXd | 1 mg/kg | q21dx2 | i.v. |
| 8 | Zongertinib + | 10 mg/kg + | qdx42 | p.o. + |
| | T-DXd + | 1 mg/kg + | q21dx2 | i.v. + |
| | Pertuzumab | 20 mg/kg | q7dx6 | i.p. |

### 10.1.2 Cells

BT-474 is a HER2-overexpressing human breast cancer cell line that can be obtained from ATCC (lot #61136823). Cells are cultured in T75, T175 and 5-Layer tissue culture flasks at 37° C. and 5% CO₂. The medium used is Hybri-Care (ATCC, 46-X) supplemented with 1.5 g/L sodium bicarbonate (GIBCO, Ref.: 25080-060) and 10% FCS (Cytiva HyClone Characterized FBS, SH30084.03n). Cultures are split twice weekly with a ratio of 1:2-1:3.

### 10.1.3 Mice

Mice are treated as described in Example 1 (see paragraph 1.1.3).

### 10.1.4 Establishment of Tumors and Randomization

To establish subcutaneous tumors, BT-474 cells are harvested by centrifugation, washed and resuspended in PBS+5% FCS at 5×10⁷ cells/ml. 50 μl cell suspension containing 5×10⁶ cells is injected orthotopically into the right mammary fat pad of isoflurane anesthetized mice (1 site per mouse). Mice are randomly distributed between the treatment and the vehicle control group (20 days after cell injection) when tumors are well established and have reached volumes of 106 to 209 mm³.

### 10.1.5 Administration of Test Compounds

Zongertinib and T-DXd are administered as described in Example 1 (see paragraph 1.1.5). Pertuzumab can be purchased from Roche Pharma AG. Pertuzumab is diluted with saline (0.9%) and administered intraperitoneally with a volume of 10 ml/kg.

Mice in the control group are treated with vehicle, i.e.:
(i) 0.5% Natrosol, administered once daily for the duration of the treatment intragastrally by gavage needle with a volume of 10 ml/kg body weight; and
(ii) saline (0.9%), administered once every 3 weeks intravenously into the tail vein with a volume of 5 ml/kg; and
(iii) saline (0.9%), administered once every week intraperitoneally with a volume of 10 ml/kg.

### 10.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 470 mm³.

### 10.1.7 Statistical Analysis

The statistical evaluation of the tumor volume and the body weight is conducted on day 42 (last day of the study). Apart from this, statistical analysis was performed as described in Example 1 (see paragraph 1.1.7), with no evaluation of tumor growth after stop of treatment.

### 10.2 Results

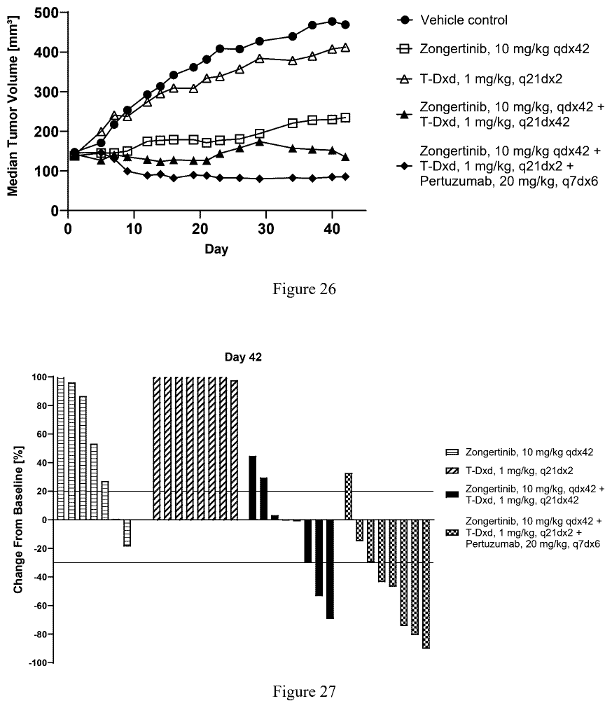

Results are summarised in Table 19 and in FIGS. 26 and 27.

TABLE 19
TGI at day 42 for each treatment group for studying the combination of
zongertinib with T-DXd and pertuzumab in xenografts derived from BT-474

| | | TGI | p value vs. | p value | |
|---|---|---|---|---|---|
| Compound | Dose, schedule | [%] | control | vs. * | Regressions >30% |
| Control | qdx42/q21dx2/q7dx6 | — | * | | — |
| Zongertinib | 10 mg/kg, qdx42 | 70 | 0.0020 | 0.0540 | 0/8 |
| T-DXd | 1 mg/kg, q21dx2 | 16 | 0.2600 | 0.0016 | 0/8 |
| Zongertinib + | 10 mg/kg, qdx42 + | 102 | 0.0003 | * | 3/8 |
| T-DXd | 1 mg/kg, q21dx2 | | | | |
| Zongertinib + | 10 mg/kg, qdx42 + | 119 | 0.0001 | 0.1200 | 5/8 |
| T-DXd + | 1 mg/kg, q21dx2 + | | | | |
| Pertuzumab | 20 mg/kg, q7dx6 | | | | |

“*” indicates reference group for comparison
bold adjusted value <0.05

All treatments were well tolerated. Triple combination showed significant less body weight gain compared to the controls (data not shown).

The dual combination of zongertinib+T-DXd showed significantly higher efficacy on tumor growth compared to both monotherapies (Table 19). Specifically, T-DXd alone had a limited effect on tumor growth. Zongertinib monotherapy delayed tumor growth, but median tumor volumes increased in the course of the experiment. By contrast, combining T-DXd with zongertinib stopped tumor growth: the median tumor volume at the end of the experiment was approximately the same as at the start of the experiment. The triple combination with pertuzumab was even more efficacious than the monotherapies and the dual therapies (FIG. 26). Pertuzumab monotherapy was not investigated because this monotherapy is not used in clinics.

In the triple combination group, on day 42, 5 out of 8 tumors regressed by more than 30% (partial response). In the dual zongertinib+T-DXd combination group, on day 42, 3 out of 8 tumors regressed by more than 30% (partial response) and another 3 out of 8 tumors remained stable. By contrast, in the monotherapy groups, there were no partial responses and there were only 2 out of 8 stable diseases in the zongertinib monotherapy group (Table 19 and FIG. 27).

### Example 11)—Combination of Zongertinib with T-DM1 in Xenografts Derived from BT-474 (Breast Cancer Cell Line with HER2 Overexpression)

In the present Example, the anti-tumor activity of zongertinib in combination with T-DM1 is evaluated in a subcutaneous xenograft mouse model derived from the HER2-overexpressing human breast cancer cell line BT-474 in NMRI-Foxn1^{nu} mice.

### 11.1 Materials and Methods

### 11.1.1 Study Design

The study is performed in human breast cancer cell line BT-474 grown as orthotopic xenografts in NMRI-Foxn1^{nu} mice. The study is designed as shown in Table 20. Day 1 was the first day, day 42 the last day of treatment and final day of analysis.

TABLE 20
Treatment groups for studying the combination of zongertinib
with T-DM1 in xenografts derived from BT-474

| No. of | | | | |
|---|---|---|---|---|
| mice | Treatment | Dose [mg/kg] | Schedule | Route |
| 8 | Control | — | qdx42/q21dx2/ | p.o./i.v./ |
| | | | q7dx6 | i.p. |
| 8 | Zongertinib | 10 mg/kg | qdx42 | p.o. |
| 8 | T-DM1 | 6 mg/kg | q21dx2 | i.v. |
| 8 | Zongertinib + | 10 mg/kg + | qdx42 | p.o. |
| | T-DM1 | 6 mg/kg | q21dx2 | i.v. |

### 11.1.2 Cells

BT-474 is obtained and cultured as described in Example 10 (see paragraph 10.1.2).

### 11.1.3 Mice

Mice are treated as described in Example 1 (see paragraph 1.1.3).

### 11.1.4 Establishment of Tumors and Randomization

Tumors are established and randomized as described in Example 10 (see paragraph 10.14), except that mice are randomly distributed between the treatment and the vehicle control group (22 days after cell injection) when tumors are well established and have reached volumes of 84 to 310 mm³.

### 11.1.5 Administration of Test Compounds

Zongertinib and T-DM1 are administered as described in Example 1 (see paragraph 1.1.5), vehicle as described in Example 10 (see paragraph 10.1.5).

### 11.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 710 mm³.

### 11.1.7 Statistical Analysis

The statistical evaluation of the tumor volume and the body weight is conducted on day 42 (last day of the study). Apart from this, statistical analysis was performed as described in Example 1 (see paragraph 1.1.7), with no evaluation of tumor growth after stop of treatment.

### 11.2 Results

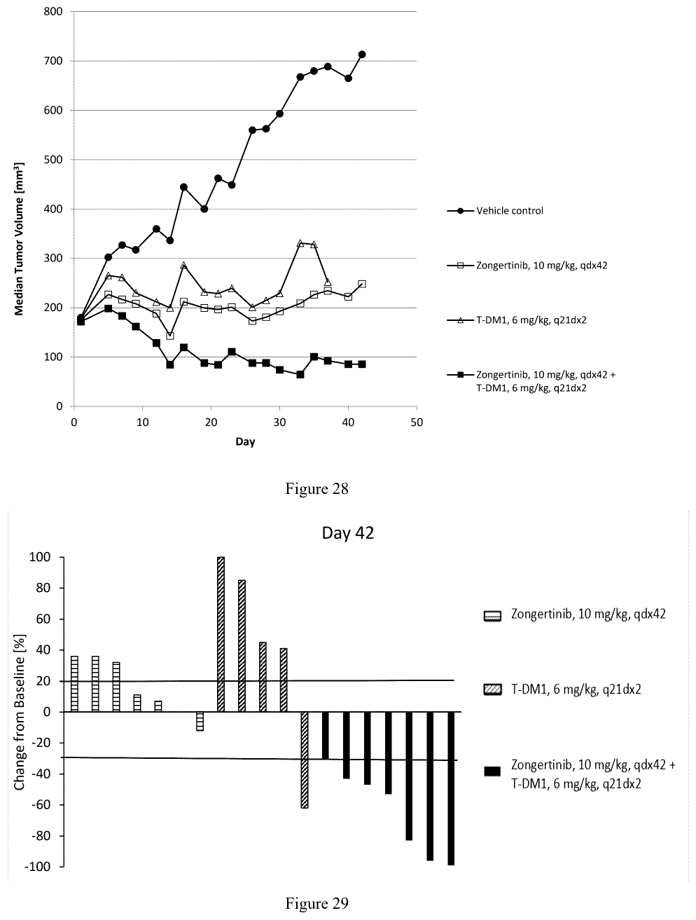

Results are summarised in Table 21 and in FIGS. 28 and 29.

TABLE 21
TGI at day 42 for each treatment group for studying the combination
of zongertinib with T-DM1 in xenografts derived from BT-474

| | | TGI | p value vs. | p value | |
|---|---|---|---|---|---|
| Compound | Dose, schedule | [%] | control | vs. * | Regressions >30% |
| Control | qdx42/q21dx2/q7dx6 | — | * | | — |
| Zongertinib | 10 mg/kg, qdx42 | 86 | 0.0006 | 0.0070 | 0/7 |
| T-DM1 | 6 mg/kg, q21dx2 | 85 | 0.0709 | 0.0256 | 1/5 |
| Zongertinib + | 10 mg/kg, qdx42 + | 116 | 0.0006 | * | 6/7 |
| T-DM1 | 6 mg/kg, q21dx2 | | | | |

“*” indicates reference group for comparison
bold adjusted value <0.05

1 out of 8 mice treated with zongertinib and 2 out of 8 treated with T-DM1 lost some weight and had to be euthanized. In all other mice all treatments were well tolerated and showed statistically higher body weight gain, compared to the controls (data not shown).

The combination showed significantly higher efficacy on tumor growth compared to both monotherapies (Table 21). Both monotherapies delayed the growth of BT-474 tumors but did not shrink them, whereas the combination induced persistent tumor shrinkage (FIG. 28).

In the combination group, on day 42, 6 out of 7 tumors regressed by more than 30% (partial response). By contrast, in the zongertinib monotherapy groups, there were no partial responses and in the T-DM1 monotherapy group, there was only 1 (out of 5). In the zongertinib monotherapy group, there was even no stable disease, while in the T-DM1 there were 3 (Table 21 and FIG. 29).

### Example 12)—Combination of Zongertinib with T-DXd in Xenografts Derived from NCI-H2170 (NSCLC Cell Line with HER2 Overexpression)

In the present Example, the anti-tumor activity of zongertinib in combination with T-DXd is evaluated in a subcutaneous xenograft mouse model derived from the HER2-overexpressing human NSCLC cell line NCI-H2170 in NMRI-Foxn1^{nu} mice.

### 12.1 Materials and Methods

### 12.1.1 Study Design

The study is performed in human NSCLC cell line NCI-H2170 grown as subcutaneous xenografts in NMRI-Foxn1^{nu} mice. The study is designed as shown in Table 22. Day 1 was the first day, day 21 the last day of treatment and final day of analysis.

TABLE 22
Treatment groups for studying the combination of zongertinib
with T-DXd in xenografts derived from NCI-H2170

| No. of mice | Treatment | Dose [mg/kg] | Schedule | Route |
|---|---|---|---|---|
| 8 | Control | — | qdx21/q21dx1 | p.o./i.v |
| 8 | Zongertinib | 20 mg/kg | qdx21 | p.o. |
| 8 | T-DXd | 3 mg/kg | q21dx1 | i.v. |
| 8 | Zongertinib + | 20 mg/kg + | qdx21 | p.o. + |
| | T-DXd | 3 mg/kg | q21dx1 | i.v. |

### 12.1.2 Cells

NCI-H2170 is a HER2-overexpressing NSCLC cell line that can be obtained from the American Type Culture Collection (ATCC #CRL-5928; original vial lot: 4419196). Cells are cultured in T175 tissue culture flasks at 37° C. and 5% CO₂. The medium used is RPMI 1640 (ATCC #30-2001) supplemented with 10% fetal bovine serum (FBS) (Gibco #26140079). Cultures are split twice weekly with a ratio of 1:3-1:9.

### 12.1.3 Mice

Mice are treated as described in Example 1 (see paragraph 1.1.3).

### 12.1.4 Establishment of Tumors and Randomization

To establish subcutaneous tumors, NCI-H2170 cells are harvested by centrifugation, washed and resuspended in PBS+5% FCS at 1×10⁷ cells/ml. 100 μl cell suspension containing 1×10⁶ cells are injected subcutaneously into the right flank of the mice (1 site per mouse). Mice are randomly distributed between the treatment and the vehicle control group (20 days after cell injection) when tumors are well established and have reached volumes of 87 to 279 mm³.

### 12.1.5 Administration of Test Compounds

Zongertinib, T-DXd and vehicle are administered as described in Example 1 (see paragraph 1.1.5).

### 12.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 1000 mm³.

### 12.1.7 Statistical Analysis

The statistical evaluation of the tumor volume and the body weight is conducted on day 21 (last day of the study). Apart from this, statistical analysis was performed as described in Example 1 (see paragraph 1.1.7), with no evaluation of tumor growth after stop of treatment.

### 12.2 Results

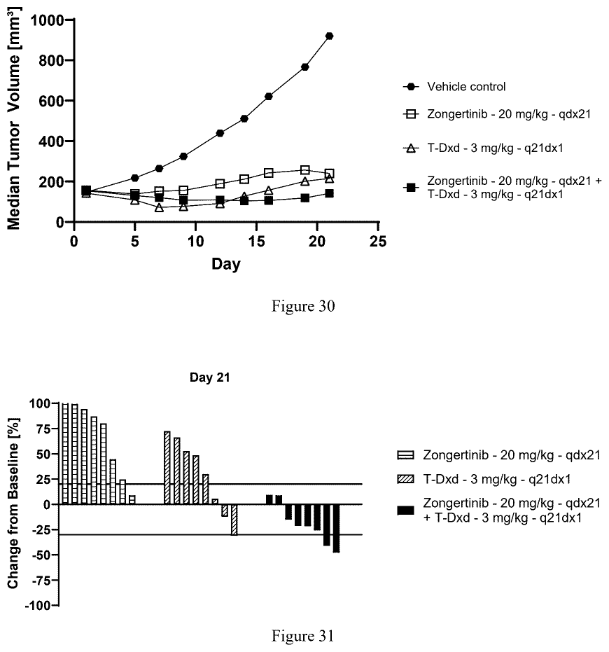

Results are summarised in Table 23 and in FIGS. 30 and 31.

TABLE 23
TGI at day 21 for each treatment group for studying the combination
of zongertinib with T-DXd in xenografts derived from NCI-H2170

| | | TGI | p value vs. | p value | |
|---|---|---|---|---|---|
| Compound | Dose, schedule | [%] | control | vs. * | Regressions >30% |
| Control | qdx21/q21dx1 | — | * | | — |
| Zongertinib | 20 mg/kg, qdx21 | 89 | 0.0002 | 0.0380 | 0/8 |
| T-DXd | 3 mg/kg, q21dx1 | 91 | 0.0002 | 0.0970 | 1/8 |
| Zongertinib + | 20 mg/kg, qdx21 + | 102 | 0.0002 | * | 2/8 |
| T-DXd | 3 mg/kg, q21dx1 | | | | |

“*” indicates reference group for comparison
bold adjusted value <0.05

All treatments were well tolerated and showed no statistically different body weight gain, compared to the controls (data not shown).

Zongertinib enhanced the efficacy of T-DXd in the HER2 amplified lung cancer model NCI-H2170 (FIG. 30).

In the combination group, on day 21, 2 out of 8 tumors went into regression of more than 30% (partial response) and the remaining 6 out of 8 tumors remained stable (stable disease). By contrast, in the zongertinib monotherapy group, there were no partial responses and only 1 stable tumor out of 8, while in the T-DXd monotherapy group, there was 1 partial response and 2 stable diseases out of 8 (Table 23 and FIG. 31).

### Example 13)—Combination of Zongertinib with T-DXd and Pertuzumab in Xenografts Derived from HCC1954 (Breast Cancer Cell Line with HER2 Overexpression)

In the present Example, the anti-tumor activity of zongertinib in combination with T-DXd and pertuzumab is evaluated in an orthotopic xenograft mouse model derived from the HER2-overexpressing human breast cancer cell line HCC1954 in NMRI-Foxn1^{nu} mice.

### 13.1 Materials and Methods

### 13.1.1 Study Design

The study is performed in human breast cancer cell line HCC1954 grown as orthotopic xenografts in NMRI-Foxn1^{nu} mice. The study is designed as shown in Table 24. Day 1 was the first day, day 21 the last day of treatment and final day of analysis.

TABLE 24
Treatment groups for studying the combination of zongertinib
with T-DXd in xenografts derived from NCI-H2170

| No. of mice | Treatment | Dose [mg/kg] | Schedule | Route |
|---|---|---|---|---|
| 8 | Control | — | qdx21/q21dx1 | p.o./i.v |
| 8 | Zongertinib | 20 mg/kg | qdx21 | p.o. |
| 8 | T-DXd | 1 mg/kg | q21dx1 | i.v. |
| 8 | Zongertinib + | 20 mg/kg + | qdx21 | p.o. + |
| | T-DXd | 1 mg/kg | q21dx1 | i.v. |
| 8 | Zongertinib + | 20 mg/kg + | qdx21 | p.o. + |
| | T-DXd + | 1 mg/kg + | q21dx1 | i.v. + |
| | Pertuzumab | 20 mg/kg | q7dx3 | i.p. |

### 13.1.2 Cells

HCC1954 is a HER2-overexpressing human breast cancer cell line that can be obtained from ATCC (lot CRL-2338). Cells are cultured in T75, T175 and 5-Layer tissue culture flasks at 37° C. and 5% CO₂. The medium used is RPMI-1640 (ATCC formulation: PAN-biotech, P04-18047) supplemented with 10% FCS (Cytiva HyClone Characterized FBS, SH30084.03n). Cultures are split twice weekly with a ratio of 1:2-1:8.

### 13.1.3 Mice

Mice are treated as described in Example 1 (see paragraph 1.1.3).

### 13.1.4 Establishment of Tumors and Randomization

To establish subcutaneous tumors, HCC1954 cells are harvested by centrifugation, washed and resuspended in PBS+5% FCS at 7×10⁷ cells/ml. 50 μl cell suspension containing 3.5×10⁶ cells is injected orthotopically into the right mammary fat pad of isoflurane anesthetized mice (1 site per mouse). Mice are randomly distributed between the treatment and the vehicle control group (14 days after cell injection) when tumors are well established and have reached volumes of 74 to 142 mm³.

### 13.1.5 Administration of Test Compounds

Zongertinib and T-DXd are administered as described in Example 1 (see paragraph 1.1.5), pertuzumab and vehicle as in Example 10 (see paragraph 10.1.5).

### 13.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 380 mm³.

### 13.1.7 Statistical Analysis

The statistical evaluation of the tumor volume and the body weight is conducted on day 21 (last day of the study). Apart from this, statistical analysis was performed as described in Example 1 (see paragraph 1.1.7), with no evaluation of tumor growth after stop of treatment.

### 13.2 Results

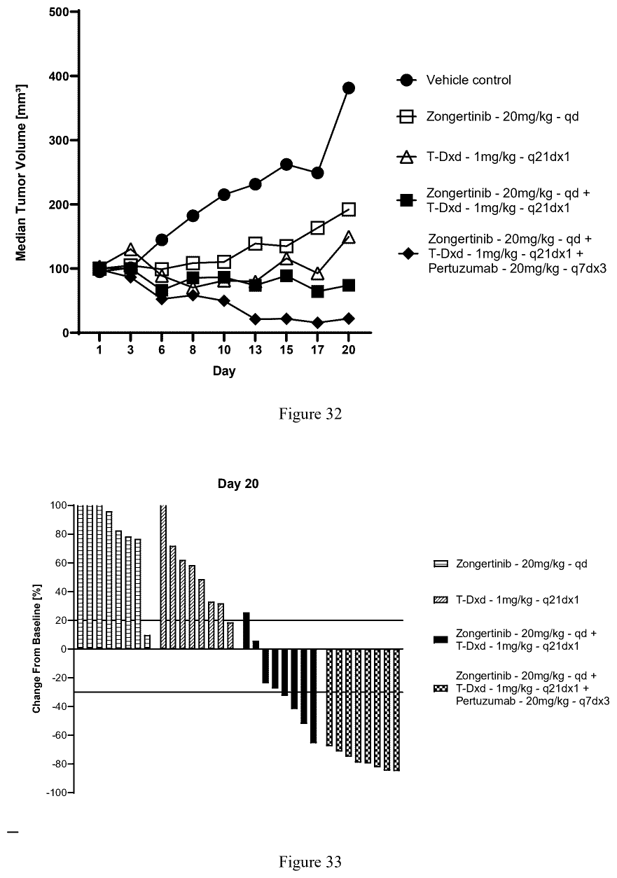

Results are summarised in Table 25 and in FIGS. 32 and 33.

TABLE 25
TGI at day 21 for each treatment group for studying the combination of zongertinib
with T-DXd and pertuzumab in xenografts derived from HCC1954

| | | TGI | p value vs. | p value | |
|---|---|---|---|---|---|
| Compound | Dose, schedule | [%] | control | vs. * | Regressions >30% |
| Control | qdx21/q21dx1/q7dx3 | — | * | | — |
| Zongertinib | 20 mg/kg, qdx21 | 68 | 0.0140 | 0.0003 | 0/8 |
| T-DXd | 1 mg/kg, q21dx1 | 84 | 0.0030 | 0.0003 | 0/8 |
| Zongertinib + | 20 mg/kg, qdx21 + | 109 | 0.0003 | * | 4/8 |
| T-DXd | 1 mg/kg, q21dx1 | | | | |
| Zongertinib + | 20 mg/kg, qdx21 + | 126 | 0.0003 | 0.0002 | 8/8 |
| T-DXd + | 1 mg/kg, q21dx1+ | | | | |
| Pertuzumab | 20 mg/kg, q7dx3 | | | | |

“*” indicates reference group for comparison
bold adjusted value <0.05

All treatments were well tolerated and only T-DXd (monotherapy) treated mice showed statistically significant less body weight gain, compared to the controls (data not shown).

The dual combination of zongertinib+T-DXd showed significantly higher efficacy on tumor growth compared to both monotherapies (Table 25). Both monotherapies delayed tumor growth, but median tumor volumes increased in the course of the experiment. By contrast, combining T-DXd with zongertinib stopped tumor growth: the median tumor volume at the end of the experiment was approximately the same as at the start of the experiment (FIG. 32). The triple combination with pertuzumab was more efficacious than the dual combination of zongertinib+T-DXd. Pertuzumab monotherapy was not investigated because this monotherapy is not used in clinics.

In the triple zongertinib+T-DXd+pertuzumab combination, all tumors regressed by more than 30%. In the dual zongertinib+T-DXd combination group, on day 20, 4 out of 8 tumors regressed by more than 30% (partial response), while there were no partial responses in either monotherapy group (Table 25 and FIG. 33).

### Example 14)—Combination of Zongertinib with Trastuzumab and Pertuzumab in Xenografts Derived from Calu-3 (NSCLC Cell Line with HER2 Overexpression)

In the present Example, the anti-tumor activity of zongertinib in combination with trastuzumab and/or pertuzumab is evaluated in a subcutaneous xenograft mouse model derived from the HER2-overexpressing human NSCLC cell line Calu-3 in CB-17/Icr-Prkdc^{scid} mice.

### 14.1 Materials and Methods

### 14.1.1 Study Design

The study is performed in human NSCLC cell line Calu-3 grown as subcutaneous xenografts in CB-17/Icr-Prkdc^{scid} mice. The study is designed as shown in Table 26. Day 1 was the first day, day 42 the last day of treatment and final day of analysis.

TABLE 26
Treatment groups for studying the combination of zongertinib with
trastuzumab and pertuzumab in xenografts derived from Calu-3

| No. of mice | Treatment | Dose [mg/kg] | Schedule | Route |
|---|---|---|---|---|
| 10 | Control | — | qdx42/q7dx6 | p.o./i.p. |
| 10 | Zongertinib | 20 mg/kg | qdx42 | p.o. |
| 10 | Trastuzumab | 20 mg/kg | q7dx6 | i.p. |
| 10 | Pertuzumab | 20 mg/kg | q7dx6 | i.p. |
| 10 | Zongertinib + | 20 mg/kg + | qdx42 | p.o. + |
| | Trastuzumab | 20 mg/kg | q7dx6 | i.p. |
| 10 | Zongertinib + | 20 mg/kg + | qdx42 | p.o. + |
| | Pertuzumab | 20 mg/kg | q7dx6 | i.p. |
| 10 | Zongertinib + | 20 mg/kg + | qdx42 | p.o. + |
| | Trastuzumab + | 20 mg/kg + | q7dx6 | i.p. + |
| | Pertuzumab | 20 mg/kg | q7dx6 | i.p. |

### 14.1.2 Cells

Calu-3 is a HER2-overexpressing human NSCLC cell line that can be obtained from ATCC (lot 614492062). Cells are cultured in T75, T175 and 5-Layer tissue culture flasks at 37° C. and 5% CO₂. The medium used is EMEM (Lonza BE12-662F) supplemented with 10% FCS (Cytiva HyClone Characterized FBS, SH30084.03n), 2 mM Glutamax (Gibco 35050-038), 0.5 mM sodium pyruvate (Sigma S8636). Cultures are split once weekly with a ratio of 1:1.5-1:2.

### 14.1.3 Mice

Mice are treated as described in Example 1 (see paragraph 1.1.3).

### 14.1.4 Establishment of Tumors and Randomization

To establish subcutaneous tumors, Calu-3 cells are harvested by centrifugation, washed and resuspended in PBS+5% FCS at 7×10⁷ cells/ml. 50 μl cell suspension containing 3.5×10⁶ cells is injected subcutaneously into the right flank of mice (1 site per mouse). Mice are randomly distributed between the treatment and the vehicle control group (15 days after cell injection) when tumors are well established and have reached volumes of 94 to 164 mm³.

### 14.1.5 Administration of Test Compounds

Zongertinib is administered as described in Example 1 (see paragraph 1.1.5), trastuzumab as described in Example 7 (see paragraph 7.1.5) and pertuzumab as described in Example 10 (see paragraph 10.1.5). Mice in the control group are treated with vehicle, i.e.:
(i) 0.5% Natrosol, administered once daily for the duration of the treatment intragastrally by gavage needle with a volume of 10 ml/kg body weight; and
(ii) saline (0.9%), administered once every week intraperitoneally with a volume of 10 ml/kg.

### 14.1.6 Monitoring Tumor Growth and Side Effects

Tumor volumes and side effects are monitored as described in Example 1 (see paragraph 1.1.6).

### 14.1.7 Statistical Analysis

The statistical evaluation of the tumor volume and the body weight is conducted on day 21 (last day of the study). Apart from this, statistical analysis was performed as described in Example 1 (see paragraph 1.1.7), with no evaluation of tumor growth after stop of treatment.

### 14.2 Results

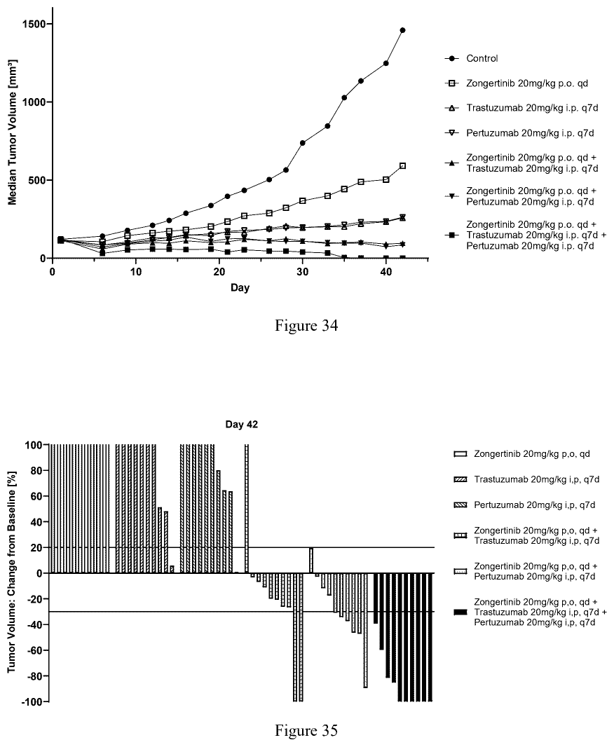

Results are summarised in Table 27 and in FIGS. 34 and 35.

TABLE 27
TGI at day 42 for each treatment group for studying the combination of zongertinib
with trastuzumab and pertuzumab in xenografts derived from Calu-3

| | | TGI | p value vs. | p value | p value | |
|---|---|---|---|---|---|---|
| Compound | Dose, schedule | [%] | control | vs. * | vs. * | Regressions >30% |
| Control | qdx42/q7dx6 | — | ** | | | — |
| Zongertinib | 20 mg/kg, qdx42 | 64 | 0.0005 | <0.0001 | | 0/10 |
| Trastuzumab | 20 mg/kg, q7dx6 | 90 | <0.0001 | 0.0001 | | 0/10 |
| Pertuzumab | 20 mg/kg, q7dx6 | 89 | <0.0001 | | | 0/10 |
| Zongertinib + | 20 mg/kg, qdx42 + | 109 | <0.0001 | | 0.0020 | 2/10 |
| Trastuzumab | 20 mg/kg, q7dx6 | | | | | |
| Zongertinib + | 20 mg/kg, qdx42 + | 101 | <0.0001 | * | 0.0003 | 6/10 |
| Pertuzumab | 20 mg/kg, q7dx6 | | | | | |
| Zongertinib + | 20 mg/kg, qdx42 + | 109 | <0.0001 | | * | 10/10 |
| Trastuzumab + | 20 mg/kg, q7dx6 | | | | | |
| Pertuzumab | 20 mg/kg, q7dx6 | | | | | |

“*” indicates reference group for comparison
bold adjusted value <0.05

All treatments were well tolerated and showed no statistically different body weight gain, compared to the controls (data not shown).

The triple combination showed significantly higher efficacy on tumor growth compared to all monotherapies (Table 27). The zongertinib+trastuzumab and zongertinib+pertuzumab dual combinations showed significantly higher efficacy on tumor growth compared to the respective monotherapies (Table 27). Specifically, all monotherapies delayed tumor growth, but median tumor volumes increased in the course of the experiment. By contrast, combining trastuzumab and/or pertuzumab with zongertinib shrank tumors (FIG. 34).

In the triple combination group all tumors regressed by more than 30% (partial response) and 6 out of 10 tumors achieved a complete response. In the dual zongertinib+trastuzumab combination group, on day 42, 2 out of 10 tumors achieved a complete response and in the dual zongertinib+pertuzumab combination group, on day 42, 6 out of 10 tumors regressed by more than 30% (partial response). By contrast, no partial response was observed in the monotherapy groups (Table 27 and FIG. 35).

### Example 15)—Combination of Zongertinib with T-DXd in Xenografts Derived from SK-OV-3 (Ovarian Cancer Cell Line with HER2 Overexpression)

In the present Example, the anti-tumor activity of zongertinib in combination with T-DXd is evaluated in a subcutaneous xenograft mouse model derived from the HER2-overexpressing human ovarian cell line SK-OV-3 in NMRI-Foxn1^{nu} mice.

### 15.1 Materials and Methods

### 15.1.1 Study Design

The study is performed in human ovarian cancer cell line SK-OV-3 grown as subcutaneous xenografts in NMRI-Foxn1^{nu} mice. The study is designed as shown in Table 28. Day 1 was the first day, day 20 the last day of treatment and final day of analysis. In the present Example, q21dx1 means that T-DXd is administered once during the 20-day experiment.

TABLE 28
Treatment groups for studying the combination of zongertinib
with T-DXd in xenografts derived from SK-OV-3

| No. of mice | Treatment | Dose [mg/kg] | Schedule | Route |
|---|---|---|---|---|
| 8 | Control | — | qdx20/q21dx1 | p.o./i.v |
| 8 | Zongertinib | 10 mg/kg | qdx20 | p.o. |
| 8 | T-DXd | 7 mg/kg | q21dx1 | i.v. |
| 8 | Zongertinib + | 10 mg/kg + | qdx20 | p.o. + |
| | T-DXd | 7 mg/kg | q21dx1 | i.v. |

### 15.1.2 Cells

SK-OV-3 is a HER2-overexpressing human ovarian cancer cell line that can be obtained from ATCC (lot HTB-77). Cells are cultured in T75, T175 and 5-Layer tissue culture flasks at 37° C. and 5% CO₂. The medium used is IMDM (PAN-Biotech, #P04-200150) supplemented with 10% FCS (Cytiva HyClone Characterized FBS, SH30084.03n). Cultures are split twice weekly with a ratio of 1:2-1:3.

### 15.1.3 Mice

Mice are treated as described in Example 1 (see paragraph 1.1.3).

### 15.1.4 Establishment of Tumors and Randomization

To establish subcutaneous tumors, SK-OV-3 cells are harvested by centrifugation, washed and resuspended in PBS+5% FCS at 5×10⁷ cells/ml. 100 μl cell suspension containing 5×10⁶ cells is injected subcutaneously into the right flank of mice (1 site per mouse). Mice are randomly distributed between the treatment and the vehicle control group (15 days after cell injection) when tumors are well established and have reached volumes of 104 to 218 mm³.

### 15.1.5 Administration of Test Compounds

Zongertinib, T-DXd and vehicle are administered as described in Example 1 (see paragraph 1.1.5).

### 15.1.6 Monitoring Tumor Growth and Side Effects

Tumor volumes and side effects are monitored as described in Example 1 (see paragraph 1.1.6), except that control animals are sacrificed when the median tumor volume of the group reaches a size of approximately 1340 mm³ (day 15).

### 15.1.7 Statistical Analysis

The statistical evaluation of the tumor volume and the body weight is conducted on day 15 (last day of the controls). Apart from this, statistical analysis was performed as described in Example 1 (see paragraph 1.1.7), with no evaluation of tumor growth after stop of treatment.

### 15.2 Results

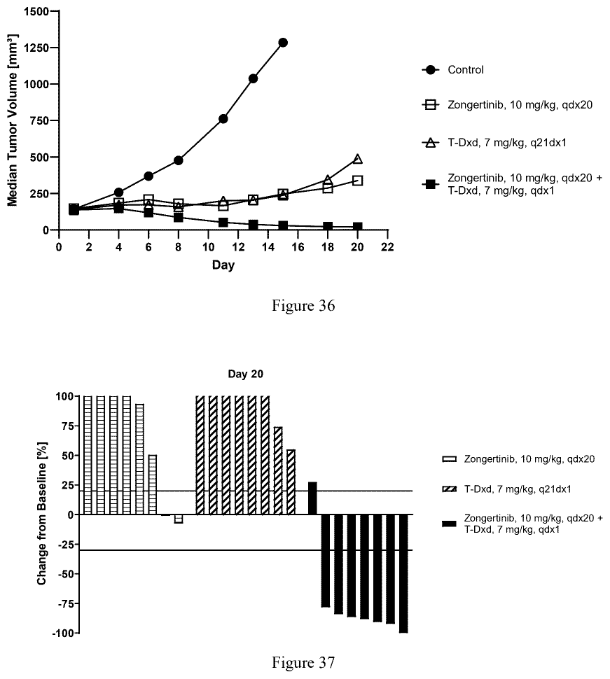

Results are summarised in Table 29 and in FIGS. 36 and 37.

TABLE 29
TGI at day 15 for each treatment group for studying the combination
of zongertinib with T-DXd in xenografts derived from SK-OV-3

| | | TGI | p value vs. | p value | |
|---|---|---|---|---|---|
| Compound | Dose, schedule | [%] | control | vs. * | Regressions >30% |
| Control | qdx20/q21dx1 | — | * | | — |
| Zongertinib | 10 mg/kg, qdx20 | 91 | 0.0005 | 0.0003 | 0/8 |
| T-DXd | 7 mg/kg, q21dx1 | 91 | 0.0005 | 0.0002 | 0/8 |
| Zongertinib + | 10 mg/kg, qdx20 + | 109 | 0.0005 | * | 7/8 |
| T-DXd | 7 mg/kg, q21dx1 | | | | |

“*” indicates reference group for comparison
bold adjusted value <0.05

All treatments were well tolerated and showed no statistically different body weight gain, compared to the controls (data not shown).

The combination showed significantly higher efficacy on tumor growth compared to both monotherapies (Table 29). Both monotherapies delayed the growth of SK-OV-3 tumors but did not shrink them, whereas the combination induced persistent tumor shrinkage (FIG. 36).

In the combination group, on day 20, 7 out of 8 tumors regressed by more than 30% (partial response), including 1 complete response. By contrast, there were no partial responses in the monotherapy groups and only 2 out of 8 stable diseases in the zongertinib monotherapy group (Table 29 and FIG. 37).

### Example 16)—a Phase Ib Dose Escalation and Phase II Dose Optimization, Randomized, Open-Label, Multicenter Trial of Oral Zongertinib Alone or in Combination with Other Agents for the Treatment of Patients with Advanced HER2+Metastatic Breast Cancer (mBC) and Metastatic Gastric, Gastroesophageal Junction, or Esophageal Adenocarcinoma (mGEAC)

In the present Example, some changes are made to the trial of Example 6, in particular to include the combination of zongertinib with trastuzumab and the combination of zongertinib with trastuzumab and capecitabine.

### 16.1 Materials & Methods

The trial is designed as multicenter, randomized, open-label dose escalation (Phase Ib) and dose optimization (Phase II).

### 16.1.1 Objectives

The primary objectives of dose escalation are:
To characterize the safety, tolerability and the dose-toxicity curve of zongertinib in combination with T-DXd, in combination with trastuzumab and capecitabine, or in combination with T-DM1 in patients with HER2+mBC by assessing escalating dose levels with overdose control to achieve the primary objective of determining the maximum tolerated doses (MTDs) and/or doses for further development. For mGEAC only the combination of zongertinib with T-DXd is investigated;
To evaluate the number of patients with dose-limiting toxicities (DLTs) within the MTD evaluation period per dose level. The MTD evaluation period is defined as the first 21 days of the first treatment cycle. The MTD is determined by the dose escalation committee (DEC) based on the totality of data. It may be chosen as the highest dose with less than 25% risk of the true DLT rate being equal to or above 33% during the MTD evaluation period based on the Bayesian Logistic Regression Model (BLRM) with overdose control (escalation with overdose control [EWOC]) for the trial;

The primary characterization of the primary objective is based on the initial dose administered to the patient during the MTD evaluation period. The strategy for handling intercurrent events is a combined composite and principal stratum approach where some intercurrent events are considered as outcome and some define the population consisting of patients who are able to adhere to the assigned treatment regimen and trial schedule.

The primary objectives of dose optimization are:
to assess the anti-tumor activity of zongertinib in the following settings to assist in the selection of optimal dose for further clinical development:
   in combination with T-DM1 in patients with mBC (Cohort D)
   in combination with T-DXd in patients with mBC (Cohort E)
   in combination with T-DXd in patients with mGEAC (Cohort F)
   in combination with capecitabine and trastuzumab in patients with mBC (Cohort H)
   as a monotherapy in patients with mBC (Cohort I, I-ext)
   in combination with trastuzumab in patients with mBC (Cohort J, J-ext);
the proportion of patients with objective response (OR) by RECIST version 1.1 as assessed by investigator review in the intent-to-treat population;
the summary measure of OR will include all treated patients regardless of breaks from trial treatment but will exclude the effects of any subsequent anti-cancer therapy started before progression.

Secondary objectives include:
To characterize the pharmacokinetic properties of zongertinib when given as monotherapy or in combination (all trial parts);
To further evaluate preliminary efficacy, safety, and the risk-benefit profile of zongertinib monotherapy and zongertinib in combination: with trastuzumab with or without capecitabine, with T-DXd, or with T-DM1 (all trial parts);
To evaluate patient reported outcomes (PROs) (dose optimization).

### 16.1.2 Endpoints

The primary endpoint of dose escalation is the occurrence of DLTs in the MTD evaluation period. The MTD evaluation period is defined as the first 21 days of the first treatment cycle. The primary endpoint of dose optimization is the objective response (OR) defined as the best overall response of confirmed complete response (CR) or confirmed partial response (PR) according to RECIST 1.1 from the date of treatment start until the earliest date of disease progression, death, or last evaluable tumor assessment before start of subsequent anti-cancer therapy, or treatment discontinuation as assessed by investigator review.

The secondary endpoints of dose escalation are:
OR, as described above;
Occurrence of DLTs during the entire treatment period;
Intensive PK sampling. In particular, the following PK parameters of zongertinib when given in combination are evaluated, if feasible:
   Cₘₐₓ: maximum measured concentration (at steady state);
   AUC_{0-4h,ss}: Area under the concentration-time curve over the time interval from 0 to 4h at steady state
   AUC_{0-tz,ss}: area under the concentration-time curve over the time interval from 0 to the last quantifiable data point at steady state.

The secondary endpoints of dose optimization are:
Progression-free survival (PFS), defined as the time from treatment start until the earliest date of tumor progression according to RECIST 1.1 based on investigator review or death from any cause, whichever occurs first;
Disease control (DC) defined as best overall response of CR or PR or stable disease (SD) where best overall response is defined according to RECIST 1.1 from first treatment administration until the earliest of disease progression, death, or last evaluable tumor assessment before start of subsequent anti-cancer therapy, or treatment discontinuation, as assessed by investigator review;
Occurrence of treatment-emergent adverse events (TEAEs) leading to zongertinib dose reduction during the on-treatment period;
Sparse PK sampling. In particular, the following PK parameters of zongertinib when given in combination are evaluated, if feasible:
   Cₘₐₓ₍ₛₛ₎: maximum measured concentration (at steady state);
   AUC_{0-tz,ss}: area under the concentration-time curve over the time interval from 0 to the last quantifiable data point at steady state.
PROs: PRO according to the Common Terminology Criteria for Adverse Events (CTCAE), e.g. Mouth/throat sores, Taste changes, Decreased appetite, Nausea, Vomiting, Constipation, Diarrhoea, Shortness of breath, Cough, Rash, Skin dryness, Hair loss, Itching, Numbness & Tingling, Fatigue, Nosebleed, Headache; EORTC IL46 (1 item, overall side effect implant); EORTC IL19 (5 items, physical functioning scale of EORTC QLQ-C30). The time frame is from first administration until an individual patient's end of treatment (EoT).

### 16.1.3 Design and Trial Population

Approximately 582 patients with histologically or cytologically confirmed unresectable, locally advanced or metastatic HER2+overexpressing or amplified BC or GEAC who were previously treated with the standard of care (depending on the cohort) can be included in the study. Approximately 80 patients participate in dose escalation, approximately 360 in dose optimization and approximately 70 patients in the extension of certain cohort and up to 72 patients on dose escalation in backfill cohorts. As soon as the DEC has declared a dose level as safe, the sponsor may decide to open the dose level for additional patients. This will be known as ‘backfilling’ of the cohort. Additional patients (up to 7 patients) will be added up to a maximum of 10 patients in total per dose level, to generate safety, tolerability, and efficacy data in a higher number of patients. Patients are divided into cohorts as shown in Table 30.

TABLE 30
Schematic of the clinical trial

| | | |
|---|---|---|
| Cohort | Indication | Therapy |

Dose Escalation (Phase Ib)

| | | |
|---|---|---|
| A | mBC | Zongertinib + T-DM1 |
| B | mBC | Zongertinib + T-DXd |
| C | mGEAC | Zongertinib + T-DXd |
| G | mBC | Zongertinib + trastuzumab + capecitabine |

Dose Optimization (Phase II)

| | | |
|---|---|---|
| D | mBC | Zongertinib + T-DM1 |
| E | mBC | Zongertinib + T-DXd |
| F | mGEAC | Zongertinib + T-DXd |
| H | mBC | Zongertinib + trastuzumab + capecitabine |
| I | mBC | Zongertinib |
| J | mBC | Zongertinib + trastuzumab |

Phase II extension

| | | |
|---|---|---|
| I-ext | mBC | Zongertinib |
| J-ext | mBC | Zongertinib + trastuzumab |

In Phase Ib, escalating dose levels (DLs) of zongertinib starting at 60 mg and up to a maximum of 360 mg are investigated when co-administered with a fixed dose of T-DM1 for mBC (Cohort A), or a fixed dose of T-DXd for mBC (Cohort B) and mGEAC (Cohort C) or a fixed dose of trastuzumab with capecitabine (Cohort G). Authorised doses of T-DXd, T-DM1, trastuzuman and capecitabine are used. Use of the reduced dose may be decided based on safety data. All cohorts are started in parallel but evaluated separately. While 4 doses are planned for Cohorts A-B-C-G, it is possible that more or fewer doses are investigated according to DEC decisions.

In Phase II, two dose levels to be tested per indication and treatment regimen for dose optimization (Cohorts D-E-F-H) are chosen based on the data from the dose escalation part (Cohorts A-B-C-G). The lower dose will be referred to as DL1; the higher dose will be referred to as DL2. For Cohorts I, I-ext, J, and J-ext the dose at DL1 will be 180 mg and DL2 will be 360 mg.

Prior to the study, patients were previously treated with the standard of care first line and/or second line.

### 16.1.4 Inclusion and Exclusion Criteria

Main inclusion criteria are:
Patients ≥18 years of age or over the legal age of consent in countries where that is greater than 18 years at the time of signature of the informed consent form (ICF);
Documented HER2+ (defined as immunohistochemistry [IHC]3+ or IHC 2+ and evidence of HER2 amplification by in situ hybridization [ISH]) mBC or mGEAC;
For dose optimization (Phase II): Patient must provide tumor tissue from locations not radiated prior to biopsy, if possible, collected through archival tissue;
Documented investigator assessed progression after HER2 directed treatment for unresectable locally advanced or metastatic disease. For cohorts Cohorts D, H, I, (I-ext), J (and J-ext)-patients must have been pretreated with T-DXd and have progressed or have been intolerant to previous T-DXd);
Presence of at least one measurable lesion according to RECIST 1.1;
Eastern Cooperative Oncology Group (ECOG) score of 0 or 1;
Adequate organ function based on laboratory values.

Main exclusion criteria are:
Previous treatment with:
   Any small molecule HER2 inhibitor in the advanced or metastatic setting in Cohorts D, E, F, and H. In Cohort D allowed in up to 15 patients in each DL;
   T-DXd in Cohorts E and F;
   T-DM1 in Cohort D and H. In Cohort H allowed in up to 15 patients in each DL;
   Capecitabine in Cohort D and H. In Cohort D allowed in up to 15 patients in each Dose level (DL);
Presence of uncontrolled and/or symptomatic brain metastases, or leptomeningeal disease;
Mean resting corrected QT interval (QTcF)>470 msec.;
Any factors that increase the risk of QTc prolongation or risk of arrhythmic events such as heart failure, hypokalaemia, congenital long QT syndrome, personal or family history of long QT syndrome or unexplained sudden death under 40 years-of-age, or any concomitant medication known to prolong the QT interval;
Ejection fraction <50% or the lower limit of normal of the institutional standard within 28 days prior to randomization;
History of (non-infectious) interstitial lung disease (ILD)/pneumonitis that required steroids, current ILD/pneumonitis, or where suspected ILD/pneumonitis cannot be ruled out by imaging at screening.

### 16.1.5 Treatments

Zongertinib is administered orally from 60 mg QD up to 360 mg QD. T-DM1 is administered as an intravenous infusion of 3.6 mg/kg to mBC patients once every 3 weeks (21-day cycle). T-DXd is administered as an intravenous infusion of 5.4 mg/kg to mBC patients or 6.4 mg/kg to mGAEC patients once every 3 weeks (21-day cycle). Capecitabine 1000 mg/m² is administered orally BID on days 1-14 of each 21 day cycle. If patients have not received trastuzumab within 4 weeks of the first day of the cycle, a loading dose of 8 mg/kg intravenously is administered, followed by once every 3 weeks (21 day cycle) 6 mg/kg.

If patients have received trastuzumab within 4 weeks of the first day of the cycle, 6 mg/kg intravenously once every 3 weeks (21 day cycle) is administered.

T-DM1, T-DXd, trastuzumab and capecitabine are administered according to the approved product label.

All patients are treated in cycles of 3 weeks (21 days). Patients may continue treatment until documented disease progression according to RECIST 1.1, undue toxicity, or other criteria for stopping treatment as defined in the clinical trial protocol (CTP) are met.

### 16.1.6 Statistical Methods

The analyses are descriptive and exploratory. No formal statistical test is performed. All analyses are performed separately for each cohort. For each cohort in the Phase Ib part, dose escalation is guided by a Bayesian logistic regression model (BLRM) with overdose control that is fitted to binary toxicity outcomes. The estimates of parameters are updated as data are accumulated using the BLRM. At the end of dose escalation, the toxicity probability at each dose level is calculated to determine an estimate of the MTD for each cohort.

1.-39. (canceled)

40. A method of treating cancer, the method comprising administering to a patient in need thereof a therapeutically effective amount of a combination comprising:
zongertinib or a pharmaceutically acceptable salt thereof, and
a combination partner,
wherein said combination partner is selected from the group consisting of an anti-HER2 antibody, an anti-HER2 antibody-drug conjugate and a combination thereof.

41. The method according to claim 40, wherein the combination further comprises an anti-cancer medicament.

42. The method according to claim 40, wherein the combination partner is an anti-HER2 antibody or an anti-HER2 antibody-drug conjugate.

43. The method according to claim 40, wherein the combination partner is an anti-HER2 antibody.

44. The method according to claim 43, wherein the anti-HER2 antibody is selected from the group consisting of trastuzumab, pertuzumab, margetuximab, zanidatamab, disitamab, anvatabart, hersintuzumab, anbenitamab and a combination thereof.

45. The method according to claim 40, wherein the combination partner is an anti-HER2 antibody-drug conjugate.

46. The method according to claim 40, wherein the anti-HER2 antibody-drug conjugate is selected from the group consisting of trastuzumab deruxtecan, trastuzumab emtansine, trastuzumab rezetecan, trastuzumab duocarmazine, zanidatamab zovodotin, disitamab vedotin, anvatabart opadotin and a combination thereof.

47. The method according to claim 41, wherein the anti-cancer medicament is selected from the group consisting of capecitabine, eribulin, docetaxel, 5-fluorouracil, folonic acid and a combination thereof.

48. The method according to claim 40, wherein zongertinib or the pharmaceutically acceptable salt thereof is administered daily.

49. The method according to claim 40, wherein zongertinib or the pharmaceutically acceptable salt thereof is administered in a daily dose of 60 mg to 360 mg.

50. The method according to claim 40, wherein the cancer is selected from the group consisting of breast cancer, esophageal cancer, gastric cancer, gastroesophageal junction cancer, lung cancer and ovarian cancer.

51. The method according to claim 40, wherein the cancer is HER2 overexpressed, HER2 amplified and/or HER2 mutant.

52. The method according to claim 40, wherein the cancer is resistant to treatment with the combination partner.

53. The method according to claim 40, wherein the combination produces a synergistic therapeutic effect as compared to the sole administration of zongertinib or the pharmaceutically acceptable salt thereof, or of the sole administration of an individual combination partner.

54. The method according to claim 41, wherein the combination produces a synergistic therapeutic effect as compared to the sole administration of:
zongertinib or the pharmaceutically acceptable salt thereof;
an individual combination partner; and/or
the anti-cancer medicament.

55. The method of claim 41 wherein the combination produces a synergistic therapeutic effect as compared to the administration of:
zongertinib or the pharmaceutically acceptable salt thereof, and an individual combination partner;
zongertinib or the pharmaceutically acceptable salt thereof, and the anti-cancer medicament; and/or
an individual combination partner and the anti-cancer medicament.

56. The method of claim 40, wherein the zongertinib or a pharmaceutically acceptable salt thereof, and the combination partner are administered concurrently.

57. A method of treating cancer, the method comprising administering to a patient in need thereof a therapeutically effective amount of zongertinib or a pharmaceutically acceptable salt thereof, wherein the cancer is resistant to treatment with an anti-HER2 antibody and/or with an anti-HER2 antibody-drug conjugate.

58. The method according to claim 57, wherein the cancer is resistant to treatment with an anti-HER2 antibody, and wherein the anti-HER2 antibody is selected from the group consisting of trastuzumab, pertuzumab, margetuximab, zanidatamab, disitamab, anvatabart, hersintuzumab, anbenitamab and a combination thereof.

59. The method according to claim 57, wherein the cancer is resistant to treatment with an anti-HER2 antibody-drug conjugate, and wherein the anti-HER2 antibody-drug conjugate is selected from the group consisting of trastuzumab deruxtecan, trastuzumab emtansine, trastuzumab rezetecan, trastuzumab duocarmazine, zanidatamab zovodotin, disitamab vedotin, anvatabart opadotin and a combination thereof.

60. The method according to claim 57, wherein zongertinib or the pharmaceutically acceptable salt thereof is administered daily.

61. The method according to claim 57, wherein zongertinib or the pharmaceutically acceptable salt thereof is administered in a daily dose of 60 mg to 360 mg.

62. The method according to claim 57, wherein the cancer is selected from the group consisting of breast cancer, esophageal cancer, gastric cancer, gastroesophageal junction cancer, lung cancer and ovarian cancer.

63. The method according to claim 57, wherein the cancer is HER2 overexpressed, HER2 amplified and/or HER2 mutant.

64. A kit comprising:
(i) a first pharmaceutical composition comprising a therapeutically effective amount of zongertinib or a pharmaceutically acceptable salt thereof, and a pharmaceutically acceptable excipient; and
(ii) a second pharmaceutical composition comprising a therapeutically effective amount of a combination partner and a pharmaceutically acceptable excipient;
wherein the combination partner is selected from the group consisting of an anti-HER2 antibody, an anti-HER2 antibody-drug conjugate, and a combination thereof; and wherein the first and second pharmaceutical compositions are packaged together in a single container.

65. The kit according to claim 64, further comprising:
(iii) a third pharmaceutical composition comprising a therapeutically effective amount of an anti-cancer medicament and a pharmaceutically acceptable excipient, wherein the third pharmaceutical composition is packaged in the single container.

66. The kit according to claim 64, wherein the combination partner is an anti-HER2 antibody or an anti-HER2 antibody-drug conjugate.

67. The kit according to claim 64, wherein the combination partner comprises an anti-HER2 antibody.

68. The kit according to claim 67, wherein the anti-HER2 antibody is selected from the group consisting of trastuzumab, pertuzumab, margetuximab, zanidatamab, disitamab, anvatabart, hersintuzumab, anbenitamab and a combination thereof.

69. The kit according to claim 64, wherein the combination partner is an anti-HER2 antibody-drug conjugate.

70. The kit according to claim 69, wherein the anti-HER2 antibody-drug conjugate is selected from the group consisting of trastuzumab deruxtecan, trastuzumab emtansine, trastuzumab rezetecan, trastuzumab duocarmazine, zanidatamab zovodotin, disitamab vedotin, anvatabart opadotin and a combination thereof.

71. The kit according to claim 65, wherein the anti-cancer medicament is selected from the group consisting of capecitabine, eribulin, docetaxel, 5-fluorouracil, folonic acid and a combination thereof.

72. The kit according to claim 64, further comprising instructions which specify a dose regimen for zongertinib or the pharmaceutically acceptable salt thereof as daily administration.

73. The kit according to claim 72, wherein the instructions specify a daily dose of zongertinib or the pharmaceutically acceptable salt thereof, of 60 mg to 360 mg.

74. The kit according to claim 64, further comprising instructions for administering the first and second pharmaceutical compositions concurrently.

75. The kit according to claim 64, further comprising instructions specifying a type of cancer to treat and wherein the type of cancer is selected from the group consisting of breast cancer, esophageal cancer, gastric cancer, gastroesophageal junction cancer, lung cancer and ovarian cancer.

76. The kit according to claim 64, further comprising instructions specifying a type of cancer to treat and wherein the type of cancer is HER2 overexpressed, HER2 amplified and/or HER2 mutant cancer.

77. The kit according to claim 64, further comprising instructions specifying a type of cancer to treat and wherein the type of cancer is a cancer that is resistant to treatment with the combination partner.

78. The kit according to claim 64, further comprising instructions specifying at least one dosage that produces a synergistic therapeutic effect in a patient as compared to the sole administration of zongertinib or the pharmaceutically acceptable salt thereof, or the sole administration of an individual combination partner.

79. The kit according to claim 65, further comprising instructions specifying at least one dosage regimen that produces a synergistic therapeutic effect in a patient as compared to the sole administration of:
zongertinib or the pharmaceutically acceptable salt thereof;
the combination partner; and/or
the anti-cancer medicament.

80. The kit according to claim 65, further comprising instructions specifying at least one dosage regimen that produces a synergistic therapeutic effect as compared to the administration of:
zongertinib or the pharmaceutically acceptable salt thereof, and an individual combination partner;
zongertinib or the pharmaceutically acceptable salt thereof, and the anti-cancer medicament; and/or
an individual combination partner and the anti-cancer medicament.
